(19) United States
(12) Patent Application Publication
(10) Pub. No.: US 2008/0114005 A1
(43) Pub. Date: May 15, 2008

(54) Fibrate Compounds Having Ppar Agonist Activity

(75) Inventors: Saibal Kumar Das, Hyderabad (IN); Sunil Kumar Singh, Hyderabad (IN); Gurram Ranga Madhavan, Hyderabad (IN); Debnath Bhuniya, Hyderabad (IN); Javed Iqbal, Hyderabad (IN); Sudhir Kumar Sharma, Hyderabad (IN); Ranjan Chakrabarti, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: Dr. Reddy's Laboratories Limited, Hyderabad, Andhra Pradesh (IN); Dr. Reddy's Laboratories, Inc., Bridgewater, NJ (US)

(21) Appl. No.: 11/574,702

(22) PCT Filed: Sep. 6, 2005
(86) PCT No.: PCT/US05/31532

§ 371 Date: Mar. 5, 2007

(30) Foreign Application Priority Data
Sep. 6, 2004 (IN) 897/CHE/2004

(51) Int. Cl. A61K 31/519; C07D 487/02

(52) U.S. Cl. 514/263.3; 544/262

(57) ABSTRACT

There are provided derivatives having PPAR agonist activity. The derivatives include compounds and/or their pharmaceutically acceptable salts; the compounds having the formula (I) wherein A has the structure (II) or (III); X is chosen from —CH₂—, —O—, —NH—, and —S—; Y is chosen from —O—, —NH—, and —S—; Z, which may be located in any position of substitution, is hydrogen or halogen; R¹ and R², which may be the same or different, are independently chosen from hydrogen and C₁-C₈ alkyl, or R¹ and R² together form a carbocyclic ring having from 4 to 6 carbon atoms; R³ is chosen from hydrogen and C₁-C₈ alkyl; R⁴, R⁵, and R⁶, which may be the same or different, are independently chosen from hydrogen and C₁-C₈ alkyl; and n is 1 to 6. Various embodiments and variants are provided. In accordance with other aspects, the invention also provides methods of producing a PPARα agonist activity in a mammal, the methods including administering to the mammal an effective amount of certain derivative(s) of the first aspect of the invention, a method of producing a PPARα agonist activity and a PPARα agonist activity in a mammal, the method including administering to the mammal an effective amount of certain derivative(s); and a pharmaceutical composition that includes the derivative(s) of the first aspect of the invention and one or more pharmaceutically-acceptable excipients. Various embodiments and variants are provided.

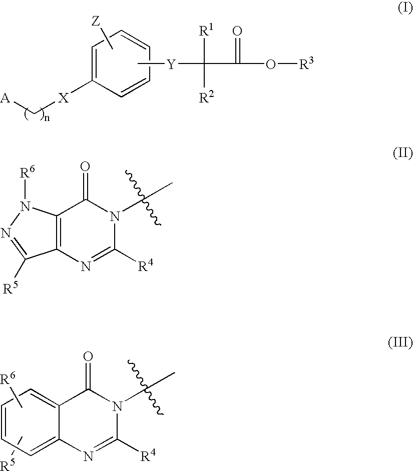

### BACKGROUND

Peroxisome Proliferator Activated Receptors (PPARs) are orphan receptors belonging to the steroid/retinoid receptor super family of ligand activated transcription factors. Three mammalian Peroxisome Proliferator Activated Receptors (PPARs) have been isolated and termed PPARα, PPARγ and PPARδ. These PPARs are believed to regulate expression of target genes by binding to DNA sequence elements.

Certain PPAR agonist compounds are believed to be useful candidates for treatment of metabolic disorders. See, e.g., U.S. Pat. Nos. 5,885,997, 6,054,453, and U.S. Publication No. 2003/0229083. Nevertheless, there exists a continuing need for new PPAR agonist compounds.

### SUMMARY

In accordance with one aspect, the invention provides a derivative, which is a compound and/or a pharmaceutically acceptable salt of the compound, wherein the compound has the formula (I):

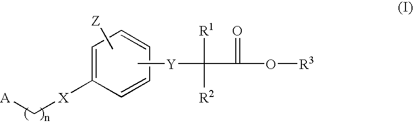

wherein A has the structure

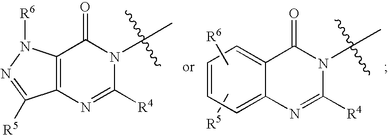

X is chosen from —CH₂—, —O—, —NH—, and —S—; Y is chosen from —O—, —NH—, and —S—; Z, which may be located in any position of substitution, is hydrogen or halogen; R¹ and R², which may be the same or different, are independently chosen from hydrogen and C₁-C₈ alkyl, or R¹ and R² together form a carbocyclic ring having from 4 to 6 carbon atoms; R³ is chosen from hydrogen and C₁-C₈ alkyl; R⁴, R⁵, and R⁶, which may be the same or different, are independently chosen from hydrogen and C₁-C₈ alkyl; and n is 1 to 6. Various embodiments and variants of this aspect of the invention are provided.

In accordance with other aspects, the invention also provides methods of producing a PPARα agonist activity in a mammal, the methods including administering to the mammal an effective amount of certain derivative(s) of the first aspect of the invention, a method of producing a PPARα agonist activity and a PPARγ agonist activity in a mammal, the method including administering to the mammal an effective amount of certain derivative(s); and a pharmaceutical composition that includes the derivative(s) of the first aspect of the invention and one or more pharmaceutically-acceptable excipients. Various embodiments and variants are provided.

### DETAILED DESCRIPTION OF EMBODIMENTS

To describe the invention, certain terms are defined herein as follows.

The use of singular includes the use of plural. In a non-limiting example, a recitation of “a derivative” includes a single derivative, as well as multiple derivatives.

The term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless whether it exists in a free form or and an associated forms.

The term “stereoisomers” is used to refer to both optical isomers and geometrical isomers. A recitation of the chemical structure of the compound encompasses all structural variations possible within the structure as shown.

Thus, some of the described compounds have optical centers. If the optical configuration at a given optical center is not defined with specificity, the recitation of chemical structure covers all optical isomers produced by possible configurations at the optical center. The term “optical isomer” defines a compound having a defined optical configuration at least one optical center. This principle applies for each structural genus described herein, as well as for each subgenus and for individual structures. For example, the recitation of a molecular portion as

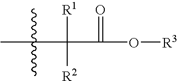

encompasses optical isomers with R and S configurations at the optical center (which arises when R¹ and R² are not identical):

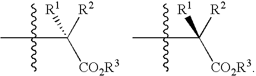

For the purpose of additional illustration, for example, the recitation “a compound of the structure

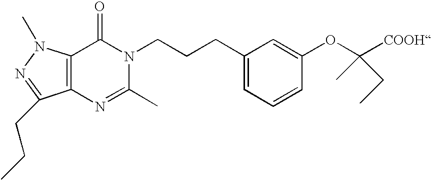

generically encompasses both enantiomers individually:

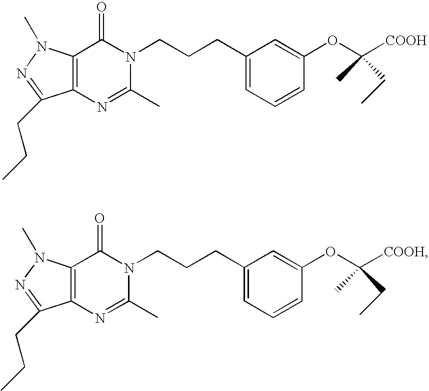

as well as the racemic mixture.

Some of the described compounds may exist as geometrical isomers (e.g., (E), (Z), etc.). If the geometrical configuration is not self-evident from the structure shown, the recitation of the structure generically covers all possible geometrical isomers. This principle applies for each structural genus described herein, as well as for each subgenus and for individual structures.

The compounds may form salts. The term “derivative” is used as a common term for the compound and its salts. Thus, the claim language “a derivative, which is a compound and/or a pharmaceutically-acceptable salt of said compound” is used to define a genus that includes any form of the compound of the given chemical structure and the salts of the recited compound. The use of the term “and/or” is intended to indicate that, for a compound of a given chemical structure, a claim to a “derivative” covers the compound individually, all of its salts individually, and the mixtures of compounds and the salt(s). The term “pharmaceutically-acceptable salts” is intended to denote salts that are suitable for use in human or animal pharmaceutical products. The use of the term “pharmaceutically-acceptable” is not intended to limit the claims to substances (“derivatives”) found only outside of the body.

The term “prodrug” is used to refer to a compound (and/or its salt) capable of converting, either directly or indirectly, into compounds described herein by the action of enzymes, gastric acid and the like under in vivo physiological conditions (e.g., enzymatic oxidation, reduction and/or hydrolysis).

In describing the compounds, certain nomenclature and terminology is used throughout to refer to various groups and substituents. The description “Cₓ-C_{y}” refers to a chain of carbon atoms or a carbocyclic skeleton containing from x to y atoms, inclusive. The designated range of carbon atoms may refer independently to the number of carbon atoms in the chain or the cyclic skeleton, or to the portion of a larger substituent in which the chain or the skeleton is included. For example, the recitation “(C₁-C₅) alkyl” refers to an alkyl group having a carbon chain of 1 to 5 carbon atoms, inclusive of 1 and 5. The chains of carbon atoms of the groups and substituents described and claimed herein may be saturated or unsaturated, straight chain or branched, substituted or unsubstituted.

The term “alkyl,” whether used alone or as a part of another group, is a group or a substituent that includes a chain of carbon atoms. The chains of carbon atoms of the alkyl groups described and claimed herein may be saturated or unsaturated, straight chain or branched, substituted or unsubstituted. In a non-limiting example, “C₁-C₅ alkyl” denotes an alkyl group having carbon chain with from 1 to 5 carbon atoms, inclusive, which carbon may be saturated or unsaturated, straight chain or branched, substituted or unsubstituted.

A “composition” may contain one compound or a mixture of compounds. A “pharmaceutical composition” is any composition useful or potentially useful in producing physiological response in a subject to which such pharmaceutical composition is administered. The term “pharmaceutically acceptable,” with respect to an excipient, is used to define non-toxic substances generally suitable for use in human or animal pharmaceutical products.

As described above, the derivatives of one aspect of the invention include compounds of the formula (I) and their pharmaceutically-acceptable salts:

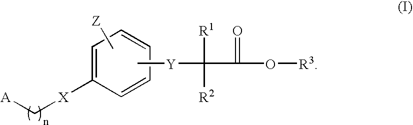

The description of the structure and substitution below applies to the compounds of the formula (I), as well as their variants and embodiments described further. Each group of compounds is separately contemplated. All combinations of substitution and variation of structure are separately contemplated.

### The group A has the structure

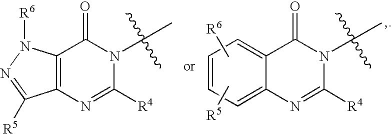

The group X is —CH₂—, —O—, —NH—, or —S—, and the group Y is —O—, —NH—, or —S—. The groups X and Y may be in para- or meta-position to one another, each relative substitution patterns being separately contemplated. Z, which may be hydrogen or halogen, is s substituent of the benzyl ring and may be located in any position of substitution. In one variant that deserve specific mention, when Z is halogen, it may be in the ortho position to the group X. The compounds in which Z is chloro or hydrogen are of specific mention.

R¹ and R², which may be the same or different, are independently chosen from hydrogen and C₁-C₈ alkyl. Of separate mention are compounds in which R¹ and R² are C₁-C₈ alkyl, including methyl, ethyl, i-propyl, and n-propyl. Of separate mention are compounds in which R¹ is methyl and R² is ethyl. Alternatively, R¹ and R² together may form a carbocyclic ring having from 4 to 6 carbon atoms.

R³ is hydrogen or C₁-C₈ alkyl. Of separate mention are compounds in which R³ C₁-C₅ alkyl. Of particular mention are compounds in which R³ is hydrogen. Such compounds, which are carboxylic acids, form carboxylate salts, which are of course separately contemplated.

R⁴, R⁵, and R⁶, which may be the same or different, are independently chosen from hydrogen and C₁-C₈ alkyl. Of separate mention are compounds in which R⁴ is C₁-C₅ alkyl. The compounds in which R⁴ is C₁-C₃ alkyl, including methyl, ethyl, 1-propyl, and n-propyl are separately contemplated; including specifically compounds in which R⁴ is methyl. Of separate mention are compounds in which R⁵ is n-propyl. Also of separate mention are compounds in which R⁶ is methyl. For the compounds of the formula (I), n varies from 1 to 6, inclusive.

Of particular mention are compounds of the formula (I) in which A has the structure

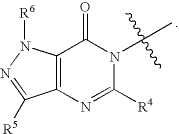

### In one group of these compounds, Y is —O—. Thus, one embodiment provides compounds of the formula (II):

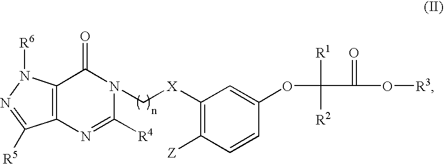

where X is —O— or —CH₂—; Z is hydrogen or chloro; R¹ and R², which may be same or different, are independently chosen from methyl and ethyl; R³ is chosen from hydrogen and C₁-C₅ alkyl; R⁴, R⁵, and R⁶, which may be the same or different, are independently chosen from hydrogen and C₁-C₆ alkyl; and n is 2, 3 or 4.

Of course, all combination of substitutions and variation of structure are separately contemplated. In one variant of the compounds of formula (II), X is —O—. Of particular mention among these compounds are compounds of formula (II), in which X is —O—, R¹ is methyl, R² is ethyl, R⁴, R⁵, and R⁶, which may be the same or different, are independently chosen from C₁-C₆alkyl; and n is 2.

In another variant of the compounds of formula (II), X is —CH₂—. Of particular mention among these compounds are compounds of formula (II), X is —CH₂—, R¹ is methyl, R² is ethyl; R⁴, R⁵, and R⁶, which may be the same or different, are independently chosen from C₁-C₆ alkyl; and n is 2.

Among the compounds in which Y is —O—, another embodiment provides compounds having the formula (III):

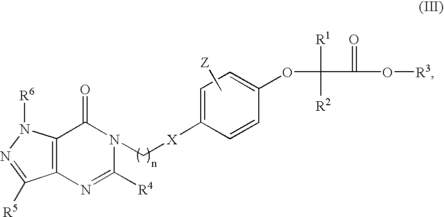

wherein X is —O—, —NH— or —CH₂—; Z is hydrogen or chloro; R¹ and R², which may be same or different, are independently chosen from hydrogen, methyl and ethyl; R³ is chosen from hydrogen and C₁-C₅ alkyl; R⁴, R⁵, and R⁶, which may be the same or different, are independently chosen from hydrogen and C₁-C₆ alkyl; and n is 2, 3 or 4.

Of course, all combination of substitutions and variation of structure are separately contemplated. In one variant of the compounds of formula (III), X is —CH₂—. Of particular mention among these compounds are compounds of formula (III), in which X is —CH₂—, R¹ is methyl, R² is ethyl, R⁴, R⁵, and R⁶, which may be the same or different, are independently chosen from C₁-C₆alkyl; and n is 2.

In another group of compounds of formula (I) in which A has the structure

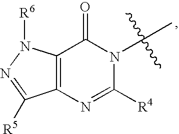

### Y is —S— or —NH—. Thus, one embodiment provides compounds of the formula (IV):

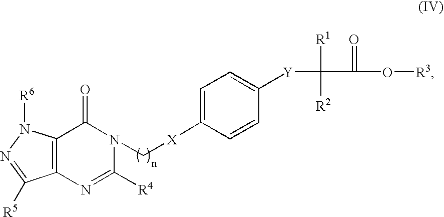

where X is —O— or —NH—; Y is —S— or —NH—; R¹ and R², which may be the same or different, are independently chosen from hydrogen and C₁-C₄ alkyl; R³ is chosen from hydrogen and C₁-C₅ alkyl; R⁴, R⁵, and R⁶, which may be the same or different, are independently chosen from C₁-C₆ alkyl; and n is 2, 3 or 4.

Of course, all combination of substitutions and variation of structure are separately contemplated. In one variant of the compounds of formula (IV), X is —O—. Of particular mention are compounds of the formula (IV), in which X is —O—, R¹, R², R⁴, and R⁶ are independently chosen from C₁-C₄ alkyl.

In another embodiment, there is provided a compound having the formula (V)

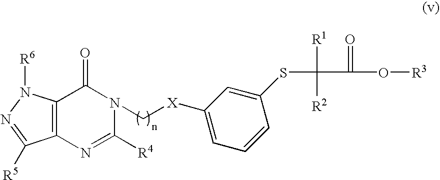

wherein X is —O— or —NH—; R¹ and R², which may be the same or different, are independently chosen from hydrogen and C₁-C₄ alkyl; R³ is chosen from hydrogen and C₁-C₅ alkyl; R⁴, R⁵, and R⁶, which may be the same or different, are independently chosen from C₁-C₆ alkyl; and n is 2, 3 or 4. In one variant of the compounds of formula (V), X is —O—.

As set forth above, the derivatives of the invention include pharmaceutically-acceptable salts of the compounds of the formula (I), including all salt-forming compounds separately discussed. For example, the derivatives of the invention include carboxylic acid salts of compounds in which R³ is hydrogen; which salts are generally prepared by reacting the free acid with a suitable organic or inorganic base.

Examples of salts with inorganic bases include alkali metal salts such as sodium salt and potassium salt, alkaline earth metal salts such as calcium salt and magnesium salt, as well as aluminum salt and ammonium salt. Examples of salts with organic bases include those which are formed with trimethylamine, triethylamine, pyridine, picoline, ethanolamine, diethanolamine, triethanolamine, dicyclohexylamine and N,N′-dibenzylethylenediamine. Examples of salts with inorganic acids include those which are formed with hydrochloric acid, hydrobromic acid, nitric acid, sulfuric acid and phosphoric acid. Examples of salts with organic acids include those which are formed with formic acid, acetic acid, trifluoroacetic acid, fumaric acid, oxalic acid, tartaric acid, maleic acid, citric acid, succinic acid, malic acid, methanesulfonic acid, benzenesulfonic acid and p-toluenesulfonic acid. Examples of salts with basic amino acids include those which are formed with arginine, lysine and ornithine. Examples of salts with acidic amino acids include those which are formed with aspartic acid and glutamic acid.

As set forth above, the compounds described herein encompass any stereoisomers thereof, including optical isomers (e.g., enantiomers) and geometrical isomers. Non-limiting examples of such stereoisomers include (R), (S), a mixture of (R) and (S), (E), (Z) or a mixture of (E) and (Z) or combinations thereof such as (S)(E), (S)(Z), (R)(E), (R)(Z) and the like. The individual optical isomers or required isomers may be obtained by using reagents in such a way to obtain single isomeric form in the process wherever applicable or by conducting the reaction in the presence of reagents or catalysts in their single enantiomeric form. Some of the preferred methods of resolution of racemic compounds include use of microbial resolution, resolving the diastereomeric salts, amides or esters formed with chiral acids such as mandelic acid, camphorsulfonic acid, tartaric acid, lactic acid, and the like wherever applicable or chiral bases such as brucine, cinchona alkaloids and their derivatives and the like. Commonly used methods are compiled by Jaques et al in “Enantiomers, Racemates and Resolution” (Wiley Interscience, 1981), the relevant portion thereof being incorporated by reference herein. Where appropriate, the compounds of formula (I) may be resolved by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula (I) may be prepared by hydrolyzing the pure diastereomeric amide, ester or salt.

The invention also provides prodrugs of the derivatives of the formula (I). Examples of a prodrug of the compound of formula (I) include compounds obtained when an amino group is acylated, alkylated or phosphorylated, such as those obtained when an amino group is eicosanoylated, alanylated, pentylaminocarbonylated, (5-methyl-2-oxo-1,3-dioxolen-4-yl)methoxycarbonylated, tetrahydrofuranylated, tetrahydropyranylated, pyrrolidylmethylated, pivaloyloxymethylated or tert-butylated; compounds obtained when a hydroxy group is acylated, alkylated, phosphorylated or borated, such as those obtained when a hydroxy group is acetylated, palmitoylated, propanoylated, pivaloylated, succinylated, fumarylated, alanylated, dimethylaminomethyl-carbonylated or tetrahydropyranylated; and compounds obtained when a carboxyl group is esterified or amidated, such as those obtained when a carboxyl group is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated. For example, it should be appreciated that the compounds of the formula (I) in which R³ is hydrogen are capable of forming ester prodrugs. Of particular mention are ester prodrugs in which R₃ is not (C₁-C₈) alkyl.

The derivatives described herein are believed to possess at least a baseline level of PPAR agonist activity and as such are useful candidates for use in treating metabolic disorders. Generally, suitable PPAR agonists are believed to be useful for attenuating and/or treatment of diabetic dyslipidemia, metabolic syndrome, diabetes, cardiovascular disease, and obesity.

Peroxisome Proliferator Activated Receptors (PPARs) are members of the nuclear receptor supergene family that play a central role in the regulation of storage and catabolism of dietary fats. The three subtypes of PPAR (designated as α, δ and γ) bind to fatty acids and fatty acid metabolites and regulate the expression of genes involved in the transport, metabolism and buffering of these ligands within cells. Each of the three PPAR subtypes exhibits a unique expression pattern within vertebrate tissues. In rats, PPARα is most highly expressed in brown adipose tissue, followed by liver, kidney, heart and skeletal muscle. PPARγ is most highly expressed in white and brown adipose tissue, but is also expressed in muscle, colon and liver. PPARδ is expressed in all tissues studied to date.

It is believed that PPARα is involved in stimulating beta-oxidation of fatty acids. PPARα is also involved in the control of HDL cholesterol levels in rodents and humans. This effect may at least be partially based on a PPARα mediated transcriptional regulation of the major HDL apolipoproteins, apo A-I and apo A-II. Agonists of PPARα may prevent cardiovascular mortality with fewer adverse effects as they may lower TG and increase HDL levels by activating PPARα.

PPARδ activation was alleged not to be involved in modulation of glucose or triglyceride levels. See, e.g., Berger et al., J. Biol. Chem., 1999, Vol 274, pp. 6718-6725. Some believe that PPARδ activation may lead to increased levels of HDL cholesterol in db/db mice as reported. See, e.g., Leibowitz et al. FEBS letters 2000, 473, 333-336. PCT publication WO 01/00603 alleged that PPARδ activation might be useful in the treatment/prevention of cardiovascular diseases and conditions including arthroscleroses, hypertriglyceridemia, and mixed dyslipidaemia.

Selective activators of PPARγ may be useful for the treatment of NIDDM and other disorders related to lipid metabolism and energy homeostasis. Further, compounds that block PPARγ may useful for interfering with maturation of pre edipo cytes into edipo cytes and thus may be useful treatment of obesity and related disorders associated with undesirable edipocyte maturation.

A number of derivatives of the invention have shown PPAR agonist activity. The following procedure was used for in vitro determination of PPARα, γ and δ transactivation.

The ligand binding domain of human PPARγ1, PPARα or PPARδ was fused to the C-terminal end of DNA binding domain of yeast transcription factor GAL4 in eukaryotic expression vector. HEK-293 cells were transfected with either of these plasmids, reporter plasmid pGL2 (Gal4×5)˜SV40˜Luc and pAdVantage using superfect (Qiagen, Germany) for 3 hours. pAdVantage vector was used to enhance the luciferase expression. 48 hours after transfection, the cells were harvested and incubated overnight with or without test compounds at different concentrations. The cells were lysed and luciferase activity was measured using the LucLite kit (Packard USA). Luciferase activity was measured as fold activation relative to untreated cells.

The following results were obtained:

| Derivative of | | | | |
|---|---|---|---|---|
| Example | Concentration** | | | |
| #* | (μM) | PPARα | PPARδ | PPARγ |
| 82 | 1 | 7.1(0.1) | 0(11.5) | — |
| | 10 | 8.4(2.2) | 0(13.7) | |
| 88 | 1 | 1.6(0.9) | 0.3(20.4) | — |
| | 10 | 3.6(1.7) | 0.6(23.7) | |
| 89 | 1 | 2.1(0.9) | 0(12.5) | 0(54.5) |
| | 10 | 7.4(1.7) | 0.4(14.0) | 0(62.8) |
| 95 | 1 | 6.4(0.9) | 0.1(20.4) | 17.3(85.5) |
| | 10 | 7.3(1.7) | 4.0(23.7) | 36.6(110.3) |
| 98 | 1 | 5.0(1.6) | 0.6(17.3) | 1.1(87.3) |
| | 10 | 5.7(2.5) | 3.3(18.9) | 0.3(85.3) |
| 102 | 1 | 3.9(1.5) | 1.7(10.8) | — |
| | 10 | 5.2(2.2) | 1.3(13.4) | |

*Examples are found at the end of the specification.
**PPARα, PPARδ and PPARγ activations are calculated at 1 μM and 10 μM concentrations with respect to the standards Wyeth 14643, Rosiglitazone and GW 501516 respectively. The values in the parenthesis represent the activations of the standards obtained at given concentrations.

Thus, the derivatives of formula (I), which possess PPARα activity of at least 1.5 at a 1 μM concentration and of at least 4 at a 10 μM concentration as measured by the method described above, are particularly contemplated. In an embodiment, the invention also provides derivatives of the formula (I), which possess PPARα activity of at least 3.5 at a 1 μM concentration and of at least 6 at a 10 μM concentration. In a variant of this embodiment, the invention provides derivatives that possess a PPARγ activity of at least 1.5 at a 1 μM concentration, as measured by the above, in addition to the PPARα activity.

In another aspect, the invention provides a method of producing a PPARα agonist activity in a mammal by administering to the mammal an effective amount of the derivative of the formula (I). A preferred embodiment of this aspect of the invention involves administration of derivatives which possess PPARα activity of at least 1.5 at a 1 μM concentration and of at least 4 at a 10 μM concentration as measured by the method described above.

In yet another aspect, the invention provides a method of producing a PPARα agonist activity and a PPARγ agonist activity in a mammal by administering to the mammal an effective amount of the derivative that possesses PPARα activity of at least 1.5 at a 1 μM concentration and of at least 4 at a 10 μM concentration and PPARγ activity of at least 1.5 at a 1 μM concentration, as measured by the method described above.

The properties of the derivatives of the invention were also evaluated in vivo. The following procedure was used to determine plasma triglyceride and total cholesterol lowering activity in Swiss albino mice.

Male Swiss albino mice (SAM) were obtained from National Institute Nutrition (NIN) and housed in Dr. Reddy's Laboratories Ltd (DRL) animal house. All these animals are maintained under 12 hour light and dark cycle at 25±1° C. Animals were given standard laboratory chow (NIN, Hyderabad, India) and water, ad libitum. SAM of 20-25 grams body weight range were used. See Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice, Atherosclerosis. 1988. 70: 107-114, incorporated herein by reference in its entirety. The test compounds can be administered orally to Swiss albino mice at the dose mentioned in table for 6 days. Control mice were treated with vehicle 0.25% Carboxymethylcellulose (CMC; dose 10 ml/kg). The blood samples from the retro-orbital sinus through heparinised capillary in EDTA containing tubes were collected in fed state 1 hour after drug administration on 0 and 6 days of treatment. After centrifugation plasma was separated for triglyceride measurement using commercial kits. See Wieland, O. Methods of Enzymatic analysis. Bergermeyer, H. O., Ed., 1963. 211-214; Trinder, P. Ann. Clin. Biochem. 1969. 6: 24-27, incorporated herein by reference in its entirety. Percent reduction was calculated as per the formula given in Petit, D., Bonnefis, M. T., Rey, C. and Infante, R. Effects of ciprofibrate on liver lipids and lipoprotein synthesis in normo- and hyperlipidemic rats. Atherosclerosis. 1988. 74 : 215-225.

The following results were obtained in Swiss albino mice:

| Derivative | | |
|---|---|---|
| of Example | | % reduction |
| #* | Dose (mg/kg) | in TG |
| 79 | 3.0 | 55 |
| 82 | 3.0 | 81 |
| 95 | 3.0 | 63 |
| 89 | 3.0 | 60 |
| 96 | 3.0 | 60 |
| 98 | 3.0 | 65 |
| 100 | 3.0 | 76 |
| 102 | 3.0 | 67 |
| 104 | 3.0 | 70 |

*Examples are at the end of the specification

Thus, the derivatives of the formula (I) that reduce the triglyceride levels at least 55%, preferably, 65%, as measured by the procedure described above, are separately contemplated.

The following procedure was used to determine plasma triglyceride and cholesterol lowering activity in hypercholesterolemic rat model.

Male Sprague Dawley rats (NIN stock) were bred in DRL animal house. Animals were maintained under 12 hour light and dark cycle at 25±1° C. Rats of 180-200 gram body weight range were used for the experiment. Animals are made hypercholesterolemic by feeding 2% cholesterol and 1% sodium cholate mixed with standard laboratory chow [National Institute of Nutrition (NIN), Hyderabad, India] for 6 days. Throughout the experimental period the animals were maintained on the same diet. The test compounds were administered orally at a dose mentioned in table for 3 days. Control group was treated with vehicle alone (0.25% CMC; 10 ml/kg). The blood samples were collected in fed state 1 hour after drug administration on 0 and 3 days of compound treatment. The blood was collected from the retro-orbital sinus through heparinised capillary in EDTA containing tubes. After centrifugation, plasma sample was separated for total cholesterol, HDL and triglyceride estimations. Measurement of plasma triglyceride, total cholesterol and HDL were done using commercial kits. LDL and VLDL cholesterol can be calculated from the data obtained for total cholesterol, HDL and triglyceride.

The percent reductions in blood sugar/triglycerides/total cholesterol were calculated according to the following formula:

$$Percent reduction ( \% ) = [ 1 - \frac{TT / OT}{TC / OC} ] \times 100$$

### OC=Zero day control group value

### OT=Zero day treated group value

### TC=Test day control group value

### T=Test day treated group value.

The levels of LDL and VLDL cholesterol were calculated according to the formula:

$$LDL cholesterol in mg \text{/} dl = [ \begin{matrix}Total cholesterol - \\ HDL cholesterol - \\ \frac{Triglyceride}{5}\end{matrix} ] mg \text{/} dl$$

### VLDL cholesterol in mg/dl=[Total cholesterol-HDL cholesterol-LDL cholesterol] mg/dl.

The following results were obtained in hypercholesterolemic rat model.

| Derivative of | Dose | Reduction (%) | | | |
|---|---|---|---|---|---|
| Example #* | (mg/kg) | TC | TG | ↑HDL | LDL |
| 82 | 3.0 | 68 | 69 | 48 | 72 |
| 95 | 3.0 | 64 | 67 | 212 | 69 |
| 89 | 3.0 | 65 | 68 | 102 | 68 |
| 96 | 3.0 | 63 | 76 | 162 | 67 |
| 98 | 3.0 | 67 | 81 | 267 | 75 |
| 100 | 3.0 | 70 | 60 | 145 | 76 |
| 104 | 3.0 | 71 | 72 | 158 | 77 |
| 102 | 3.0 | 51 | 51 | 45 | 55 |

*Examples are at the end of specification.

Thus, methods of using the derivatives of the invention to reduce the levels of LDL cholesterol and triglycerides, and to increase the level HDL cholesterol in mammals, including humans and animals, are also contemplated. Separately contemplated are derivative of the formula (I) that increase the HDL cholesterol level, as measured by the procedure described above, by at least 120%, preferably, by at least 200%.

Compounds of formula (I) can be prepared, for example, in the manner shown in the following preparation schemes.

Compounds of the formula (I) thus prepared may be isolated and purified from the reaction mixture by known means, including solvent extraction, concentration, neutralization, filtration, crystallization, recrystallization, column chromatography, high performance liquid chromatography and recrystallization, to give a highly purified product of interest.

The compounds of the present invention and salts thereof can be prepared by applying various synthetic methods utilizing the characteristics due to the fundamental skeleton or type of the substituents thereof. Representative production methods will be illustrated as hereunder. All other symbols are as defined earlier.

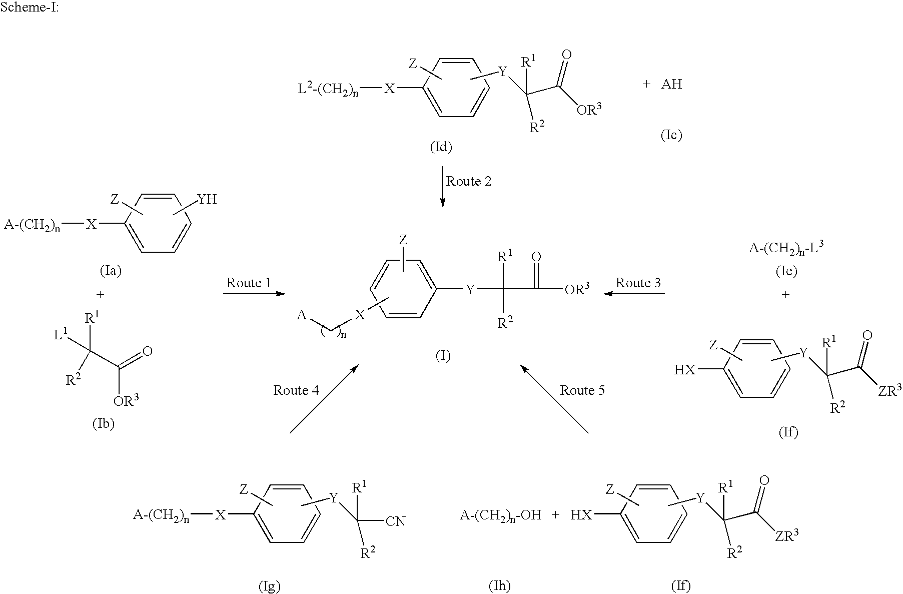

### Route 1:

The reaction of compound of formula (Ia) with a compound of formula (Ib) where all symbols are as defined earlier and L¹ represents a leaving group such as halogen atom, p-toluenesulfonate, methanesulfonate, trifluoromethane sulfonate and the like, to produce a compound of the formula (I), may be carried out in the presence of aprotic solvents such as toluene, benzene, xylene, tetrahydrofuran, dimethylformamide, dimethylsulphoxide, dimethoxyethane and the like. The reaction may be carried out in an inert atmosphere that can be maintained by using inert gases such as nitrogen, argon, helium and the like. The reaction may be carried out in the presence of a base such as alkalis like sodium hydroxide, potassium hydroxide and the like; alkali metal carbonates such as sodium carbonate, potassium carbonate and the like; alkali metal hydrides such as sodium hydride, potassium hydride and the like; organometallic bases like n-butyl lithium, lithium diisopropyl-amide and the like; alkali metal amides like sodamire, organic base like triethyl amine, lutidine, collidine and the like or mixtures thereof. Acetone may be used as solvent when alkali metal carbonate is used as a base. Phase transfer catalyst such as tetraalkyl ammoniumhalide, hydrogensulphate and the like, may be added. Additives such as alkali metal halides like lithiumbromide may be added. The reaction temperature may range from 0° C. to about 160° C., preferably at a temperature in the range of about 25 to about 100° C. The duration of the reaction may range from about 1 to about 120 hours, preferably from about 2 to about 24 hours.

### Route 2:

The reaction of a compound of general formula (Ic) where A is as defined earlier with a compound of general formula (Id) where L² is a leaving group such as halogen atom, p-toluenesulfonate, methanesulfonate, trifluoromethane sulfonate and the like, and all other symbols are as defined earlier, to produce a compound of general formula (I) where all symbols are as defined earlier, may be carried out in the presence of solvents such as toluene, benzene, xylene, dimethylsulphoxide, dimethylformamide, dimethoxyethane, tetrahydrofuran, dioxane, ether, acetone and the like or mixtures thereof. The reaction may be carried out in an inert atmosphere that can be maintained by using inert gases such as nitrogen, argon, helium and the like. The reaction may be effected in the presence of a base such as alkalis like sodium hydroxide, potassium hydroxide and the like; alkali metal carbonates such as sodium carbonate, potassium carbonate and the like; alkali metal hydrides such as sodium hydride, potassium hydride and the like; organometallic bases like n-butyl lithium, lithium diisopropyl-amide and the like; alkali metal amides like sodamide, organic base like triethyl amine, lutidine, collidine and the like or mixtures thereof. The amount of base may range from 1 to 5 equivalents, based on the amount of the compound of formula (Id), preferably the amount of base ranges from 1 to 3 equivalents. Phase transfer catalysts such as tetraalkylammonium halide, hydrogensulphate or hydroxide may be added. Additives such as alkali metal halides like lithiumbromide may be added. The reaction may be carried out at a temperature in the range of 0° C. to about 160° C., preferably at a temperature in the range of about 15 to about 100° C. The duration of the reaction may range from about 15 minutes to about 120 hours, preferably from about 15 minutes to about 24 hours.

### Route 3:

The reaction of compound of formula (Ie) where L³ represents a leaving group such as halogen atom, p-toluenesulfonate, methanesulfonate, trifluoro methanesulfonate and the like with a compound of formula (If) where all symbols are as defined earlier to produce a compound of the formula (I), may be carried out in the presence of aprotic solvents such as toluene, benzene, xylene, dimethylsulphoxide, dimethylformamide, dimethoxy ethane, tetrahydrofuran, acetone and the like or mixtures thereof. The reaction may be carried out in an inert atmosphere that can be maintained by using inert gases such as nitrogen, argon, helium and the like. The reaction may be effected in the presence of a base like alkali metal corbonate such as potassium carbonate, sodium carbonate and the like; alkali metal hydrides like sodium hydride, potassium hydride and the like; triethyl amine and the like or mixtures thereof. Acetone may be used as solvent when alkali metal carbonate is used as a base. Phase transfer catalyst such as tetraalkylammonium halide, hydrogensulphate and the like, may be added. Additives such as alkali metal halides like lithiumbromide may be added. The reaction temperature may range from 0° C. to about 160° C., preferably at a temperature in the range of about 25 to about 100° C. The duration of the reaction may range from about 1 to about 120 hours, preferably from about 2 to about 48 hours.

### Route 4:

The conversion of compound of formula (Ig) to a compound of formula (I) may be carried out either in the presence of base or an acid and the selection of base or acid is not critical. Any base normally used for hydrolysis of nitrile to acid may be employed, metal hydroxides such as sodium hydroxide, potassium hydroxide and the like, in an aqueous solvent or any acid normally used for hydrolysis of nitrile may be employed such as hydrochloric acid in an excess of alcohol such as methanol, ethanol, propanol and the like. The reaction may be carried out at a temperature in the range of 0° C. to reflux temperature of the solvent used, preferably at a temperature in the range of about 25° C. to reflux temperature of the solvent used. The duration of the reaction may range from about 0.25 to about 96 hours.

### Route 5:

The reaction of compound of general formula (Ih) with a compound of general formula (If) where all symbols are as defined earlier, may be carried out using suitable coupling agents such as 1-(3-dimethylaminopropyl)-3-ethylcarbodiimide hydrochloride, dicyclohexyl urea, triarylphosphine/dialkylazadicarboxylate such as triphenylphosphine/diethyl azodicarboxylate or diisopropyl azodicarboxylate and the like. The reaction may be carried out in the presence of solvents such as dimethoxyethane, tetrahydrofuran, dichloromethane, chloroform, toluene, acetonitrile, carbon tetrachloride and the like. The inert atmosphere can be maintained by using inert gases such as nitrogen, argon, helium and the like. The reaction may be effected in the presence of 4-dimethylaminopyridine, 1-hydroxybenzotriazole, triethylamine and they may be used in the range of 0.05 to 2 equivalents, preferably 0.25 to 1 equivalents. The reaction temperature may be in the range of about −20 to about 100° C., preferably at a temperature in the range of 0° C. to about 80° C. The duration of the reaction may range from about 0.5 to about 48 hours, preferably from about 6 to about 18 hours. The above condensation may also be made using mixed anhydride methodology.

In another aspect, the invention also provides pharmaceutical compositions that include the derivative of the formula (I) and one or more pharmaceutically-acceptable excipients. The pharmaceutical compositions are prepared by admixture and are suitably adapted for oral, parenteral or topical administration, and as such may be in the form of tablets, capsules, oral liquid preparations, powders, granules, lozenges, pastilles, reconstitutable powders, injectable and infusible solutions or suspensions, suppositories and transdermal devices. The compound(s) of the formula (I) as defined above is clinically administered to mammals, including man, via either oral or parenteral routes. Administration by the oral route is preferred being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.01 to about 100 mg/kg body weight of the subject per day or preferably about 0.01 to about 50 mg/kg body weight per day administered singly or as a divided dose. However, the optimum dosage for the individual subject being treated will be determined by the person responsible for treatment, generally smaller doses being administered initially and thereafter increments made to determine the most suitable dosage.

Tablets and capsules for oral administration are usually presented in a unit dose, and contain conventional excipients such as binding agents, fillers, diluents, tableting agents, lubricants, disintegrants, colorants, flavorings, and wetting agents. The tablets may be coated according to methods known in the art.

Suitable fillers for use include cellulose, mannitol, lactose and other similar agents. Suitable disintegrants include starch, polyvinylpyrrolidone and starch derivatives such as sodium starch glycolate. Suitable lubricants include, for example, magnesium stearate. Suitable pharmaceutically acceptable wetting agents include sodium lauryl toluenesulfonate.

Solid oral compositions may be prepared by conventional methods of blending, filling, tableting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminum stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the active compound in a vehicle and filter sterilizing before filling into a suitable vial or ampoule and sealing. Advantageously, adjuvants such as a local anesthetic, preservatives and buffering agents are also dissolved in the vehicle. To enhance the stability, the composition can be frozen after filling into the vial and the water removed under vacuum.

Parenteral suspensions are prepared in substantially the same manner except that the active compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound.

The invention is further illustrated with reference to the following examples, none of which is of course limiting.

### PREPARATION 1

### N1-(2-Hydroxyethyl)-2-aminobenzamide

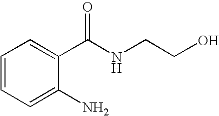

2-Amino-ethanol (13.33 grams, 213.87 mmol) was added drop wise to a solution of 1H-benzo[d][1,3]oxazin-2,4-dione (30 grams, 178.38 mmol) in 1,4-dioxane (300 mL) and stirred at 20 to 40° C. for 4 hours. Reaction mixture was then concentrated and traces of 1,4-dioxane were removed azeotropically by using benzene (3×100 mL). The solid product was then dried under vacuum to yield the title compound. Yield: 29 grams, 87.6%. Melting Point: 89-90° C. ¹H NMR (DMSO-d₆, 300 MHz): δ 2.59 (t, J=5.7 Hz, 1H, D₂O exchangeable), 3.27 (q, J=6.0 Hz, 2H), 3.37 (t, J=5.7 Hz, 2H), 6.37 (s, 2H, D₂O exchangeable), 6.49 (t, J=8.0 Hz, 1H), 6.67 (dd, J=0.7 & 8.2 Hz, 1H), 7.11 (t, J=8.3 Hz, 1H), 7.48 (dd, J=1.2 & 7.9 Hz, 1H), 8.14 (t, J=5.2 Hz, 1H, D₂O exchangeable).

### PREPARATION 2

### 2-(2-Methyl-4-oxo-3,4-dihydro-3-quinazolinyl)ethyl acetate

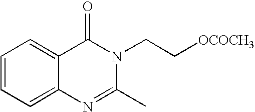

Acetyl chloride (7.85 grams, 9.8 mmol) was added drop wise in 30 minutes duration to a mixture of N1-(2-hydroxyethyl)-2-aminobenzamide (9 grams, 49 mmol), obtained in preparation 1, in xylene (30 mL) and triethyl amine (19.81 grams, 192.1 mmol) at 0-5° C. and reaction mixture was stirred at 20 to 40° C. for 4 hours. Acetic acid (18 mL) was then added and the mixture was heated to 167° C. for 48 hours. The reaction mixture was filtered and washed with ethyl acetate (3×50 mL) and solvents were removed under reduced pressure at 60° C. The compound was purified by chromatography on neutral alumina column using 35% ethyl acetate in hexane to yield the title compound. Yield: 4 grams, 32.5%. ¹H NMR (CDCl₃, 300 MHz): δ 2.06 (s, 3H), 2.7 (s, 3H), 4.35-4.44 (m, 4H), 7.45 (t, J=7.5 Hz, 1H), 7.62 (d, J=8.1 Hz, 1H), 7.70-7.77 (m, 1H), 8.24 (dd, J=8.0 Hz, 1H).

### PREPARATION 3

### 3-(2-Hydroxyethyl)-2-methyl-3,4-dihydro-4-quinazolinone

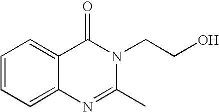

To a solution of 2-(2-methyl-4-oxo-3,4-dihydro-3-quinazolinyl)ethyl acetate (2 grams, 7.7 mmol), obtained in preparation 2, in methanol (20 mL) sodium carbonate (1.63 grams, 15.3 mmol) in water (6 mL) was added drop wise. Reaction mixture was then stirred at 20 to 40° C. for 4 hours. The reaction mixture was filtered, washed thrice with ethyl acetate (3×10 mL). The aqueous layer was concentrated and the residue was diluted with water (200 mL). The aqueous layer was extracted with ethyl acetate (3×63 mL). The combined organic layers was dried over anhydrous sodium sulfate. The product was concentrated to give title compound. Yield: 1.66 grams, 96.4%. Melting Point: 158-160° C. ¹H NMR (CDCl₃, 300 MHz): δ 2.71 (s, 3H), 4.03 (t, J=5.2 Hz, 2H), 4.30 (t, J=5.2 Hz, 2H), 7.4 (t, J=8.1 Hz, 1H), 7.55 (d, J=7.8 Hz, 1H), 7.65-7.74 (m, 1H), 8.13 (dd, J=1.1 & 8.0 Hz, 1H).

### PREPARATION 4

### 3-(2-Chloroethyl)-2-methyl-3,4-dihydro-4-quinazolinone

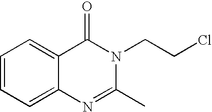

To a solution of 3-(2-hydroxyethyl)-2-methyl-3,4-dihydro-4-quinazolinone (1.4 grams, 6.7 mmol), obtained in preparation 3, in dichloromethane (14 mL) and triethyl amine (3.39 grams, 33.16 mmol) methanesulfonylchloride was added drop wise at 0-5° C. Reaction mixture was stirred at 20 to 40° C. for 2 hours. Excess of dichloromethane (˜50 mL) was added and concentrated to remove traces of methane sulfonyl chloride. The organic layer was washed with water (50 mL), saturated sodium bicarbonate (50 mL) and brine (50 mL). The organic layer was, dried over sodium sulfate and concentrated to obtain the title compound. Yield: 1.35 grams, 88.8%. Melting Point: 124-126° C. ¹H NMR (CDCl₃, 300 MHz): δ 2.73 (s, 3H), 3.91 (t, J=6.2 Hz, 2H), 4.44 (t, J=6.3 Hz, 2H), 7.46 (t, J=8.0 Hz, 1H), 7.63 (d, J=7.8 Hz, 1H), 7.7-7.79 (m, 1H), 8.24 (dd, J=1.1 & 8.0 Hz, 1H).

### PREPARATION 5

### 2-(2-Ethyl-4-oxo-3,4-dihydro-3-quinazolinyl)ethyl propanoate

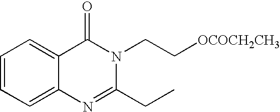

The title compound was prepared by following the same procedure as described in the preparation 2, by heating N1-(2-hydroxyethyl)-2-aminobenzamide (12 grams, 65.3 mmol), obtained in preparation 1, triethyl amine (23.55 grams, 228.5 mmol), propionic acid (24 mL) and propionyl chloride (15.41 grams, 163.3 mmol) in xylene (120 mL) at 167° C. for 48 hours. Yield: 10.5 grams, 58%. ¹H NMR (CDCl₃, 300 MHz): δ 1.09 (t, J=7.6 Hz, 3H), 1.39 (t, J=7.4 Hz, 3H), 2.3 (q, J=7.5 Hz, 2H), 2.92 (q, J=7.4 Hz, 2H), 4.36-4.42 (m, 4H), 7.42 (t, J=8.1 Hz, 1H), 7.63 (dd, J=0.7 & 8.1 Hz, 1H), 7.67-7.75 (m, 1H), 8.23 (dd, J=1.0 & 8.0 Hz, 1H).

### PREPARATION 6

### 2-Ethyl-3-(2-hydroxyethyl)-3,4-dihydro-4-quinazolinone

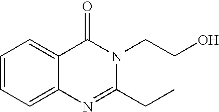

The title compound was prepared by following the same procedure as described in the preparation 3, by treating 2-(2-ethyl-4-oxo-3,4-dihydro-3-quinazolinyl)ethyl propanoate (10.5 grams, 37.0 mmol), obtained in preparation 5, with sodium carbonate (7.96 grams, 74.7 mmol) in methanol-water (130 mL, 10:3) at 20 to 40° C. for 4 hours.

Yield: 8.3 grams, 99%. Melting Point: 140-141° C. ¹H NMR (CDCl₃, 300 MHz): δ 1.39 (t, J=7.2 Hz, 3H), 2.96 (q, J=7.3 Hz, 2H), 4.0 (t, J=5.3 Hz, 2H), 4.32 (t, J=5.3 Hz, 2H), 7.42 (t, J=8.1 Hz, 1H), 7.63 (d, J=7.6 Hz, 1H), 7.69-7.75 (m, 1H), 8.18 (dd, J=1.1 & 8.0 Hz, 1H).

### PREPARATION 7

### 3-(2-Chloroethyl)-2-ethyl-3,4-dihydro-4-quinazolinone

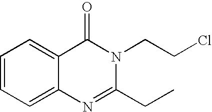

The title compound was prepared by following the same procedure as described in the preparation 4, by using 2-ethyl-3-(2-hydroxyethyl)-3,4-dihydro-4-quinazolinone (3 grams, 13.4 mmol), obtained in preparation 6 and thionyl chloride (8.02 grams, 66.75 mmol) in dichloromethane (30 mL) at 20 to 40° C. for 12 to 17 hours. Yield: 3.15 grams, 96.8%. Melting Point: 126-129° C. ¹H NMR (CDCl₃, 300 MHz): δ 1.41 (t, J=7.4 Hz, 3H), 2.97 (q, J=7.4 Hz, 2H), 3.88 (t, J=6.5 Hz, 2H), 4.44 (t, J=6.5 Hz, 2H), 7.41-7.48 (m, 1H), 7.66 (dd, J=0.7 & 8.2 Hz, 1H), 7.71-7.78 (m, 1H), 8.23 (dd, J=1.0 & 7.9 Hz, 1H).

### PREPARATION 8

### 2-(4-Oxo-2-propyl-3,4-dihydro-3-quinazolinyl)ethyl butyrate

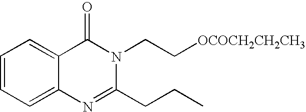

The title compound was prepared by following the same procedure as described in the preparation 2, by heating N-1-(2-hydroxyethyl)-2-aminobenzamide (12 grams, 65.3 mmol), obtained in preparation 1, triethyl amine (23.55 grams, 228.5 mmol), butyric acid (24 mL) and butyryl chloride (17.74 grams, 163.3 mmol) in xylene (120 mL) at 167° C. for 48 hours. Yield: 11.0 grams, 54.6%.

¹H NMR (CDCl₃, 300 MHz): δ 0.91 (t, J=7.4 Hz, 3H), 1.10 (t, J=7.4 Hz, 3H), 1.55-1.68 (m, 2H), 1.81-1.94 (m, 2H), 2.28 (t, J=7.4 Hz, 2H), 2.84-2.90 (m, 2H), 4.38-4.47 (m, 4H), 7.41-7.47 (m, 1H), 7.62-7.67 (m, 1H), 7.70-7.76 (m, 1H), 8.25 (dd, J=1.5 & 8.0 Hz, 1H).

### PREPARATION 9

### 3-(2-Hydroxyethyl)-2-propyl-3,4-dihydro-4-quinazolinone

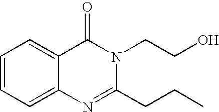

The title compound was prepared by following the same procedure as described in the preparation 3, by using 2-(4-oxo-2-propyl-3,4-dihydro-3-quinazolinyl)ethyl butyrate (11 grams, 35.69 mmol), obtained in preparation 8, and sodium carbonate (7.5 grams, 69.3 mmol) in methanol-water (143 mL, 11:3.3) for 4 hours at 20 to 40° C. Yield: 8.3 grams, 98.8%.

¹H NMR (CDCl₃, 300 MHz): δ 1.08 (t, J=7.4 Hz, 3H), 1.79-1.91 (m, 2H), 2.9 (t, J=7.8 Hz, 2H), 4.0 (t, J=5.3 Hz, 2H), 4.31 (t, J=5.3 Hz, 2H), 7.4 (t, J=8.1 Hz, 1H), 7.6 (d, J=8.1 Hz, 1H), 7.67-7.73 (m, 1H), 8.16 (dd, J=1.1 & 8.0 Hz, 1H).

### PREPARATION 10

### 3-(2-Chloroethyl)-2-propyl-3,4-dihydro-4-quinazolinone

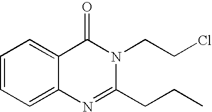

The title compound was prepared by following the same procedure as described in the preparation 4, by stirring 3-(2-hydroxyethyl)-2-propyl-3,4-dihydro-4-quinazolinone (7.2 grams, 30.41 mmol), obtained in preparation 9, and thionyl chloride (17.0 grams, 141.3 mmol) in dichloromethane (72 mL) at 20 to 40° C. for 8 hours.

Yield: 7.3 grams, 94%. ¹H NMR (CDCl₃, 300 MHz): δ 1.04 (t, J=7.4 Hz, 3H), 1.75-1.88 (m, 2H), 2.87 (t, J=7.7 Hz, 2H), 3.82 (t, J=6.4 Hz, 2H), 4.37 (t, J=6.4 Hz, 2H), 7.39 (t, J=8.1 Hz, 1H), 7.60-7.71 (m, 2H), 8.15-8.19 (m, 1H).

### PREPARATION 11

### N1-(3-Hydroxypropyl)-2-aminobenzamide

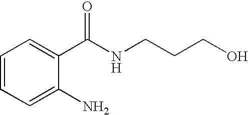

The title compound was prepared by following the same procedure as described in the preparation 1, by treating 1H-benzo[d][1,3]oxazine-2,4-dione (10 grams, 60.1 mmol) with 3-amino-1-propanol (5.41 grams, 70.64 mmol) in 1,4-dioxane (100 mL) at 20 to 40° C. for 4 hours. Yield: 11.3 grams, 96.5%.

¹H NMR (CD₃OD, 300 MHz): δ 1.79-1.89 (m, 2H), 3.46 (t, J=6.9 Hz, 2H), 3.68 (t, J=6.3 Hz, 2H), 6.65 (t, J=8.1 Hz, 1H), 6.77 (d, J=8.2 Hz, 1H), 7.20 (t, J=8.4 Hz, 1H), 7.44 (dd, J=1.3 & 7.9 Hz, 1H).

### PREPARATION 12

### 3-(2-Methyl-4-oxo-3,4-dihydro-3-quinazolinyl)propyl acetate

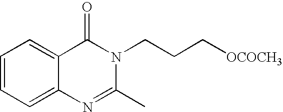

The title compound was prepared by following the same procedure as described in the preparation 2, by heating N1-(3-hydroxypropyl)-2-aminobenzamide (10 grams, 60.43 mmol), obtained in preparation 11, triethylamine (41.6 grams, 403.4 mmol), acetic acid (20 mL) and acetyl chloride (12.1 grams, 151.05 mmol) in xylene (100 mL) at 167° C. for 48 hours. Yield: 3.9 grams, 29.3%. ¹H NMR (CDCl₃, 300 MHz): δ 2.06 (s, 3H), 2.16-2.32 (m, 2H), 2.68 (s, 3H), 4.19-4.24 (m, 4H), 7.45 (t, J=8.1 Hz, 1H), 7.63 (d, J=7.6 Hz, 1H), 7.70-7.77 (m, 1H), 8.25 (dd, J=1.1 & 8.0 Hz, 1H).

### PREPARATION 13

### 3-(3-Hydroxypropyl)-2-methyl-4-oxo-3,4-dihydro-3-quinazolinone

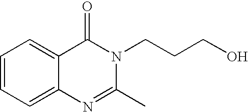

The title compound was prepared by following the same procedure as described in the preparation 3, by using 3-(2-methyl-4-oxo-3,4-dihydro-3-quinazolinyl)propyl acetate (4.4 grams, 16.1 mmol), obtained in preparation 12, and sodium carbonate (3.47 grams, 32.1 mmol) in methanol-water (60 mL, 3:1) at 20 to 40° C. for 4 hours. Yield: 2.5 grams, 78.1%.

¹H NMR (CDCl₃, 300 MHz): δ 1.94-2.02 (m, 2H), 2.70 (s, 3H), 3.31 (s, 1H, D₂O exchangeable), 3.57-3.65 (m, 2H), 4.31 (t, J=6.5 Hz, 2H), 7.47 (t, J=8.0 Hz, 1H), 7.63 (d, J=7.8 Hz, 1H), 7.72-7.78 (m, 1H), 8.26 (dd, J=1.2 & 8.0 Hz, 1H).

### PREPARATION 14

### 3-(3-Chloropropyl)-2-methyl-3,4-dihydro-4-quinazolinone

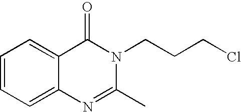

The title compound was prepared by following the same procedure as described in the preparation 4, by using 3-(3-hydroxypropyl)-2-methyl-4-oxo-3,4-dihydro-3-quinazolinone (2.2 grams, 9.8 mmol), obtained in preparation 13, and thionyl chloride (5.87 grams, 48.8 mmol) in dichloromethane (22 mL) at 20 to 40° C. for 8 hours.

Yield: 2.3 grams, 96.6%.

¹H NMR (CDCl₃, 300 MHz): δ 2.18-2.27 (m, 2H), 2.68 (s, 3H), 3.37 (t, J=6.1 Hz, 2H), 4.26 (t, J=7.6 Hz, 2H), 7.43 (t, J=8.1 Hz, 1H), 7.6 (d, J=7.6 Hz, 1H), 7.68-7.74 (m, 1H), 8.22 (dd, J=1.1 & 8.0 Hz, 1H).

### PREPARATION 15

### 3-(2-Ethyl-4-oxo-3,4-dihydro-4-quinazolinyl)propyl propionate

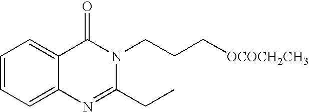

The title compound was prepared by following the same procedure as described in the preparation 2, by heating N1-(3-hydroxypropyl)-2-aminobenzamide (8 grams, 39.07 mmol), obtained in preparation 11, triethylamine (13.81 grams, 135.1 mmol), propionic acid (16 mL) and propionyl chloride (14.38 mL, 168.5 mmol) in xylene (100 mL) at 167° C. for 70 hours. Yield: 3.9 grams, 11.9%.

¹H NMR (CDCl₃, 300 MHz): δ 1.15 (t, J=7.5 Hz, 3H), 1.43 (t, J=7.4 Hz, 3H), 2.1-2.3 (m, 2H), 2.34 (q, J=7.6 Hz, 2H), 2.89 (q, J=7.4 Hz, 2H), 4.19-4.25 (m, 4H), 7.42-7.48 (m, 1H), 7.66 (dd, J=0.8 & 8.1 Hz, 1H), 7.70-7.76 (m, 1H), 8.25 (dd, J=1.0 & 8.0 Hz, 1H).

### PREPARATION 16

### 2-Ethyl-3-(3-hydroxypropyl)-3,4-dihydro-4-quinazolinone

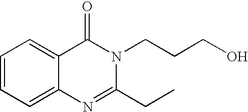

The title compound was prepared by following the same procedure as described in the preparation 3, by stirring 3-(2-ethyl-4-oxo-3,4-dihydro-4-quinazolinyl)propyl propionate (3.9 grams, 13.2 mmol), obtained in preparation 15, and sodium carbonate (2.81 grams, 26 mmol) in methanol-water (52 mL, 3:1) at 20 to 40° C. for 4 hours. Yield: 2.9 grams, 92.3%.

¹H NMR (CDCl₃, 300 MHz): δ 1.44 (t, J=7.3 Hz, 3H), 1.93-2.01 (m, 2H), 2.91 (q, J=7.3 Hz, 2H), 3.32 (t, J=6.0 Hz, 1H, D₂O exchangeable), 3.6 (q, J=6.0 Hz, 2H), 4.32 (t, J=6.4 Hz, 2H), 7.46 (t, J=8.1 Hz, 1H), 7.67 (d, J=7.0 Hz, 1H), 7.72-7.78 (m, 1H), 8.27 (dd, J=1.4 & 8.0 Hz, 1H).

### PREPARATION 17

### 3-(3-Chloropropyl)-2-ethyl-3,4-dihydro-4-quinazolinone

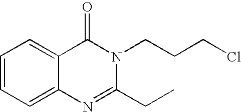

The title compound was prepared by following the same procedure as described in the preparation 4, by using 2-ethyl-3-(3-hydroxypropyl)-3,4-dihydro-4-quinazolinone (2.9 grams, 12.2 mmol), obtained in preparation 16, and thionyl chloride (7.27 grams, 60.4 mmol) in dichloromethane (29 mL) at 20 to 40° C. for 12 to 17 hours.

Yield: 3.0 grams, 96%.

¹H NMR (CDCl₃, 300 MHz): δ 1.4 (t, 3H), 1.9 (m, 2H), 2.9 (q, 2H), 3.6 (q, 2H), 4.3 (t, 2H), 7.4 (t, 1H), 7.6 (d, 1H), 7.7 (t, 1H), 8.2 (d, 1H).

### PREPARATION 18

### 3-(4-Oxo-2-propyl-3,4-dihydro-4-quinazolinyl)propyl butyrate

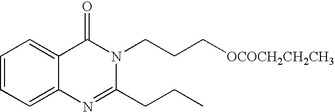

The title compound was prepared by following the same procedure as described in the preparation 2, by heating N1-(3-hydroxypropyl)-2-aminobenzamide (10 grams, 49.47 mmol), obtained in preparation 11, triethylamine (17.6 grams, 171.1 mmol), butyric acid (20 mL) and butyryl chloride (13.17 grams, 123.0 mmol) in xylene (100 mL) at 167° C. for 72 hours. Yield: 3.5 grams, 21.3%.

### PREPARATION 19

### 3-(3-Hydroxypropyl)-2-propyl-3,4-dihydro-4-quinazolinone

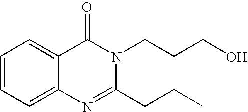

The title compound was prepared by following the same procedure as described in the preparation 3, by treating 3-(4-oxo-2-propyl-3,4-dihydro-4-quinazolinyl)propyl butyrate (3.5 grams, 10.85 mmol), obtained in preparation 18, with sodium carbonate (2.3 grams, 21.26 mmol) in methanol-water (44 mL, 4:1) for 4 hours at 20 to 40° C.

Yield: 2.2 grams, 80.9%.

¹H NMR (CDCl₃, 300 MHz): δ 1.09 (t, J=7.4 Hz, 3H), 1.84-2.00 (m, 4H), 2.83 (t, J=7.7 Hz, 2H), 3.59 (t, J=5.5 Hz, 2H), 4.31 (t, J=6.4 Hz, 2H), 7.42-7.48 (m, 1H), 7.64 (dd, J=0.6 & 8.2 Hz, 1H), 7.71-7.77 (m, 1H), 8.25 (dd, J=1.1 & 8.0 Hz, 1H).

### PREPARATION 20

### 3-(3-Chloropropyl)-2-propyl-3,4-dihydro-4-quinazolinone

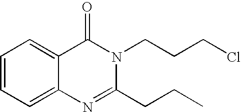

The title compound was prepared by following the same procedure as described in the preparation 4, by using 3-(3-hydroxypropyl)-2-propyl-3,4-dihydro-4-quinazolinone (2.2 grams, 8.76 mmol), obtained in preparation 19, and thionyl chloride (5.21 grams, 43.4 mmol) in dichloromethane (22 mL) at 20 to 40° C. for 12 to 17 hours. Yield: 2.2 grams, 95.6%.

¹H NMR (CDCl₃, 300 MHz): δ 1.11 (t, J=7.3 Hz, 3H), 1.84-1.96 (m, 2H), 2.19-2.28 (m, 2H), 2.85 (t, J=7.6 Hz, 2H), 3.69 (t, J=6.0 Hz, 2H), 4.27 (t, J=7.4 Hz, 2H), 7.44 (t, J=8.1 Hz, 1H), 7.64 (d, J=8.1 Hz, 1H), 7.69-7.75 (m, 1H), 8.24 (dd, J=1.4 & 8.0 Hz, 1H).

### PREPARATION 21

### N1-(4-Hydroxybutyl)-2-aminobenzamide

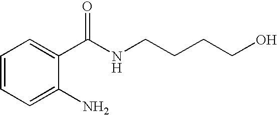

The title compound was prepared by following the same procedure as described in the preparation 1, by treating 1H-benzo[d][1,3]oxazin-2,4-dione (8 grams, 163.1 mmol) with 4-amino-1-butanol (5.14 grams, 56.5 mmol) in 1,4-dioxane (80 mL) at 20 to 40° C. for 4 hours. Yield: 10.2 grams, 98%.

¹H NMR (CD₃OD, 300 MHz): δ 1.60-1.70 (m, 4H), 3.38 (t, J=6.8 Hz, 2H), 3.63 (t, J=6.2 Hz, 2H), 6.62-6.68 (m, 1H), 6.77 (dd, J=0.8 & 8.2 Hz, 1H), 7.17-7.23 (m, 1H), 7.45 (dd, J=1.4 & 7.9 Hz, 1H).

### PREPARATION 22

### 4-(2-Methyl-4-oxo-3,4-dihydro-3-quinazolinyl)butyl acetate

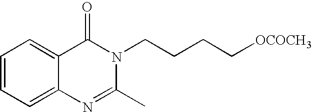

The title compound was prepared by following the same procedure as described in the preparation 2, by heating N1-(4-hydroxybutyl)-2-aminobenzamide (12 grams, 56.46 mmol), obtained in preparation 21, triethylamine (19.96 grams, 193.67 mmol), acetic acid (24 mL) and acetyl chloride (11 grams, 138.2 mmol) in xylene (120 mL) at 167° C. for 48 hours. Yield: 8 grams, 50.6%.

¹H NMR (CDCl₃, 300 MHz): δ 1.68-1.78 (m, 4H), 1.99 (s, 3H), 2.59 (s, 3H), 4.04-4.10 (m, 4H), 7.37 (t, J=8.1 Hz, 1H), 7.54 (d, J=8.1 Hz, 1H), 7.62-7.68 (m, 1H), 8.18 (dd, J=1.1 & 8.0 Hz, 1H).

### PREPARATION 23

### 3-(4-Hydroxybutyl)-2-methyl-3,4-dihydro-4-quinazolinone

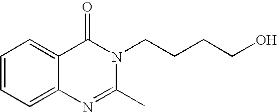

The title compound was prepared by following the same procedure as described in the preparation 3, by using 4-(2-methyl-4-oxo-3,4-dihydro-3-quinazolinyl)butyl acetate (8 grams, 28.6 mmol), obtained in preparation 22, and sodium carbonate (6.18 grams, 57.2 mmol) in methanol-water (104 mL, 10:3) at 20 to 40° C. for 4 hours. Yield: 6.7 grams, 97.4%.

¹H NMR (CDCl₃, 300 MHz): δ 1.64-1.73 (m, 2H), 1.79-1.88 (m, 2H), 2.65 (s, 3H), 3.74 (t, J=6.1 Hz, 2H), 4.10-4.15 (m, 2H), 7.41 (t, J=8.1 Hz, 1H), 7.59 (d, J=8.1 Hz, 1H), 7.67-7.73 (m, 1H), 8.21 (dd, J=1.0 & 8.1 Hz, 1H).

### PREPARATION 24

### 3-(4-Chlorobutyl)-2-methyl-3,4-dihydro-4-quinazolinone

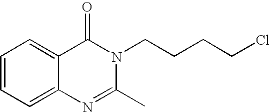

The title compound was prepared by following the same procedure as described in the preparation 4, by treating 3-(4-hydroxybutyl)-2-methyl-3,4-dihydro-4-quinazolinone (6.6 grams, 27.8 mmol), obtained in preparation 23, and thionyl chloride (16.56 grams, 136.4 mmol) in dichloromethane (60 mL) at 20 to 40° C. for 8 hours.

Yield: 7 grams, 98.5%.

### PREPARATION 25

### 4-(2-Ethyl-4-oxo-3,4-dihydro-3-quinazolinyl)butyl propionate

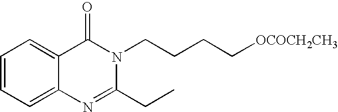

The title compound was prepared by following the same procedure as described in the preparation 2, by heating N1-(4-hydroxybutyl)-2-aminobenzamide (5 grams, 22.8 mmol), obtained in preparation 21, triethylamine (8.06 grams, 78.9 mmol), propionic acid (16 mL) and propionyl chloride (8.4 mL, 89.4 mmol) in xylene (50 mL) at 167° C. for 70 hours. Yield: 2.3 grams, 32%.

¹H NMR (CDCl₃, 300 MHz): δ 1.13 (t, J=7.5 Hz, 3H), 1.41 (t, J=7.4 Hz, 3H), 1.74-1.82 (m, 4H), 2.32 (q, J=7.5 Hz, 2H), 2.85 (q, J=7.4 Hz, 2H), 4.09-4.15 (m, 4H), 7.38-7.44 (m, 1H), 7.60-7.64 (m, 2H), 8.21-8.24 (m, 1H).

### PREPARATION 26

### 2-Ethyl-3-(4-hydroxybutyl)-3,4-dihydro-4-quinazolinone

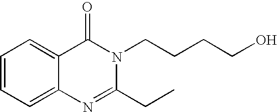

The title compound was prepared by following the same procedure as described in the preparation 3, by using 4-(2-ethyl-4-oxo-3,4-dihydro-3-quinazolinyl)butyl propionate (2.3 grams 7.46 mmol), obtained in preparation 25, and sodium carbonate (1.58 grams, 14.6 mmol) in methanol-water (30 mL, 4:1) at 20 to 40° C. for 4 hours. Yield: 1.8 grams 96.3%.

### PREPARATION 27

### 3-(4-Chlorobutyl)-2-ethyl-3,4-dihydro-4-quinazolinone

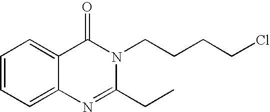

The title compound was prepared by following the same procedure as described in the preparation 4, by using 2-ethyl-3-(4-hydroxybutyl)-3,4-dihydro-4-quinazolinone (1.0 gram, 3.98 mmol), obtained in preparation 26, and thionyl chloride (2.36 grams, 19.6 mmol) in dichloromethane (10 mL) at 20 to 40° C. for 12 to 17 hours.

Yield: 1.01 grams, 93.9%.

### PREPARATION 28

### 4-(4-Oxo-2-propyl-3,4-dihydro-3-quinazolinyl)butyl butyrate

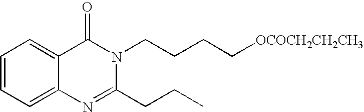

The title compound was prepared following the same procedure as described in the preparation 2, by heating N1-(4-hydroxybutyl)-2-aminobenzamide (5.0 grams, 22.8 mmol), obtained in preparation 21, triethylamine (8.06 grams, 78.9 mmol), butyric acid (10 mL) and butyryl chloride (6.07 grams, 56.6 mmol) in xylene (50 mL) at 167° C. for 48 hours.

Yield: 2.5 grams, 31.6%.

¹H NMR (CDCl₃, 300 MHz): δ 0.93 (t, J=7.4 Hz, 3H), 1.08 (t, J=7.4 Hz, 3H), 1.56-1.69 (m, 2H), 1.76-1.86 (m, 6H), 2.27 (t, J=7.5 Hz, 2H), 2.77 (t, J=7.5 Hz, 2H), 4.08-4.14 (m, 4H), 7.37-7.43 (m, 1H), 7.58-7.72 (m, 2H), 8.22 (dd, J=1.0 & 8.0 Hz, 1H).

### PREPARATION 29

### 3-(4-Hydroxybutyl)-2-propyl-3,4-dihydro-4-quinazolinone

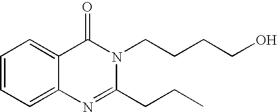

The title compound was prepared by following the same procedure as described in the preparation 3, by using 4-(4-oxo-2-propyl-3,4-dihydro-3-quinazolinyl)butyl butyrate (2.3 grams, 7.46 mmol), obtained in preparation 28, and sodium carbonate (1.58 grams, 14.6 mmol) in methanol-water (30 mL, 23:7) at 20 to 40° C. for 4 hours. Yield: 1.8 grams, 95.6%

¹H NMR (CD₃OD, 300 MHz): δ 1.09 (t, J=7.3 Hz, 3H), 1.65-1.91 (m, 6H), 2.77-2.83 (m, 2H), 3.75 (t, J=6.1 Hz, 2H), 4.14 (t, J=7.8 Hz, 2H), 7.38-7.44 (m, 1H), 7.62 (d, J=7.8 Hz, 1H), 7.67-7.73 (m, 1H), 8.23 (d, J=8.0 Hz, 1H).

### PREPARATION 30

### 3-(4-Chlorobutyl)-2-propyl-3,4-dihydro-4-quinazolinone

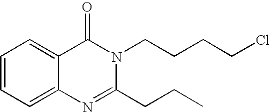

The title compound was prepared by following the same procedure as described in the preparation 4, by using 3-(4-hydroxybutyl)-2-propyl-3,4-dihydro-4-quinazolinone (2 grams, 7.5 mmol), obtained in preparation 29, and thionyl chloride (4.49 grams, 37.35 mmol) in dichloromethane (20 mL) at 20 to 40° C. for 8 hours.

Yield: 2.0 grams, 93.5%.

### PREPARATION 31

### 1-Ethyl-3-propyl-1H-pyrazole carboxylic acid

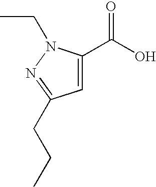

Diethyl sulfate (13.7 grams, 89 mmol) was added to the ethyl 3-propyl-1H-5-pyrazole carboxylate (36.0 grams, 197.8 mmol) and the reaction mixture was heated at 160° C. for 2 hours. The reaction mixture was cooled to ˜90° C. and 5N sodium hydroxide solution (144 mL) was added; and the reaction mixture was stirred at 80-90° C. for 1 hour. The reaction mixture was diluted with cold water, cooled in an ice bath and was acidified to pH 4 with 2N hydrochloric acid resulting in precipitation of the product. The precipitates were filtered, washed with cold water and dried under vacuum to afford the title compound. Yield: 25.2 grams, 70%.

¹H NMR (CDCl₃, 200 MHz): δ 0.98 (t, J=7.3 Hz, 3H), 1.44 (t, J=7.1 Hz, 3H), 1.59-1.81 (m, 2H), 2.63 (t, J=7.6 Hz, 2H), 4.57 (q, J=7.1 Hz, 2H), 6.74 (s, 1H).

### PREPARATION 32

### 1-Ethyl-4-nitro-3-propyl-1H-pyrazole carboxylic acid

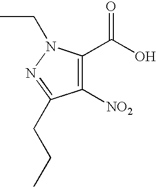

To a 500 mL single neck round bottom flask equipped with CaCl₂ guard containing fuming nitric acid (12.5 mL) heated to 90° C. in a pre heated oil bath, conc. Sulphuric acid (24 mL) was added drop wise and stirred for 10 minutes. 1-Ethyl-3-propyl-1H-pyrazole carboxylic acid (20 grams, 109.9 mmol), obtained in preparation 31, was added portion wise over a period of 20 minutes and stirring was continued for 2 hours. The reaction mixture was allowed to attain 20 to 40° C. It was poured into a beaker containing crushed ice and was stirred well. The separated solids were filtered, washed with cold water and vacuum dried to obtain the title compound as white solid.

Yield: 22.75 grams, 91%.

¹H NMR (CDCl₃, 200 MHz): δ 0.95-1.13 (m, 3H), 1.49 (t, J=7.1 Hz, 3H), 1.66-1.89 (m, 2H), 2.91 (t, J=7.6 Hz, 2H), 4.62 (q, J=7.2 Hz, 2H).

### PREPARATION 33

### 1-Ethyl-4-nitro-3-propyl-1H-5-pyrazole carboxamide

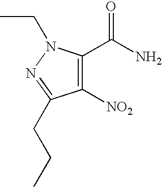

1-Ethyl-4-nitro-3-propyl-1H-pyrazole carboxylic acid (22.75 grams, 100.2 mmol), obtained in preparation 32, was taken in thionyl chloride (45.8 mL, 75.1 mmol) and refluxed for 3.5 hours. Excess thionyl chloride was distilled off, the residue was dissolved in dry acetone (200 mL) and cooled to 0° C. Dry ammonia gas was passed through this solution till pH reached 8-9. The separated solids were filtered and washed with acetone. The solids were dissolved in ethyl acetate, washed the organic layer with water and brine, and dried over sodium sulphate and concentrated. The crude mass was triturated with 10% ethyl acetate in pet ether to afford the title compound as pale yellow solid. Yield: 19.5 grams, 86%.

¹H NMR (CDCl₃, 200 MHz): δ 1.00 (t, J=7.3 Hz, 3H), 1.48 (t, J=7.1 Hz, 3H), 1.62-1.81 (m, 2H), 2.88 (t, J=7.6 Hz, 2H), 4.39 (q, J=7.2 Hz, 2H), 6.22 (brs, 1H, D₂O exchangeable), 7.23 (brs, 1H, D₂O exchangeable).

Mass (CI): m/z 227 (M⁺+1).

### PREPARATION 34

### 4-Amino-1-ethyl-3-propyl-1H-5-pyrazole carboxamide

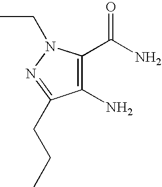

To a solution of 1-ethyl-4-nitro-3-propyl-1H-5-pyrazole carboxamide (19.5 grams, 86.3 mmol), obtained in preparation 33, in ethyl acetate (200 mL) 5% palladium-carbon (13.0 grams) was added and the mixture was hydrogenated under 50 psi hydrogen pressure at 50° C. for 4 hours. The catalysts were filtered through celite bed and the bed was washed with hot ethyl acetate. The filtrate was concentrated. The crude mass was triturated with 10% ethyl acetate in pet ether to give the title compound as brick red solid.

Yield: 14.3 grams, 85%. Melting Point: 81-83° C.

¹H NMR (CDCl₃, 200 MHz): δ 0.97 (t, J=7.4 Hz, 3H), 1.37 (t, J=7.1 Hz, 3H), 1.55-1.79 (m, 2H), 2.56 (t, J=7.5 Hz, 2H), 4.55 (q, J=7.1 Hz, 2H).

### PREPARATION 35

### 1-Ethyl-5-methyl-3-propyl-6,7-dihydro-1H-pyrazolo[4,3-d]pyrimidin-7-one

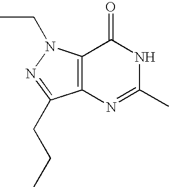

To a cooled solution (0° C.) of 4-amino-1-ethyl-3-propyl-1H-5-pyrazole carboxamide (5.0 grams, 25.5 mmol), obtained in preparation 34, in xylene (27 mL) and propionic acid (27 mL), triethyl amine (7.8 mL, 56.1 mmol) was added drop wise and the reaction mixture was stirred for 15 minutes. Acetyl chloride (2 mL, 28.1 mmol) was added at the same temperature and the reaction mixture was allowed to stir at 20 to 40° C. for 1 hour till the amide formation was over. The reaction mixture was then refluxed for 36 hours. Xylene and propionic acid were removed under reduced pressure. Ice pieces were added to the residue and were stirred well. Separated solids were filtered and washed with water, vacuum dried. The crude mass was purified over silica gel (100-200 mesh) using 10% ethyl acetate in pet ether to afford the title compound as off white fluffy solid. Yield: 4.5 grams, 80%. Melting Point: 211-213° C.

¹H NMR (CDCl₃, 200 MHz): δ 0.99 (t, J=7.4 Hz, 3H), 1.49 (t, J=7.1 Hz, 3H), 1.72-1.90 (m, 2H), 2.52 (s, 3H), 2.87 (t, J=7.5 Hz, 2H), 4.61 (q, J=7.2 Hz, 2H), 10.9 (brs, 1H, D₂O exchangeable).

### PREPARATION 36

### 1,5-Diethyl-3-propyl-6,7-dihydro-1H-pyrazolo[4,3-d]pyrimidin-7-one

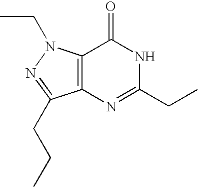

The title compound was prepared by following the same procedure as described in the preparation 35 by using 4-amino-1-ethyl-3-propyl-1H-5-pyrazole carboxamide (5.0 grams, 25.5 mmol), obtained in preparation 34, and propionic acid (27 mL), triethyl amine (7.8 mL, 56.1 mmol) and propionyl chloride (2.5 mL, 28.1 mmol) in xylene (27 mL) at 167° C. for 36 hours. Yield: 4.8 grams, 81%. Melting Point: 137-139° C.

¹H NMR (CDCl₃, 200 MHz): δ 1.00 (t, J=7.2 Hz, 3H), 1.39 (t, J=7.6 Hz, 3H), 1.49 (t, J=7.2 Hz, 3H), 1.75-1.92 (m, 2H), 2.77 (q, J=7.6 Hz, 2H), 2.88 (t, J=7.6 Hz, 2H), 4.61 (q, J=7.2 Hz, 2H), 10.79 (brs, 1H, D₂O exchangeable).

Mass (CI): m/z 235 (M⁺+1).

### PREPARATION 37

### 6-(3-Bromopropyl)-1,5-diethyl-3-propyl-6,7-dihydro-1H-pyrazolo[4,3-d]pyrimidin-7-one

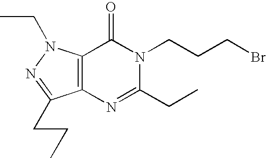

To a solution of 1,5-diethyl-3-propyl-6,7-dihydro-1H-pyrazolo[4,3-d]pyrimidin-7-one (0.5 grams, 2.136 mmol), obtained in preparation 36, in dry dimethylformamide (5.0 mL) potassium carbonate (885 mg, 6.41 mmol) was added and was stirred for 30 minutes. 1,3-Dibromo propane (1.1 mL, 10.68 mmol) was then added and the mixture was stirred at 20 to 40° C. for 24 hours. The reaction mixture was diluted with water and extracted with ethyl acetate twice. The combined organic layers were washed with water, brine, dried over anhydrous sodium sulphate and concentrated. The crude mass was chromatographed on silica gel (100-200 mesh) using 15% ethyl acetate in pet ether to afford the title compound as yellow colored liquid. Yield: 0.332 grams, 44%.

¹H NMR (CDCl₃, 200 MHz): δ 0.99 (t, J=7.3 Hz, 3H), 1.38 (t, J=7.2 Hz, 3H), 1.46 (t, J=7.0 Hz, 3H), 1.70-1.93 (m, 2H), 2.14-2.38 (m, 2H), 2.72-2.98 (m, 4H), 3.52 (t, J=6.2 Hz, 2H), 4.23 (t, J=7.7 Hz, 2H), 4.60 (q, J=7.2 Hz, 2H).

### PREPARATION 38

### 6-(3-Bromopropyl)-1-ethyl-5-methyl-3-propyl-6,7-dihydro-1H-pyrazolo[4,3-d]-pyrimidin-7-one

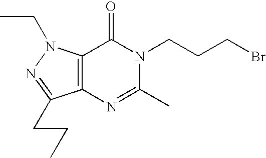

The title compound was prepared as white solid following the same procedure as described in the preparation 37 by using 1-ethyl-5-methyl-3-propyl-6,7-dihydro-1H-pyrazolo[4,3-d]pyrimidin-7-one (0.5 grams, 2.27 mmol), obtained in preparation 35, potassium carbonate (0.941 grams, 6.8 mmol) and 1,3-dibromo propane (1.16 mL, 11.36 mmol) in dimethylformamide (5 mL) at 20 to 40° C. for 24 hours. Yield: 0.418 grams, 54%. Melting Point: 60-62° C.

¹H NMR (CDCl₃, 200 MHz): δ 0.99 (t, J=7.3 Hz, 3H), 1.46 (t, J=7.2 Hz, 3H), 1.66-1.93 (m, 2H), 2.20-2.40 (m, 2H), 2.65 (s, 3H), 2.85 (t, J=7.6 Hz, 2H), 3.5 (t, J=6.2 Hz, 2H), 4.23 (t, J=7.7 Hz, 2H), 4.60 (q, J=7.1 Hz, 2H).

### PREPARATION 39

### Ethyl-1-methyl-7-oxo-3-propyl-6,7-dihydro-1H-pyrazolo[4,3-d]pyrimidine-5-carboxylate

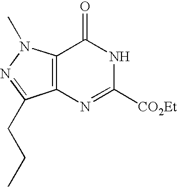

To a stirred solution of 4-amino-1-methyl-3-propyl-1H-5-pyrazole carboxamide (8.0 grams, 44 mmol) in xylene and propionic acid mixture (80 mL, 1:1) triethyl amine (13.6 mL, 96.7 mmol) was added at 0° C. After 15 minutes, ethyl oxalylchloride (5.9 mL, 52.74 mmol) was added at 0° C. The stirring was continued for a period of 1.5 hours. When the amide formation was over the reaction mixture was set for reflux at 160° C. and was continued for 48 hours. Xylene and propionic acid were distilled off from the reaction mixture, diluted with ethyl acetate and washed with water followed by brine, dried (sodium sulphate) and concentrated. The residue was purified through a column of 100-200 mesh silica gel using 30% ethyl acetate in pet ether to afford the title compound.

Yield: 2 grams, 25%.

¹H NMR (CDCl₃, 200 MHz): δ 1.00 (t, J=7.4 Hz, 3H), 1.50 (t, J=7.4 Hz, 3H), 1.81 (q, J=7.5 Hz, 2H), 2.89 (t, J=7.6 Hz, 2H), 4.29 (s, 3H), 4.57 (q, J=7.5 Hz, 2H), 9.00 (brs, 1H, D₂O exchangeable).

### PREPARATION 40

### 1-Methyl-3-propyl-6,7-dihydro-1H-pyrazolo[4,3-d]pyrimidin-7-one

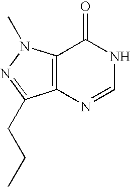

To a solution of ethyl-1-methyl-7-oxo-3-propyl-6,7-dihydro-1H-pyrazolo[4,3-d]pyrimidine-5-carboxylate (7.52 grams, 28.5 mmol) in methanol (70 mL) a solution of lithium hydroxide (1.79 grams, 42.75 mmol) in water (20 mL) was added and the reaction mixture was stirred at 20 to 40° C. for 12 hours. The solvents were removed under reduced pressure and the residue was dissolved in water. The aqueous layer was extracted with ethyl acetate and the organic layer was discarded. The aqueous layer was acidified up to pH 2 at 0° C., with 2N hydrochloric acid and was extracted with ethyl acetate. The organic layer was washed with brine, dried over sodium sulfate and concentrated to afford the title compound. Yield: 5.0 grams, 91.5%. Melting Point: 199-200° C.

¹H NMR (CDCl₃, 200 MHz): δ 1.00 (t, J=7.4 Hz, 3H), 1.81 (q, J=7.5 Hz, 2H), 2.89 (t, J=7.6 Hz, 2H), 4.29 (s, 3H), 7.86 (s, 1H).

### PREPARATION 41

### 6-(2-Bromoethyl)-1-methyl-3-propyl-6,7-dihydro-1H-pyrazolo[4,3-d]pyrimidin-7-one

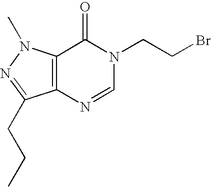

The title compound was obtained as pale yellow liquid following the same procedure as described in the preparation 37 by using 1-methyl-3-propyl-6,7-dihydro-1H-pyrazolo-[4,3-d]pyrimidin-7-one (3 grams, 15.54 mmol), obtained in preparation 40, potassium carbonate (6.4 grams, 46.6 mmol) and 1,2-dibromo ethane (1.16 mL, 11.36 mmol) in dimethylformamide (30 mL) at 20 to 40° C. for 36 hours. Yield: 3.75 grams, 81%.

¹H NMR (CDCl₃, 200 MHz): δ 1.01 (t, J=7.4 Hz, 3H), 1.81 (q, J=7.4 Hz, 2H), 2.87 (t, J=7.7 Hz, 2H), 3.74 (t, J=5.8 Hz, 2H), 4.26 (s, 3H), 4.36 (t, J=5.9 Hz, 2H), 7.83 (s, 1H). Mass (CI): m/z 299 (M⁺).

### PREPARATION 42

### 6-(3-Bromopropyl)-1-methyl-3-propyl-6,7-dihydro-1H-pyrazolo[4,3-d]pyrimidin-7-one

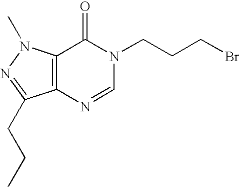

The title compound was obtained as pale yellow liquid following the same procedure as described in the preparation 37 by using 1-methyl-3-propyl-6,7-dihydro-1H-pyrazolo[4,3-d]pyrimidin-7-one (200 mg, 1.03 mmol), obtained in preparation 40, potassium carbonate (429 mg, 3.1 mmol) and 1,3-dibromo propane (627.5 mg, 3.11 mmol) in dimethylformamide (6 mL) at 20 to 40° C. for 16 hours. Yield: 150 mg, 46%.

¹H NMR (CDCl₃, 200 MHz): δ 1.00 (t, J=7.3 Hz, 3H), 1.70-1.92 (m, 2H), 2.25-2.45 (m, 2H), 2.86 (t, J=7.6 Hz, 2H), 3.44 (t, J=6.2 Hz, 2H), 4.18 (t, J=6.6 Hz, 2H), 4.26 (s, 3H), 7.85 (s, 1H). Mass (CI): m/z 313 (M⁺+1).

### PREPARATION 43

### 3-(2-Methyl-4-oxo-3,4-dihydro-3-quinazolinyl)propyl methanesulfonate

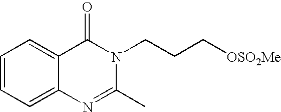

To a cooled (0° C.) solution of 3-(3-hydroxypropyl)-2-methyl-4-Oxo-3,4-dihydro-3-quinazolinone (4.0 grams, 18.35 mmol), obtained in preparation 13, in dichloromethane (80 mL) triethylamine (3.71 grams, 36.7 mmol) was added drop wise and was stirred for 15 minutes. Methanesulfonylchloride (4.2 grams, 36.7 mmol) was then added to the reaction mixture and was stirred 12 to 17 hours at 20 to 40° C. The reaction mixture was washed successively with water and brine, dried (sodium sulphate) and concentrated to afford the title compound, which was used in the next step without purification. Yield: 5.42 grams. Melting Point: 148-150° C.

¹H NMR (CDCl₃, 200 MHz): δ 2.17-2.3.1 (m, 2H), 2.72 (s, 3H), 2.75 (s, 3H), 4.27 (t, J=7.3 Hz, 2H), 4.39 (t, J=5.9 Hz, 2H), 7.47 (t, J=7.4 Hz, 1H), 7.63-7.81 (m, 2H), 8.23 (d, J=7.3 Hz, 1H). Mass (CI): m/z 297 (M⁺+1).

### PREPARATION 44

### 1-Ethyl-6-(2-hydroxyethyl)-5-methyl-3-propyl-6,7-dihydro-1H-pyrazolo[4,3-d]-pyrimidin-7-one

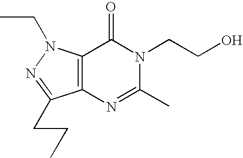

The title compound was prepared as white solid following the same procedure as described in the preparation 37 by heating 1-ethyl-5-methyl-3-propyl-6,7-dihydro-1H-pyrazolo[4,3-d]pyrimidin-7-one (2 grams, 9.09 mmol), obtained in preparation 35, potassium carbonate (3.8 grams, 27.27 mmol) and 2-bromopropanol (3.2 mL, 45-45 mmol) in dimethylformamide (20 mL) at 60° C. for 48 hours. Yield: 1.3 grams, 54%. Melting Point: 118-120° C.

¹H NMR (CDCl₃, 200 MHz): δ 0.99 (t, J=7.3 Hz, 3H), 1.45 (t, J=7.2 Hz, 3H), 1.68-1.90 (m, 2H), 2.66 (s, 3H), 2.84 (t, J=7.7 Hz, 2H), 3.91-4.08 (m, 2H), 4.30 (t, J=5.2 Hz, 2H), 4.58 (q, J=7.2 Hz, 2H).

### PREPARATION 45

### 6-(2-Chloroethyl)-1-ethyl-5-methyl-3-propyl-6,7-dihydro-1H-pyrazolo[4,3-d]-pyrimidin-7-one

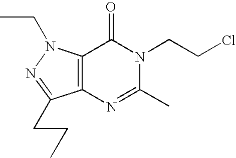

The title compound was prepared as a white solid following the same procedure as described in the preparation 43 by using 1-ethyl-6-(2-hydroxyethyl)-5-methyl-3-propyl-6,7-dihydro-1H-pyrazolo[4,3-d]-pyrimidin-7-one (1.2 grams, 4.5 mmol), obtained in preparation 44, triethylamine (1.9 mL, 13.6 mmol) and methanesulfonylchloride (0.71 mL, 9.1 mmol) in dichloromethane (15 mL) at 20 to 40° C. for 36 hours. Yield: 0.88 grams, 69%. Melting Point: 81-83° C.

¹H NMR (CDCl₃, 200 MHz): δ 1.0 (t, J=7.3 Hz, 3H), 1.47 (t, J=7.2 Hz, 3H), 1.70-1.94 (m, 2H), 2.69 (s, 3H), 2.85 (t, J=7.7 Hz, 2H), 3.86 (t, J=6.3 Hz, 2H), 4.41 (t, J=6.3 Hz, 2H), 4.59 (q, J=7.2 Hz, 2H).

### PREPARATION 46

### 1,5-Diethyl-6-(2-hydroxyethyl)-3-propyl-6,7-dihydro-1H-pyrazolo[4,3-d]pyrimidin-7-one

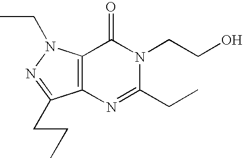

The title compound was prepared as pale yellow liquid following the same procedure as described in the preparation 37 by heating 1,5-diethyl-3-propyl-6,7-dihydro-1H-pyrazolo[4,3-d]pyrimidin-7-one (2 grams, 8.54 mmol), obtained in preparation 36, potassium carbonate (3.5 grams, 25.6 mmol) and 2-bromopropanol (3.0 mL, 42.7 mmol) in dimethylformamide (20 mL) at 60° C. for 48 hours. Yield: 2.1 grams, 89%.

¹H NMR (CDCl₃, 200 MHz): δ 0.98 (t, J=7.3 Hz, 3H), 1.26 (t, J=7.2 Hz, 3H), 1.35 (t, J=7.3 Hz, 3H), 1.70-1.91 (m, 2H), 2.79-3.00 (m, 4H), 4.10 (t, J=7.2 Hz, 2H), 4.30 (t, J=5.3 Hz, 2H), 4.57 (q, J=7.3 Hz, 2H). Mass (CI): m/z 279 (M⁺+1).

### PREPARATION 47

### 6-(2-Chloroethyl)-1,5-diethyl-3-propyl-6,7-dihydro-1H-pyrazolo[4,3-d]pyrimidin-7-one

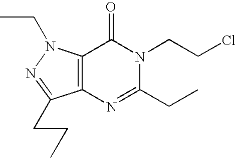

The title compound was prepared as a white solid following the same procedure as described in the preparation 43 by using 1,5-diethyl-6-(2-hydroxyethyl)-3-propyl-6,7-dihydro-1H-pyrazolo[4,3-d]pyrimidin-7-one (2.1 grams, 7.55 mmol), obtained in preparation 46, triethylamine (3.15 grams, 22.66 mmol) and methanesulfonylchloride (1.17 mL, 15.1 mmol) in dichloromethane (20 mL) at 20 to 40° C. for 36 hours.

Yield: 1.08 grams, 48%. Melting Point: 61-63° C.

¹H NMR (CDCl₃, 200 MHz): δ 0.99 (t, J=7.3 Hz, 3H), 1.36 (t, J=7.3 Hz, 3H), 1.46 (t, J=7.2 Hz, 3H), 1.70-1.94 (m, 2H), 2.89-2.99 (m, 4H), 3.82 (t, J=6.6 Hz, 2H), 4.41 (t, J=6.6 Hz, 2H), 4.59 (q, J=7.1 Hz, 2H).

### PREPARATION 48

### N1-{4-[2-(5-ethyl-1-methyl-7-oxo-3-propyl-6,7-dihydro-1H-pyrazolo[4,3-d]-pyrimidin-6-yl)ethoxy]phenyl}acetamide

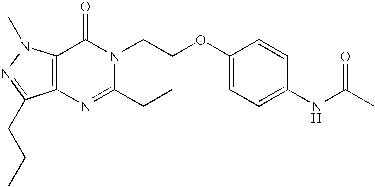

A mixture of 4-acetamido phenol (0.78 grams, 5.16 mmol) and potassium carbonate (2.13 grams, 15.5 mmol) in dry dimethylformamide (10 mL) was stirred at 20 to 40° C. for 45 minutes. 6-(2-chloroethyl)-5-ethyl-1-methyl-3-propyl-6,7-dihydro-1H-pyrazolo[4,3-d]pyrimidin-7-one (1.75 grams, 6.19 mmol) in dimethylformamide (8 mL) was added slowly drop wise and was stirred at 60° C. for 15 hours. Reaction mixture was cooled to 20 to 40° C., 200 mL of ethyl acetate was added, washed with water. Aqueous layer was extracted with ethyl acetate twice. Combined organic extracts were washed with water, brine, dried over anhydrous sodium sulfate and evaporated under reduced pressure. Crude product was purified on 100-200 mesh silica gel using 30% ethyl acetate in pet ether to afford the title compound as white solid. Yield: 1.83 grams, 89%. Melting Point: 100-104° C.

¹H NMR (CDCl₃, 200 MHz): δ 0.99 (t, J=7.3 Hz, 3H), 1.38 (t, J=7.3 Hz, 3H), 1.74-1.92 (m, 2H), 2.13 (s, 3H), 2.85 (t, J=7.6 Hz, 2H), 3.03 (q, J=7.3 Hz, 2H), 4.20-4.32 (m, 5H), 4.49 (t, J=5.1 Hz, 2H), 6.79 (d, J=8.8 Hz, 2H), 7.35 (d, J=9.3 Hz, 2H).

### PREPARATION 49

### 6-[2-(4-Aminophenoxy)ethyl]-5-ethyl-1-methyl-3-propyl-6,7-dihydro-1H-pyrazolo-[4,3-d]pyrimidin-7-one

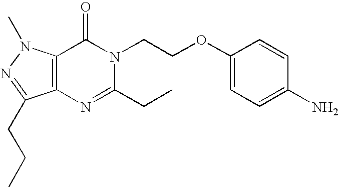

N1-{4-[2-(5-Ethyl-1-methyl-7-oxo-3-propyl-6,7-dihydro-1H-pyrazolo[4,3-d]-pyrimidin-6-yl)ethoxy]phenyl}acetamide (150 mg, 0.378 mmol), obtained in preparation 48, in 6N hydrochloric acid (2 mL) was stirred at 55-60° C. for 18 hours. Reaction mixture was cooled to 0° C. and basified with saturated sodium bicarbonate solution up to pH 8-9 to afford off white solid, which was filtered and dried.

Yield: 125 mg, 93%. Melting Point 138-142° C.

¹H NMR (CDCl₃, 200 MHz): δ 1.00 (t, J=7.3 Hz, 3H), 1.37 (t, J=7.3 Hz, 3H), 1.72-1.90 (m, 2H), 2.86 (t, J=7.6 Hz, 2H), 3.03 (q, J=7.3 Hz, 2H), 3.43 (brs, 1H, D₂O exchangeable), 4.18-4.26 (m, 5H), 4.47 (t, J=5.4 Hz, 2H), 6.6 (d, J=8.8 Hz, 2H), 6.68 (d, J=9.3 Hz, 2H). Mass (EI): m/z 356 (M⁺+1).

### PREPARATION 50

### 2-(4-Hydroxy-phenoxy)-2-methyl-propionic acid ethyl ester

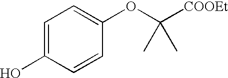

Hydroquinone (20 grams, 181.8 mmol) and ethyl 2-bromo isobutyrate (13 mL, 90.9 mmol) were refluxed in ethanol (200 mL) with potassium hydroxide (5.1 grams, 90.9 mmol) for 24 hours. Another portion of ethyl 2-bromo isobutyrate (13 mL) was added and the reflux was continued for 48 hours. Ethanol was removed, the residue was taken in ethyl acetate, washed with water and brine, dried anhydrous sodium sulfate and evaporated to dryness. Column purification with 30% ethyl acetate in pet ether to obtain pure titled compound. Yield: 9.06 grams, 22%.

¹HNMR (CDCl₃, 200 MHz): δ 6.79 and 6.69 (2d, J=9.1 Hz, 4H), 4.24 (q, J=7.2 Hz, 2H), 1.53 (s, 6H), 1.28 (t, J=7.3 Hz, 3H). MS (CI): m/z 225 (M⁺+1).

IR (cm⁻¹) (Neat): 2986, 1720, 1512.

### PREPARATION 51

### 2-(4-Amino-phenoxy)-2-methyl-propionic acid ethyl ester

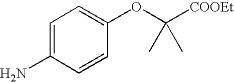

To a stirred solution of 4-nitrophenol (15 grams, 107.8 mmol) in dry dimethylformamide (150 mL) potassium carbonate was added and stirred at 20 to 40° C. for 30 minutes. Ethyl 4-bromoisobutyrate was added to it drop wise and stirred for further 18 hours. Water was added to the reaction mixture and extracted with ethyl acetate. Combined organic layers were washed with water and brine, dried over sodium sulphate and evaporated to dryness. The residue was purified 10% ethyl acetate in pet ether to obtain ethyl 2-methyl-2-(4-nitrophenoxy)propanoate.

To a solution of ethyl 2-methyl-2-(4-nitrophenoxy)propanoate (4 grams, 15.8 mmol) in ethyl acetate (40 mL) 10% palladium-carbon (2 grams) was added hydrogenated at 20 to 40° C. for 16 hours. Solids were filtered through a celite bed, washed the bed with ethyl acetate. Removal of the solvents gave the titled compound.

Yield: 3.46 grams, 98%.

¹H NMR (CDCl₃, 200 MHz): δ 6.74 (d, J=8.6 Hz, 2H), 6.56 (d, J=8.9 Hz, 2H), 4.23 (q, J=7.2 Hz, 2H), 1.28 (t, J=7.1 Hz, 3H). Mass (ES): m/z 224 (M⁺+1).

IR (cm⁻¹) (KBr): 3371, 2939, 1731, 1627, 1510.

### PREPARATION 52

### 2-(3-Hydroxy-phenoxy)-2-methyl-propionic acid ethyl ester

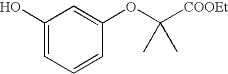

To a cold solution of resorcinol (5 grams, 45.4 mmol) in dimethylformamide (50 mL) sodium hydride (2.7 grams, 67.65 mmol, 60% oil coated) was added and stirred for 30 minutes. Ethyl 2-bromo isobutyrate (7.9 mL, 54 mmol) was added dropwise and stirred at 20 to 40° C. for 48 hours. The reaction mixture was diluted with ethyl acetate, washed with water and brine, dried over sodiumsulphate and evaporated to dryness. Column purification with 20% ethyl acetate in pet ether to yield pure title compound.

Yield: 4.09 g, 40%.

¹H NMR (CDCl₃, 200 MHz): δ 7.06 (t, J=8.4 Hz, 4H), 650-6.34 (m, 3H), 4.23 (q, J=7.1 Hz, 2H), 1.59 (s, 6H), 1.24 (t, J=7.2 Hz, 3H).

Mass (CI): m/z 225 (M⁺+1).

IR (cm⁻¹): 2989, 1731, 1596, 1486.

### PREPARATION 53

### Ethyl 1-(4-hydroxyphenoxy)-1-cyclobutanecarboxylate

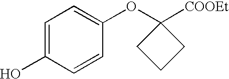

To a cold solution of hydroquinone (6 grams, 54.49 mmol) in dimethylformamide (50 mL) sodium hydride (3.24 grams, 81.2 mmol, 60% oil coated) was added and stirred for 30 min. Ethyl 1-bromo-1-cyclobutanecarboxylate (13.4 grams, 64.8 mmol) in dimethylformamide (10 mL) was added dropwise and stirred at 20 to 40° C. for 8 days. The reaction mixture was diluted with ethyl acetate, washed with water and brine, dried over sodiumsulphate and evaporated to dryness. Column purification with 10% ethyl acetate in pet ether to give pure title compound. Yield: 1.45 g, 11.3%. Melting Point: 118-120° C.

¹H NMR (CDCl₃, 400 MHz): δ 6.68 (dd, J=6.6 and 2.4 Hz, 2H), 6.56 (dd, J=6.6 and 2.4 Hz, 2H), 4.19 (q, J=7.1 Hz, 2H), 2.73-2.66 (m, 2H), 2.46-2.38 (m, 2H), 2.04-1.93 (m, 2H), 1.18 (t, J=7.1 Hz, 3H). Mass (CI): m/z 237 (M⁺+1).

IR (cm⁻¹): 2943, 1719, 1512.

### PREPARATION 54

### 2-(3-Hydroxy-phenoxy)-2-methyl-butyric acid ethyl ester

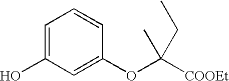

To a cold solution of resorcinol (5 grams, 45.4 mmol) in dimethylformamide (50 mL) sodium hydride (2.7 grams, 67.65 mmol, 60% oil coated) was added and stirred for 30 minutes. Ethyl 2-bromo isovalarate (11.29 g, 54 mmol) was added dropwise and stirred at 20 to 40° C. for 48 hours. The reaction mixture was diluted with ethyl acetate, washed with water and brine, dried over sodiumsulphate and evaporated to dryness. Column purification with 20% ethyl acetate in pet ether to give the pure title compound

Yield: 1.55 g, 14.3%.

¹H NMR (CDCl₃, 200 MHz): δ 7.07 (t, J=7.9 Hz, 1H), 6.48-6.39 (m, 3H), 4.24 (s, 3H), 2.05-1.83 (m, 2H), 1.26 (t, J=7.0 Hz, 3H), 0.98 (t, J=7.6 Hz, 3H). Mass (CI): m/z 239 (M⁺+1).

IR (cm⁻¹): 2942, 1731, 1596.

### PREPARATION 55

### 3-Benzyloxy phenol

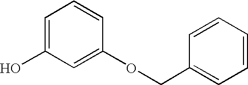

Benzene-1,3-diol (10.0 grams, 90.9 mmol), dissolved in acetone (50 mL), was cooled to 5-10° C., and added with anhydrous potassium carbonate (18.8 grams, 136.3 mol) under smooth stirring. Benzyl bromide (10.88 grams, 63.6 mmol) was slowly introduced to the reaction mass at this temperature and cooling bath was removed after ten minutes. The reaction mixture was refluxed for 12 to 17 hours. After bringing to 20 to 40° C., the solid potassium carbonate was filtered out. The filtrate was concentrated and poured over ice-water, acidified with 6 N hydrochloric acid and extracted with ethyl acetate (3×25 mL). The combined organic layer was washed with water, dried with anhydrous sodiumsulphate and evaporated to get brown colored gummy mass. The dibenzyloxy bye-product was discarded by column chromatographic purification using 230-400 mesh silica-gel and mixture of ethyl acetate and petroleum ether (10:90) to yield pure a light brown gummy mass title compound. Yield: 13.5 g, 68%

¹H NMR (CDCl₃, 200 MHz): δ 7.50-7.30 (m, 5H), 7.12 (t, J=8.2 Hz, 1H), 6.57 (dd, J=1.8 & 6.2 Hz, 1H), 6.50-6.40 (m, 2H), 5.02 (s, 2H), 4.86 (bs, 1H, D₂O exchangeable). Mass (CI): m/z 201 (M⁺+1). IR (cm⁻¹) (KBr): 3406, 3032, 1595, 1490, 1454.

### PREPARATION 56

### 2-(3-Benzyloxy-phenoxy)-2-methyl butyric acid

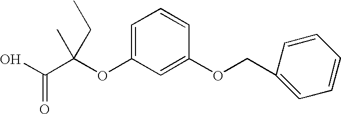

3-Benzyloxy phenol (13.0 grams, 65.0 mmol), obtained in preparation 55, dissolved in toluene (100 mL), was slowly added with powdered sodium hydroxide (20.8 grams, 520.0 mmol) at 20 to 40° C. under smooth stirring. After stirring this reaction mass for 30 minutes, 2-butanone (23.1 grams, 325.0 mmol) was slowly introduced at 20 to 40° C. to the reaction mass. Triethyl benzylammonium chloride (2.0 grams, 6.5 mmol) was subsequently added to the reaction mixture and whole mass was further stirred at 20 to 40° C. for 30 minutes. Bromoform (65.0 grams, 260.0 mmol) was drop-wise added to the reaction mixture at 20 to 40° C. Initially, the temperature increased to 60-65° C. which was controlled by introducing cold water bath. The reaction mixture was stirred at 20 to 40° C. for 4 hours. The reaction mixture was poured over ice-water, stirred and extracted with toluene (3×50 mL). The aqueous layer was acidified with 6N hydrochloric acid and extracted with ethyl acetate (3×50 mL). The combined organic layer was washed with water, dried over anhydrous sodiumsulphate and evaporated to get brown colored gummy mass which was purified by column chromatography using 230-400 mesh silica-gel and mixture of ethyl acetate and petroleum ether (80:20 ratio) to afford a low melting brown solid of the title compound. Yield: 6.82 g, 35%.

¹H NMR (CDCl₃, 200 MHz): δ 7.44-7.32 (m, 5H), 7.18 (t, J=8.2 Hz, 1H), 6.73 (dd, J=1.6 & 6.0 Hz, 1H), 6.60-6.58 (m, 2H), 5.03 (s, 2H), 2.05-1.95 (m, 2H), 1.46 (s, 3H), 1.02 (t, J=7.6 Hz, 3H). Mass (CI): m/z 301 (M⁺+1).

IR (cm⁻¹) (Neat): 3425, 3033, 2941, 1715, 1599, 1486, 1455, 1380.

### PREPARATION 57

### R-(+)-phenylethyl amine salt of (RS)-2-(3-benzyloxy-phenoxy)-2-methyl butyric acid

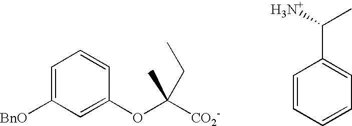

2-(3-Benzyloxy-phenoxy)-2-methyl butyric acid (6.5 grams, 21.6 mmol), obtained in preparation 56, dissolved in 20% ethylacetate-petroleum ether (50 mL), was added with R-(+)-phenylethyl amine (2.62 grams, 21.6 mmol) at 20 to 40° C., and the reaction mixture was refluxed for 1 hour. The reaction mixture was brought to 20 to 40° C. and agitated there for 1 hour. The solid separated was collected by filtration and washed with minimum volume of 20% ethylacetate-petroleum ether to get the mixture of diastereomeric salts. This solid was subjected to twenty six consecutive fractional crystallizations with a mixture of ethylacetate and petroleum ether (1:1, 3.0 vol. each time). No clear solution was observed during heating. The slurry was refluxed for 30 minutes during each crystallization and was always filtered after stirring at 20 to 40° C. for 30 minutes to finally afford the desired title compound Yield: 0.3 g, ˜4.0%. Melting Point: 185-187° C.

¹H NMR (DMSO-d₆, 400 MHz): δ 7.54-7.20 (m, 10H), 7.16 (t, J=8.6 Hz, 1H), 6.75 (dd, J=1.6 & 6.0 Hz, 1H), 6.62-6.55 (m, 2H), 5.05 (s, 2H), 4.15 (q, J=6.8 Hz, 1H), 3.20 (bs, 2H), 1.84-1.74 (m, 2H), 1.38 (d, J=6.8 Hz, 3H), 1.31 (s, 3H), 0.84 (t, J=7.6 Hz, 3H).

Mass (CI): m/z 301 (M⁺+1). IR (cm⁻¹) (KBr): 3512, 2956, 1570, 1476.

### PREPARATION 58

### R-(+)-2-(3-Benzyloxy-phenoxy)-2-methyl butyric acid methyl ester

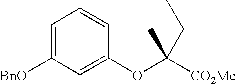

R-(+)-phenylethyl amine salt of (RS)-2-(3-benzyloxy-phenoxy)-2-methyl butyric acid (0.3 grams, 0.71 mmol), obtained in preparation 57, was dissolved in methanol (10 mL) and concentrated sulfuric acid (0.2 mL) was drop-wise added under stirring at 20 to 40° C. The reaction mixture was refluxed for 2-3 hours. Then the reaction mixture was cooled to 20 to 40° C., poured over ice-water and extracted with ethyl acetate (3×10 mL). The combined organic layer was washed with water, dried over anhyd sodium sulphate and evaporated to afford a light colored gummy mass of the titled compound Yield: 0.20 g, 90%.

¹H NMR (CDCl₃, 200 MHz): δ 7.45-7.34 (m, 5H), 7.22 (t, J=8.8 Hz, 1H), 6.77 (dd, J=1.6 & 6.0 Hz, 1H), 6.60-6.50 (m, 2H), 5.03 (s, 2H), 3.75 (s, 3H), 2.00-1.85 (m, 2H), 1.44 (s, 3H), 0.98 (t, J=7.0 Hz, 3H). Mass (CI): m/z 315 (M⁺+1).

IR (cm⁻¹) (Neat): 2945, 1737, 1582, 1487.

### PREPARATION 59

### PREPARATION of R-(+)-2-(3-hydroxyphenoxy)-2-methyl butyric acid methyl ester

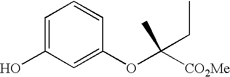

R-(+)-2-(3-benzyloxy-phenoxy)-2-methyl butyric acid methyl ester (0.20 grams, 0.63 mmol), obtained in preparation 58, was dissolved in ethanol (10 mL) and moist palladium carbon (0.10 gram, 50% w/w) was added. The reaction mass was hydrogenated at 40 psi pressure of hydrogen at 20 to 40° C. for 3 hours. The reaction mixture was filtered through 60-120 mesh silica gel column. After washing the column with methanol (3×10 mL), the combined reaction mixture was completely evaporated to get the title compound. Yield: 140 mg, 98%.

¹H NMR (CDCl₃, 200 MHz): δ 7.11 (t, J=8.0 Hz, 1H), 6.48-6.40 (m, 2H), 6.36-6.34 (m, 1H), 4.86 (bs, 1H, D₂O exchangeable), 3.77 (s, 3H), 2.25-1.85 (m, 2H), 1.51 (s, 3H), 0.96 (t, J=7.6 Hz, 3H). Mass (CI): m/z 224 (M⁺+1).

IR (cm⁻¹) (KBr): 3416, 2950, 1732, 1594, 1485.

### PREPARATION 60

### 3-Benzyloxy-4-chlorophenol

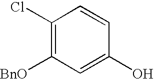

4-Chloro-benzene-1,3-diol (300.0 grams, 2.10 mol), dissolved in acetone (2.5 Liters), was cooled to 5-10° C., and added with anhydrous potassium carbonate (571.3 grams, 4.14 mol) under smooth stirring. Benzyl bromide (284.0 g, 1.70 mol) was slowly introduced to the reaction mass at this temperature and cooling bath was removed after ten minutes. The reaction mixture was refluxed for 12 to 17 hours. After bringing to 20 to 40° C., the solid potassium carbonate was filtered out. The filtrate was concentrated and poured over ice-water, acidified with 6 N hydrochloric acid and extracted with ethyl acetate (3×500 mL). The combined organic layer was washed with water, dried with anhydrous sodiumsulphate and evaporated to get brown colored gummy mass. The dibenzyloxy bye-product was discarded by column chromatographic purification using 230-400 mesh silica-gel and mixture of ethyl acetate and petroleum ether (10:90) to yield pure title compound. Yield: 135 g, 38%.

¹H NMR (CDCl₃, 200 MHz): δ 9.64 (s, 1H, D₂O exchangeable), 7.46-7.23 (m, 5H), 7.17 (d, J=8.4 Hz, 1H), 6.60 (d, J=2.4 Hz, 1H), 6.37 (dd, J=2.4 & 6.0 Hz, 1H), 5.13 (s, 2H). Mass (CI): m/z 235 (M⁺+1). IR (cm⁻¹) (KBr): 3417, 1591, 1529, 1492, 1449, 1174.

### PREPARATION 61

### RS-2-(3-Benzyloxy-4-chlorophenoxy)-2-methyl butyric acid

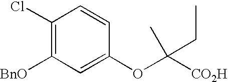

3-Benzyloxy-4-chlorophenol (150.0 grams, 0.64 mol), obtained in preparation 60, dissolved in toluene (1.5 Liters), was slowly added with powdered sodium hydroxide (204.0 grams, 5.1 mol) at 20 to 40° C. under smooth stirring. After stirring this reaction mass for 30 minutes, 2-butanone (460.0 grams, 6.38 mol) was slowly introduced at 20 to 40° C. to the reaction mass. Triethyl benzylammonium chloride (15.0 grams, 0.065 mol) was subsequently added to the reaction mixture and whole mass was further stirred at 20 to 40° C. for 30 minutes. Bromoform (646.0 grams, 2.55 mol) was drop-wise added to the reaction mixture at 20 to 40° C. Initially, the temperature increased to 60 to 65° C. which was controlled by introducing cold water bath. The reaction mixture was stirred at 20 to 40° C. for 4 hours. The reaction mixture was poured over ice-water, stirred and extracted with toluene (3×500 mL). The aqueous layer was acidified with 6 N hydrochloric acid and extracted with ethyl acetate (3×700 mL). The combined organic layer was washed with water, dried over anhydrous sodiumsulphate and evaporated to get brown colored gummy mass which was purified by column chromatography using 230-400 mesh silica-gel and mixture of ethyl acetate and petroleum ether (80:20) to afford title compound.

Yield: 60.0 g, 28%. Melting Point: 51-52° C.

¹H NMR (CDCl₃, 400 MHz): δ 7.43-7.40 (m, 2H), 7.38-7.36 (m, 2H), 7.35-7.34 (m, 1H), 7.24 (dd, J=2.8 & 8.8 Hz, 1H), 6.58 (d, J=2.4 Hz, 1H), 6.47 (dd, J=2.4 & 6.0 Hz, 1H), 5.11 (s, 2H), 2.20-1.90 (m, 1H), 1.85-1.80 (m, 1H), 1.40 (s, 3H), 0.99 (t, J=7.6 Hz, 3H).

Mass (CI): m/z 335 (M⁺+1). IR (cm⁻¹) (KBr): 3515, 2985, 1702, 1581, 1408.

### PREPARATION 62

### R-(+)-phenylethyl amine salt of (RS)-2-(3-benzyloxy-4-chlorophenoxy)-2-methyl butyric acid

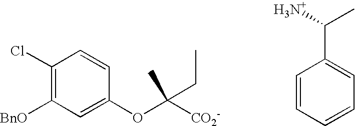

(RS)-2-(3-benzyloxy-4-chlorophenoxy)-2-methyl butyric acid (15.0 g, 44.84 mol), obtained in preparation 61, dissolved in 20% ethylacetate-petroleum ether (150 mL), was added with R-(+)-phenylethyl amine (5.42 g, 44.84 mol) at 20 to 40° C., and the reaction mixture was refluxed for 1 hour. The reaction mixture was brought to 20 to 40° C. and agitated there for 1 hour. The solid separated was collected by filtration and washed with minimum volume of 20% ethylacetate-petroleum ether to get the mixture of diastereomeric salts. This solid was subjected to six consecutive fractional crystallizations with a mixture of ethylacetate and petroleum ether (1:1, 2.5 vol. each time). No clear solution was observed during heating. The slurry was refluxed for 30 minutes during each crystallization and was always filtered after stirring at 20 to 40° C. for 30 minutes to finally afford the desired single title compound.

Yield: 4.0 g, 20%. Melting Point: 188-190° C.

¹H NMR (DMSO-d₆, 400 MHz): δ 7.50-7.25 (m, 10H), 7.18 (d, J=8.8 Hz, 1H), 6.73 (d, J=2.4 Hz, 1H), 6.44 (dd, J=2.4 & 6.4 Hz, 1H), 5.10 (s, 2H), 4.18 (q, J=6.8 Hz, 1H), 3.25 (bs, 2H), 1.85-1.75 (m, 2H), 1.37 (d, J=6.8 Hz, 1H), 1.32 (s, 3H), 0.84 (t, J=7.6 Hz, 3H). Mass (CI): m/z 335 (M⁺-PEA) IR (cm⁻¹) (KBr): 3440, 2934, 1572, 1505, 1465.

### PREPARATION 63

### R-(+)-2-(3-Benzyloxy-4-chlorophenoxy)-2-methyl butyric acid methyl ester

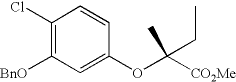

R-(+)-phenylethyl amine salt of (RS)-2-(3-benzyloxy-4-chlorophenoxy)-2-methyl butyric acid (2.0 grams, 4.30 mmol), obtained in preparation 62, was dissolved in methanol (15 mL) and concentrated sulfuric acid (0.5 mL) was drop-wise added under stirring at 20 to 40° C. The reaction mixture was refluxed for 2-3 hours, then cooled to 20 to 40° C., poured over ice-water and extracted with ethylacetate (3×15 mL). The combined organic layer was washed with water, dried over anhydrous sodium sulphate and evaporated to afford a light colored gummy mass of the title compound.

Yield: 1.45 g, 94%.

¹H NMR (200 MHz, CDCl₃) δ 7.46-7.30 (m, 5H), 7.20 (d, J=8.6 Hz, 1H), 6.53 (d, J=2.4 Hz, 1H), 6.33 (dd, J=2.8 & 6.0 Hz, 1H), 5.10 (s, 2H), 3.71 (s, 3H), 2.00-1.90 (m, 2H), 1.43 (s, 3H), 0.94 (t, J=7.6 Hz, 3H); MS (CI Method) 349 (M)⁺, 317, 289, 235, 205, 156, 114; IR (Neat) 2926, 1737, 1585, 1488 cm⁻¹.

### PREPARATION 64

### R-(+)-2-(3-Hydroxyphenoxy)-2-methyl butyric acid methyl ester

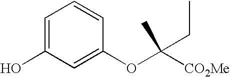

R-(+)-2-(3-benzyloxy-4-chlorophenoxy)-2-methyl butyric acid methyl ester (1.45 grams, 4.15 mmol), obtained in preparation 63, was dissolved in ethanol (10 mL) and moist palladium carbon (0.75 g, 50°/O w/w) was added. The reaction mass was hydrogenated at 40 psi pressure of hydrogen at 60-70° C. for 12 hours. The reaction mixture was cooled to 20 to 40° C. and filtered through 60-120 mesh silica gel column. After washing the column with methanol (3×20 mL), the combined reaction mixture was completely evaporated to get the above title compound. Yield: 0.85 g, 91%.

¹H NMR (CDCl₃, 200 MHz): δ 7.11 (t, J=8.0 Hz, 1H), 6.48-6.40 (m, 2H), 6.36-6.34 (m, 1H), 4.86 (bs, 1H, D₂O exchangeable), 3.77 (s, 3H), 2.25-1.85 (m, 2H), 1.51 (s, 3H), 0.96 (t, J=7.6 Hz, 3H). Mass (CI): m/z 224 (M⁺+1). IR (cm⁻¹) (KBr): 3416, 2950, 1732, 1594, 1485.

### PREPARATION 65

### Preparation of R-2-(4-chloro-3-hydroxy-phenoxy)-2-methyl butyric acid methyl ester

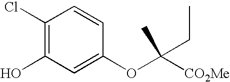

2-(3-Benzyloxy-4-chloro-phenoxy)-2-methyl butyric acid methyl ester (2.00 grams, 5.74 mmol) was dissolved in ethanol (15 mL) and moist palladium carbon (0.50 grams, 25% w/w) was added. The reaction mass was hydrogenated at 40 psi pressure of hydrogen at 20 to 40° C. for 3 hours. The reaction mixture was filtered through 60-120 mesh silica gel column. After washing the column with methanol (3×20 mL), the combined reaction mixture was completely evaporated to get the gummy mass of the title compound. Yield: 1.25 g, 85%.

¹H NMR (CDCl₃, 200 MHz): δ 7.14 (d, J=9.0 Hz, 1H), 6.53 (d, J=3.0 Hz, 1H), 6.39 (dd, J=6.0 & 2.6 Hz, 1H), 5.47 (bs, 1H, D₂O exchangeable), 3.77 (s, 3H), 2.04-1.90 (m, 2H), 1.50 (s, 3H), 0.97 (t, J=7.6 Hz, 3H). Mass (CI): m/z 259 (M⁺+1).

IR (cm⁻¹) (Neat): 3423, 2940, 1734, 1590, 1484.

### PREPARATION 66

### S-(−)-phenylethyl amine salt of (RS)-2-(3-benzyloxy-phenoxy)-2-methyl butyric acid

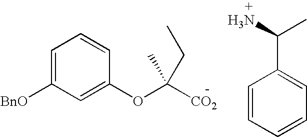

2-(3-Benzyloxy-phenoxy)-2-methyl butyric acid (7.5 grams, 25.00 mmol), obtained in preparation 56, dissolved in 20% ethylacetate-petroleum ether (75 mL), was added with S-(−)-phenylethyl amine (3.02 grams, 25.00 mmol) at 20 to 40° C., and the reaction mixture was refluxed for 1 hour. The reaction mixture was brought to 20 to 40° C. and agitated there for 1 hour. The solid separated was collected by filtration and washed with minimum volume of 20% ethylacetate-petroleum ether to get the mixture of diastereomeric salts. This solid was subjected to twenty six consecutive fractional crystallizations with a mixture of ethylacetate and petroleum ether (1:1, 3.0 vol. each time). No clear solution was observed during heating. The slurry was refluxed for 30 minutes during each crystallization and was always filtered after stirring at 20 to 40° C. for 30 minutes to finally afford the title compound. Yield: 0.5 grams. Melting Point: 186-188° C.

¹H NMR (CDCl₃, 200 MHz): δ 7.54-7.20 (m, 10H), 7.16 (t, J=8.6 Hz, 1H), 6.75 (dd, J=1.6 & 6.0 Hz, 1H), 6.62-6.55 (m, 2H), 5.05 (s, 2H), 4.15 (q, J=6.8 Hz, 1H), 3.20 (bs, 2H), 1.84-1.74 (m, 2H), 1.38 (d, J=6.8 Hz, 3H), 1.31 (s, 3H), 0.84 (t, J=7.6 Hz, 3H).

Mass (CI): m/z 301 (M⁺-PEA).

IR (cm⁻¹) (KBr): 3511, 2956, 1571, 1477.

### PREPARATION 67

### S-(−)-2-(3-Benzyloxy-phenoxy)-2-methyl butyric acid methyl ester

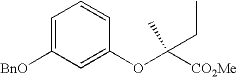

The title compound was prepared by following the same procedure as described in preparation 62 by treating S-(−)-phenylethyl amine salt of (RS)-2-(3-benzyloxy-phenoxy)-2-methyl butyric acid (0.5 grams, 1.18 mmol), obtained in preparation 66, with concentrated sulphuric acid (0.2 mL) in methanol (10 mL) at reflux temperature for 2-3 hours. Yield: 0.35 g, 94%).

¹H NMR (CDCl₃, 200 MHz): δ 7.44-7.34 (m, 5H), 7.21 (t, J=8.8 Hz, 1H), 6.77 (dd, J=1.6 & 6.0 Hz, 1H), 6.60-6.50 (m, 2H), 5.02 (s, 2H), 3.74 (s, 3H), 2.00-1.85 (m, 2H), 1.44 (s, 3H), 0.98 (t, J=7.0 Hz, 3H). Mass (CI): m/z 315 (M⁺+1).

IR (cm⁻¹) (KBr): 2944, 1739, 1582, 1490.

### PREPARATION 68

### S-(−)-2-(3-Hydroxyphenoxy)-2-methyl butyric acid methyl ester

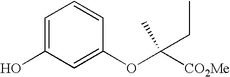

The title compound was prepared by following the same procedure as described in preparation 64 by treating S-(−)-2-(3-benzyloxy-phenoxy)-2-methyl butyric acid methyl ester (0.30 grams, 0.95 mmol), obtained in preparation 67, with moist palladium carbon (0.10 g, 33% w/w) and ethanol (10 mL). The reaction mass was hydrogenated at 40 psi pressure of hydrogen at 20 to 40° C. for 3 hours. Yield: 200 mg, 93%.

¹H NMR (CDCl₃, 200 MHz): δ 7.12 (t, J=8.0 Hz, 1H), 6.47-6.40 (m, 2H), 6.36-6.33 (m, 1H), 4.86 (bs, 1H, D₂O exchangeable), 3.76 (s, 3H), 2.25-1.85 (m, 2H), 1.51 (s, 3H), 0.96 (t, J=7.6 Hz, 3H). Mass (CI): m/z 224 (M⁺+1).

IR (cm⁻¹) (Neat): 3417, 2950, 1731, 1594, 1487.

### PREPARATION 69

### S-(−)-phenylethyl amine salt of (RS)-2-(3-benzyloxy-4-chlorophenoxy)-2-methyl butyric acid,

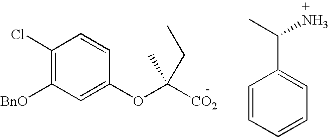

The title compound was prepared by following the same procedure as described in example 56 by treating (RS)-2-(3-benzyloxy-4-chlorophenoxy)-2-methyl butyric acid (17.0 grams, 51.08 mol) with S-(−)-phenylethyl amine (6.18 g, 51.08 mol) in 20% ethylacetate-petroleum ether (170 mL), at 20 to 40° C. for 1 hour. Yield: 5.0 grams. Melting Point: 189-190° C.

¹H NMR (DMSO-d₆, 400 MHz): δ 7.51-7.25 (m, 10H), 7.19 (d, J=8.8 Hz, 1H), 6.74 (d, J=2.4 Hz, 1H), 6.44 (dd, J=2.4 & 6.4 Hz, 1H), 5.10 (s, 2H), 4.18 (q, J=6.8 Hz, 1H), 3.25 (bs, 2H), 1.85-1.75 (m, 2H), 1.37 (d, J=6.8 Hz, 1H), 1.32 (s, 3H), 0.84 (t, J=7.6 Hz, 3H). Mass (CI): m/z 335 (M⁺-PEA).

IR (cm⁻¹) (KBr): 3442, 2935, 1572, 1505, 1468.

### PREPARATION 70

### S-(−)-2-(3-Benzyloxy-4-chlorophenoxy)-2-methyl butyric acid methyl ester

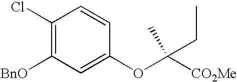

The title compound was prepared by following the same procedure as described in preparation 62 S-(−)-phenylethyl amine salt of (RS)-2-(3-benzyloxy-4-chlorophenoxy)-2-methyl butyric acid (5.0 g, 11.00 mmol), obtained in preparation 69, with concentrated sulphuric acid (1.0 mL) in methanol (25 mL) at reflux temperature for 2-3 hours. Yield: 3.40 g, 89%.

¹H NMR (CDCl₃, 200 MHz): δ 7.52-7.35 (m, 5H), 7.21 (d, J=8.6 Hz, 1H), 6.53 (d, J=2.4 Hz, 1H), 6.35 (dd, J=2.8 & 6.0 Hz, 1H), 5.10 (s, 2H), 3.71 (s, 3H), 1.99-1.86 (m, 2H), 1.43 (s, 3H), 0.94 (t, J=6.8 Hz, 3H). Mass (CI): m/z 348 (M⁺+1).

IR (cm⁻¹) (KBr): 2945, 1734, 1584, 1488.

### PREPARATION 71

### S-(−)-2-(3-Hydroxyphenoxy)-2-methyl butyric acid methyl ester

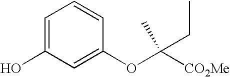

The title compound was prepared by following the same procedure as described in preparation 64 by treating S-(−)-2-(3-benzyloxy-4-chlorophenoxy)-2-methyl butyric acid methyl ester (3.35 g, 9.65 mmol), obtained in preparation 70, with moist palladium carbon (0.67 g, 20% w/w) and ethanol (15 mL). The reaction mass was hydrogenated at 40 psi pressure of hydrogen at 60 to 70° C. for 12 hours. Yield: 2.0 grams, 93%.

¹H NMR (CDCl₃, 200 MHz): δ 7.12 (t, J=8.0 Hz, 1H), 6.47-6.40 (m, 2H), 6.36-6.33 (m, 1H), 4.86 (bs, 1H, D₂O exchangeable), 3.76 (s, 3H), 2.25-1.85 (m, 2H), 1.51 (s, 3H), 0.96 (t, J=7.6 Hz, 3H). Mass (CI): m/z 224 (M⁺+1).

IR (cm⁻¹) (Neat): 3417, 2950, 1731, 1594, 1487.

### PREPARATION 72

### R-1-Naphthalen-2-yl-ethylamine salt of RS-2-(3-Hydroxy-phenoxy)-2-methyl butyric acid

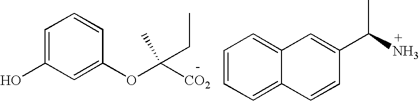

RS-2-(3-Hydroxy-phenoxy)-2-methyl butyric acid (2.50 grams, 11.90 mmol), dissolved in 20% ethylacetate-petroleum ether (15 mL), was added with R-1-naphthalen-2-yl-ethylamine (2.03 grams, 11.90 mmol) at 20 to 40° C., and the reaction mixture was refluxed for 1 hour. The reaction mixture was brought to 20 to 40° C. and agitated for 1 hour. The solid separated was collected by filtration and washed with minimum volume of 20% ethylacetate-petroleum ether to get the mixture of diastereomeric salts. This solid was subjected to three consecutive fractional crystallizations with a mixture of ethylacetate and petroleum ether (1:1, 2.0 vol. each time). Clear solution was observed during heating and solid reappeared on cooling. The mixture was refluxed for 30 minutes during each crystallization and was always filtered after stirring at 20 to 40° C. for 30 minutes to finally afford the title compound Yield: 1.0 gram Melting Point: 176-177° C.

¹H NMR (DMSO-d₆, 400 MHz): δ 7.41-7.38 (m, 2H), 7.36-7.28 (m, 5H), 6.98 (t, J=8.4 Hz, 1H), 6.50 (t, J=2.0 Hz, 1H), 6.37 (dd, J=1.0 & 4.8 Hz, 2H), 5.87 (bs, 3H), 4.18 (m, 1H), 1.87-1.74 (m, 2H), 1.38 (d, J=6.8 Hz, 3H), 1.30 (s, 3H), 0.90 (t, J=7.4 Hz, 3H).

Mass (CI): m/z 211 (M⁺-NEA). IR (cm⁻¹) (KBr): 3502, 3451, 2976, 1596, 1462.

### PREPARATION 73

### S-(−)-2-(3-Hydroxyphenoxy)-2-methyl butyric acid methyl ester

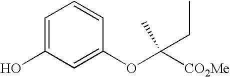

R-1-Naphthalen-2-yl-ethylamine salt of RS-2-(3-Hydroxy-phenoxy)-2-methyl butyric acid (1.00 grams, 2.62 mmol) was dissolved in methanol (25 mL) and concentration sulfuric acid (0.5 mL) was drop-wise added under stirring at 20 to 40° C. Refluxed the reaction mixture for 2-3 hours then cooled the reaction mixture to 20 to 40° C., poured over ice-water and extracted with ethylacetate (3×20 mL). The combined organic layer was washed with water, dried over anhydrous sodiumsulphate and evaporated to afford a light colored gummy mass of the title compound.

Yield: 0.49 grams, 83%.

¹H NMR (CDCl₃, 200 MHz): δ 7.12 (t, J=8.0 Hz, 1H), 6.47-6.40 (m, 2H), 6.36-6.33 (m, 1H), 4.86 (bs, 1H, D₂O exchangeable), 3.76 (s, 3H), 2.25-1.85 (m, 2H), 1.51 (s, 3H), 0.96 (t, J=7.6 Hz, 3H). Mass (CI): m/z 224 (M⁺+1). IR (cm⁻¹) (Neat): 3417, 2950, 1731, 1594, 1487.

### PREPARATION 74

### S-(−)-2-(4-chloro-3-hydroxyphenoxy)-2-methyl butyric acid methyl ester

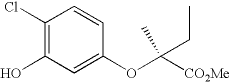

S-2-(3-Benzyloxy-4-chloro-phenoxy)-2-methyl butyric acid methyl ester (1.8 grams, 5.15 mmol) was dissolved in ethanol (20 mL) and moist palladium carbon (0.45 grams, 25% w/w) was added. The reaction mass was hydrogenated at 40 psi pressure of hydrogen at 20 to 40° C. for 3 hours. The reaction mixture was filtered through 60-120 mesh silica gel column. After washing the column with methanol (3×20 mL), the combined reaction mixture was completely evaporated to get the gummy mass of the title product. Yield: 1.20 grams, 90%.

¹H NMR (CDCl₃, 200 MHz): δ 7.15 (d, J=9.0 Hz, 1H), 6.54 (d, J=3.0 Hz, 1H), 6.40 (dd, J=6.0 & 2.6 Hz, 1H), 5.50 (bs, 1H, D₂O exchangeable), 3.78 (s, 3H), 2.00-1.90 (m, 2H), 1.50 (s, 3H), 0.98 (t, J=7.6 Hz, 3H). Mass (CI): m/z 259 (M⁺).

IR (cm⁻¹) (Neat): 3424, 2940, 1734, 1591, 1484

### ALTERNATE PROCESS FOR PREPARATION 63

### R-(+)-2-(3-Benzyloxy-4-chlorophenoxy)-2-methyl butyric acid methyl ester

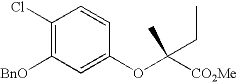

### Step (i):

### R-1-Naphthalen-1-yl-ethylamine salt of RS-2-hydroxy-2-methyl butyric acid 29 and its resolution to pure 30

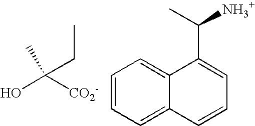

RS-2-hydroxy-2-methyl butyric acid (6.00 grams, 50.80 mmol), dissolved in acetone (24 mL), was added with R-1-naphthalen-1-yl-ethylamine (8.20 mL, 50.80 mmol) at 25-35° C., and the reaction mixture was refluxed for 2 hours. The reaction mixture was brought to 25-35° C. and kept in fridge for 9-12 hours. The solid separated was collected by filtration and washed with minimum volume of acetone to get the mixture of diastereomeric salts. This solid was subjected to two more consecutive fractional crystallizations as above with minimum volume of acetone (preferably two vol. each time) to finally afford the desired single diastereomeric salt (4.80 grams, 33%). HPLC (chiral) 99.08% [column: chiralpak AD; mobile phase: Hexane:IPA:TFA (96:04:0.1); UV: 215 nm].

### Step (ii)

### S-2-Hydroxy-2-methyl butyric acid methyl ester

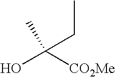

The diastereomeric salt (2.00 grams, 6.90 mmol), obtained in above step (i), was dissolved in methanol (30 mL) and conc. H₂SO₄ (0.5 mL) was drop-wise added under stirring at 25-35° C. Refluxed the reaction mixture for 2-3 hours and evaporated the solvent. After bringing to 25-35° C., the reaction mixture was added with ethylacetate (20 mL) and extracted with minimum volume of water and aqueous sodium bicarbonate. The organic layer was finally washed with minimum volume of water, dried (anhydydrous sodium sulphate) and evaporated to afford a light brown gummy mass of the title compound (0.85 grams, 92%).

¹H NMR (200 MHz, CDCl₃) δ 3.78 (s, 3H), 3.19 (bs, 1H), 1.84-1.61 (m, 2H), 1.40 (s, 3H), 0.97 (t, J=7.6 Hz, 3H). MS (CI Method) 132 (M⁺), 114;

IR (Neat) 3442, 2924, 1731, 1462 cm⁻¹.

### Step (iii)

### S-2-Methanesulfonyloxy-2-methyl butyric acid methyl ester

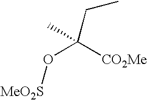

S-2-Hydroxy-2-methyl butyric acid methyl ester (0.92 grams, 6.90 mmol), obtained in the above step (ii), was dissolved in dichloromethane (5 mL) and triethylamine (2.4 mL, 17.4 mmol) was drop-wise added under stirring at 25-35° C. After cooling the reaction mixture to 0-5° C., methane sulfonyl chloride (0.80 mL, 10.40 mmol) was drop-wise added and allowed the reaction to run at room temperature for 8 hours. Dichloromethane (10 mL) was added to the reaction mixture and the content was extracted with minimum volume of water and brine solution. The combined organic layer was finally washed with minimum volume of water, dried (anhydrous sodium sulphate) and evaporated to afford a light brown gummy mass. This mass was purified by column chromatography using 230-400 mesh silica-gel and mixture of ethyl acetate and petroleum ether (90:10) to afford a white gummy mass of the title compound (400 mg, 30%).

¹H NMR (200 MHz, CDCl₃) δ 3.81 (s, 3H), 3.14 (s, 3H), 2.01-1.89 (m, 2H), 1.78 (s, 3H), 0.97 (t, J=7.4 Hz, 3H). MS (CI Method) 211 (M⁺1)⁺, 114;

IR (Neat) 2950, 1747, 1463, 1348 cm⁻¹.

### Step (iv)

### R-2-(3-Benzyloxy-4-chlorophenoxy)-2-methyl butyric acid methyl ester

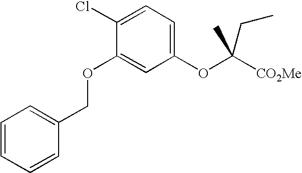

3-Benzyloxy-4-chlorophenol (200 mg, 0.85 mmol), dissolved in toluene (15 mL), was added with anhyd. Potassium carbonate (353 mg, 2.50 mmol) at room temperature under stirring. S-2-Methanesulfonyloxy-2-methyl butyric acid methyl ester (233 mg, 1.10 mmol), obtained in the above step (iii), and benzyltriethylammonium bromide (BTEAB, 46 mg, 0.17 mmol) were subsequently added to the reaction mixture. The reaction mixture was refluxed for 9-12 hours. After cooling to 25-35° C., the reaction mixture was poured over ice-water, acidified with 6 N HCl and extracted with ethyl acetate (3×15 mL). The combined organic layer was washed with water, dried (anhydrous Na₂SO₄) and evaporated to obtain a gummy mass which was purified by column chromatography using 230-400 mesh silica-gel and mixture of ethyl acetate and petroleum ether (80:20) to afford light brown colored title compound (145 mg, 43%).

¹H NMR (200 MHz, CDCl₃) δ 7.46-7.30 (m, 5H), 7.20 (d, J=8.6 Hz, 1H), 6.53 (d, J=2.4 Hz, 1H), 6.33 (dd, J=2.8 & 6.0 Hz, 1H), 5.10 (s, 2H), 3.71 (s, 3H), 2.00-1.90 (m, 2H), 1.43 (s, 3H), 0.94 (t, J=7.6 Hz, 3H);

MS (CI Method) 349 (M)⁺, 317, 289, 235, 205, 156, 114;

IR (Neat) 2926, 1737, 1585, 1488 cm⁻¹.

### EXAMPLE 1

### 2-Methyl-2-{4-[2-(2-methyl-4-oxo-4H-quinazolin-3-yl)-ethylamino]-phenylsulfanyl}-propionic acid ethyl ester

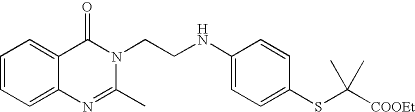

To a solution of 3-(2-chloroethyl)-2-methyl-3,4-dihydro-4-quinazolinone (0.8 grams, 3.5 mmol), obtained in preparation 4, in toluene (100 mL) ethyl 2-(4-aminophenylsulfanyl)-2-methylpropanoate (0.771 grams, 3.1 mmol) was added followed by the addition of potassium carbonate (483 mg, 3.42 mmol) and potassium iodide (1.0 gram). The reaction mixture was heated to 130-140° C. for 8 hours. Mixture was then concentrated and after workup using ethyl acetate gave crude mass, which was then purified by column chromatography on basic alumina using 95% ethyl acetate in hexane to yield pure title compound. Yield: 0.5 grams, 26%.

¹H NMR (CDCl₃, 300 MHz): δ 1.23 (t, J=7.1 Hz, 3H), 1.43 (s, 6H), 2.61 (s, 3H), 3.58 (q, J=6 Hz, 2H), 4.11 (q, J=7.1 Hz, 3H), 4.36 (t, J=6.1 Hz, 2H), 6.58 (d, J=8.7 Hz, 2H), 7.27 (d, J=8.6 Hz, 2H), 7.46 (t, J=8.1 Hz, 1H), 7.61 (d, J=7.7 Hz, 1H), 7.70-7.75 (m, 1H), 8.26 (dd, J=1.1 & 8.0 Hz, 1H).

### EXAMPLE 2

### 2-Methyl-2-{4-[2-(2-methyl-4-oxo-4H-quinazolin-3-yl)-ethylamino]-phenylsulfanyl}-propionic acid

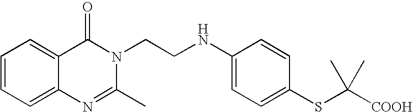

To a solution of 2-methyl-2-{4-[2-(2-methyl-4-oxo-4H-quinazolin-3-yl)-ethylamino]-phenylsulfanyl}-propionic acid ethyl ester (475 mg, 1.09 mmol), obtained in example 1, in methanol (20 mL) lithium hydroxide (266 mg, 10.92 mmol) in water (5 mL) was added. The reaction mixture was stirred at 20 to 40° C. for 12 to 17 hours. Reaction mixture was then concentrated, ethyl acetate (50 mL) and water (50 mL) were added and stirred for 5 minutes. Separated organic layer was removed and the aqueous layer was acidified (pH ˜5) by using acetic acid at 0-5° C. The product was extracted with ethyl acetate (100 mL). Ethyl acetate layer was dried over sodium sulfate and concentrated. The crude mass was purified by chromatography on a 60-120 mesh silica gel column using 1.5% methanol in chloroform to afford the title compound

Yield: 330 mg, 74.6%.

Melting Point: 226-228° C.

¹H NMR (CDCl₃, 300 MHz): δ 1.47 (s, 6H), 2.61 (s, 3H), 3.62 (t, J=6.0 Hz, 2H), 4.37 (t, J=6.0 Hz, 2H), 6.58 (d, J=8.7 Hz, 2H), 7.32 (d, J=8.7 Hz, 2H), 7.49 (t, J=8.1 Hz, 1H), 7.66 (d, J=7.5 Hz, 1H), 7.75-7.80 (m, 1H), 8.23 (dd, J=1.2 & 8.1 Hz, 1H).

Mass (ES): m/z 398 (M⁺+1).

### EXAMPLE 3

### 2-{4-[2-(2-Ethyl-4-oxo-4H-quinazolin-3-yl)-ethylamino]-phenylsulfanyl}-2-methyl-propionic acid ethyl ester

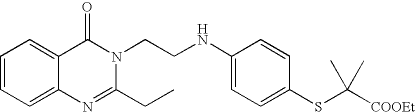

The title compound was prepared by following the same procedure as described in the example 1 by heating ethyl 2-(4-aminophenylsulfanyl)-2-methylpropanoate (2.01 grams, 8.2 mmol) and 3-(2-chloroethyl)-2-ethyl-3,4-dihydro-4-quinazolinone (2 grams, 8.2 mmol), obtained in preparation 7, in the presence of potassium carbonate (3.51 grams, 29 mmol) and potassium iodide (1.0 grain) in toluene (100 mL.) at 135° C. for 48 hours.

Yield: 8.3 grams, 99%.

¹H NMR (CDCl₃, 300 MHz): δ 1.16 (t, J=7.1 Hz, 3H), 1.31 (t, J=7.3 Hz, 3H), 1.35 (s, 6H), 2.76 (q, J=7.3 Hz, 2H), 3.46-3.52 (m, 2H), 4.04 (q, J=7.1 Hz, 2H), 4.30 (t, J=6.3 Hz, 2H), 6.51 (d, J=8.6 Hz, 2H), 7.19 (d, J=8.6 Hz, 2H), 7.39 (t, J=8.0 Hz, 1H), 7.58 (d, J=8.0 Hz, 2H), 8.2 (d, J=8.0 Hz, 1H).

### EXAMPLE 4

### 2-{4-[2-(2-Ethyl-4-oxo-4H-quinazolin-3-yl)-ethylamino]-phenylsulfanyl}-2-methyl-propionic acid

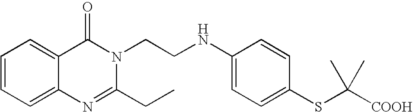

The title compound was prepared by following the same procedure as described in example 2 by hydrolyzing 2-{4-[2-(2-ethyl-4-oxo-4H-quinazolin-3-yl)-ethylamino]-phenylsulfanyl}-2-methyl-propionic acid ethyl ester (0.525 grams, 1.14 mmol), obtained in example 3, in methanol-water (10 mL, 1:1) using lithium hydroxide (1.4 grams, 57.16 mmol) at 20 to 40° C. for 12 to 17 hours.

Yield: 166 mg, 32.6%.

Melting Point: 124-126° C.

¹H NMR (DMSO-d₆, 300 MHz): δ 1.22 (t, J=7.2 Hz, 3H), 1.3 (s, 6H), 2.82 (q, J=7.2 Hz, 2H), 3.37-3.45 (m, 2H), 4.17 (t, J=6.7 Hz, 2H), 6.3 (t, J=6.2 Hz, 1H, D₂O exchangeable), 6.63 (d, J=8.5 Hz, 2H), 7.16 (d, J=8.5 Hz, 2H), 7.49 (t, J=7.5 Hz, 1H), 7.61 (d, J=8.0 Hz, 2H), 7.79 (t, J=8.3 Hz, 1H), 8.15 (d, J=8.0 Hz, 2H).

Mass (ES): m/z 412 (M⁺+1).

### EXAMPLE 5

### 2-Methyl-2-{4-[2-(4-oxo-2-propyl-4H-quinazolin-3-yl)-ethylamino]-phenylsulfanyl}-propionic acid ethyl ester

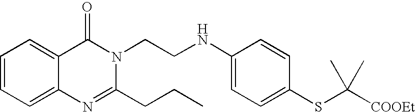

The title compound was prepared by following the same procedure as described in example 1 by refluxing ethyl 2-(4-aminophenylsulfanyl)-2-methylpropanoate (787 mg, 3.22 mmol) and 3-(2-chloroethyl)-2-propyl-3,4-dihydro-4-quinazolinone (1.156 grams 4.53 mmol), obtained in preparation 10, in the presence of Potassium carbonate (1.92 gm, 13.5 mmol) and potassium iodide (100 mg) in toluene (100 mL) for 48 hours.

Yield: 0.5 grams, 24%.

¹H NMR (CDCl₃, 300 MHz): δ 0.94 (t, J=7.4 Hz, 3H), 1.15 (t, J=7.2 Hz, 2H), 1.35 (s, 6H), 1.7-1.82 (m, 2H), 2.67 (t, J=7.8 Hz, 2H), 3.48 (t, J=6.0 Hz, 2H), 4.03 (q, J=7.1 Hz, 2H), 4.28 (t, J=6.2 Hz, 2H), 6.51 (d, J=8.5 Hz, 2H), 7.19 (d, J=8.5 Hz, 2H), 7.23 (t, J=7.9 Hz, 1H), 7.55 (d, J=7.9 Hz, 1H), 7.65 (t, J=8.3 Hz, 1H), 8.18 (d, J=7.9 Hz, 1H).

### EXAMPLE 6

### 2-Methyl-2-{4-[2-(4-oxo-2-propyl-4H-quinazolin-3-yl)-ethylamino]-phenylsulfanyl}-propionic acid

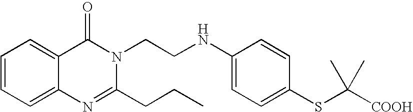

The title compound was prepared by following the same procedure as described in example 2, by hydrolyzing the compound obtained from 2-methyl-2-{4-[2-(4-oxo-2-propyl-4H-quinazolin-3-yl)-ethylamino]-phenylsulfanyl}-propionic acid ethyl ester (1.1 grams, 2.3 mmol), obtained in example 5, in ethanol-water (16 mL, 11:5) using lithium hydroxide (2.32 grams, 94.7 mmol) at 20 to 40° C. for 12 to 17 hours. Yield: 200 mg, 20%. Melting Point: 162-164° C.

¹H NMR (DMSO-d₆, 300 MHz): δ 0.87 (t, J=7.4 Hz, 3H), 1.28 (s, 6H), 1.71 (q, J=7.4 Hz, 2H), 2.70 (t, J=7.5 Hz, 2H), 3.4 (t, J=6.2 Hz, 2H), 4.14 (t, J=6.3 Hz, 2H), 6.3 (t, J=6.2 Hz, 1H, D₂O exchangeable), 6.61 (d, J=8.5 Hz, 2H), 7.15 (d, J=8.4 Hz, 2H), 7.47 (t, J=7.2 Hz, 2H), 7.57 (d, J=8.0 Hz, 2H), 7.76 (t, J=7 Hz, 2H), 8.12 (d, J=7.9 Hz, 2H). Mass (ES): m/z 426 (M⁺+1).

### EXAMPLE 7

### 2-Methyl-2-{4-[3-(2-methyl-4-oxo-4H-quinazolin-3-yl)-propylamino]-phenyl sulfanyl}-propionic acid ethyl ester

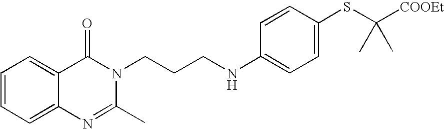

The title compound was prepared by following the same procedure as described in example 1 by refluxing ethyl-2-(4-aminophenylsulfanyl)-2-methylpropanoate (0.401 grams, 1.64 mmol), obtained in preparation 14, and 3-(3-chloropropyl)-2-methyl-3,4-dihydro-4-quinazolinone (0.5 grams, 1.52 mmol) in the presence of potassium carbonate (1.27 grams, 8.92 mmol) and potassium iodide (500 mg.) in toluene (100 mL) for 48 hours. Yield: 0.4 grams, 43%.

¹H NMR (CDCl₃, 300 MHz): δ 1.16 (t, J=7.1 Hz, 3H), 1.37 (s, 6H), 1.93-2.02 (m, 2H), 2.56 (s, 3H), 3.17 (t, J=6.2 Hz, 2H), 4.04 (q, J=7.1 Hz, 2H), 4.15 (t, J=7.1 Hz, 1H), 6.49 (d, J=8.6 Hz, 2H), 7.19 (d, J=8.6 Hz, 2H), 7.38 (t, J=8.1 Hz, 1H), 7.54 (d, J=7.7 Hz, 1H), 7.63-7.70 (m, 1H), 8.18 (dd, J=1.10 & 8.0 Hz, 1H).

### EXAMPLE 8

### 2-Methyl-2-{4-[3-(2-methyl-4-oxo-4H-quinazolin-3-yl)-propylamino]-phenyl sulfanyl}-propionic acid

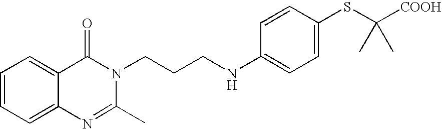

The title compound was prepared by following the same procedure as described in example 2 by treating 2-methyl-2-{4-[3-(2-methyl-4-oxo-4H-quinazolin-3-yl)-propylamino]-phenylsulfanyl}-propionic acid ethyl ester (0.7 grams, 1.56 mmol), obtained in example 7, with lithium hydroxide (0.991 grams, 40.4 mmol) in ethanol-water (12 mL, 5:1) at 20 to 40° C. for 12 to 17 hours. Yield: 400 mg, 60%. Melting Point: 162-164° C.

¹H NMR (DMSO-d₆, 300 MHz): δ 1.3 (s, 6H), 1.89-1.99 (m, 2H), 2.58 (s, 3H), 3.13 (m, 2H), 4.13 (t, J=7.6 Hz, 2H), 6.09 (t, J=6.2 Hz, 1H, D₂O exchangeable), 6.55 (d, J=8.5 Hz, 2H), 7.15 (d, J=8.5 Hz, 2H), 7.47 (t, J=7.5 Hz, 1H), 7.57 (d, J=8.1 Hz, 1H), 7.78 (t, J=7.5 Hz, 1H), 8.10 (d, J=8.0 Hz, 2H). Mass (ES): m/z 412 (M⁺+1).

### EXAMPLE 9

### 2-{4-[3-(2-Ethyl-4-oxo-4H-quinazolin-3-yl)-propylamino]-phenylsulfanyl}-2-methyl-propionic acid ethyl ester

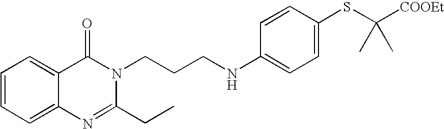

The title compound was prepared by following the same procedure as described in example 1 by refluxing ethyl 2-(4-aminophenylsulfanyl)-2-methylpropanoate (1.92 grams, 7.87 mmol) and the 3-(3-chloropropyl)-2-ethyl-3,4-dihydro-4-quinazolinone (2.0 grams, 7.82 mmol), obtained in preparation 17, in the presence of potassium carbonate (3.32 grams 23.4 mmol) and potassium iodide (800 mg) in toluene (100 mL) for 72 hours. Yield: 0.2 grams, 5.5%

¹H NMR (CDCl₃, 300 MHz): δ 1.2 (t, 3H), 1.15 (t, 3H), 1.35 (s, 6H), 2.0 (quin, 2H), 2.8 (q, 2H), 3.2 (t, 2H), 4.01 (m, 4H), 4.4 (brs, 1H, D₂O exchangeable), 6.5 (dd, J=1.9 & 6.7 Hz, 2H), 7.2 (m, 2H), 7.4 (m, 1H), 7.5 (m, 1H), 7.6 (m, 1H), 8.2 (dd, J=1.1 & 8.1 Hz, 1H).

### EXAMPLE 10

### 2-{4-[3-(2-Ethyl-4-oxo-4H-quinazolin-3-yl)-propylamino]-phenylsulfanyl}-2-methyl-propionic acid

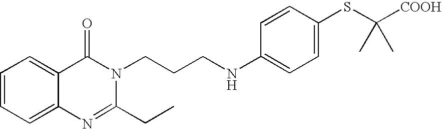

The title compound was prepared by following the same procedure as described in example 2 by treating 2-{4-[3-(2-ethyl-4-oxo-4H-quinazolin-3-yl)-propylamino]-phenylsulfanyl}-2-methyl-propionic acid ethyl ester (0.55 grams, 1.09 mmol), obtained in example 9, with lithium hydroxide (1.45 grams, 59 mmol) in ethanol-water (12 mL, 5:1) at 20 to 40° C. for 12 to 17 hours. Yield: 400 mg, 60%. Melting Point: 60-62° C.

¹H NMR (CDCl₃, 300 MHz): δ 1.25 (t, J=7.1 Hz, 3H), 1.30 (s, 6H), 1.87-1.97 (m, 2H), 2.86 (q, J=7.3 Hz, 2H), 3.11-3.17 (m, 2H), 4.14 (t, J=7.6 Hz, 2H), 6.09 (t, J=5.2 Hz, 1H, D₂O exchangeable), 6.55 (d, J=8.5 Hz, 2H), 7.15 (d, J=8.5 Hz, 2H), 7.47 (t, J=7.5 Hz, 1H), 7.6 (d, J=8.1 Hz, 1H), 7.78 (t, J=8.3 Hz, 1H), 8.1 (dd, J=1.0 & 8.1 Hz, 1H). Mass (ES): m/z 426 (M⁺+1).

### EXAMPLE 11

### 2-Methyl-2-{4-[3-(4-oxo-2-propyl-4H-quinazolin-3-yl)-propylamino]-phenyl sulfanyl}-propionic acid ethyl ester

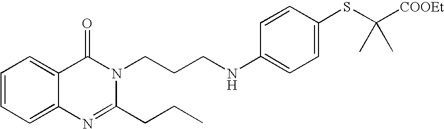

The title compound was prepared by following the same procedure as described in example 1 by refluxing ethyl 2-(4-aminophenylsulfanyl)-2-methylpropanoate (946 mg 3.87, mmol) and 3-(3-chloropropyl)-2-propyl-3,4-dihydro-4-quinazolinone (1.0 grams, 3.59 mmol), obtained in preparation 20, in the presence of potassium carbonate (1.49 grams, 10.5 mmol) and potassium iodide (400 mg.) in toluene (50 mL.) for 8 hours.

Yield: 0.6 grams, 34%.

¹H NMR (CDCl₃, 300 MHz): δ 0.98 (t, J=7.3 Hz, 3H), 1.17 (t, J=7.1 Hz, 3H), 1.37 (s, 6H), 1.73-1.86 (m, 3H), 1.93-2.04 (m, 2H), 2.71 (t, J=7.5 Hz, 2H), 3.17 (t, J=6.1 Hz, 2H), 4.05 (q, J=7.1 Hz, 2H), 4.16 (t, J=7.1 Hz, 2H), 6.5 (d, J=8.6 Hz, 2H), 7.2 (d, J=8.5 Hz, 2H), 7.38 (t, J=8.0 Hz, 1H), 7.57 (d, J=8.0 Hz, 1H), 7.63-7.69 (m, 1H), 8.2 (dd, J=0.9 & 7.9 Hz, 1H).

### EXAMPLE 12

### 2-Methyl-2-{4-[3-(4-oxo-2-propyl-4H-quinazolin-3-yl)-propylamino]-phenyl sulfanyl}-propionic acid

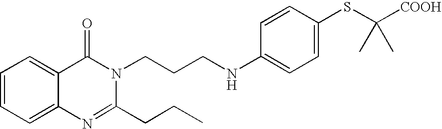

The title compound was prepared by following the same procedure as described in example 2 by using 2-methyl-2-{4-[3-(4-oxo-2-propyl-4H-quinazolin-3-yl)-propylamino]-phenylsulfanyl}-propionic acid ethyl ester (0.2 grams, 0.42 mmol), obtained in example 11, and lithium hydroxide (512 mg 20.9 mmol) in ethanol-water (30 mL, 5:1) at 20 to 40° C. for 12 to 17 hours. Yield: 400 mg, 60%

¹H NMR (DMSO-d₆, 300 MHz): δ 0.94 (t, J=7.3 Hz, 3H), 1.23 (s, 6H), 1.70-1.82 (m, 2H), 1.88-1.98 (m, 2H), 2.77 (t, J=7.5 Hz, 2H), 3.10-3.18 (m, 2H), 4.13 (t, J=7.5 Hz, 2H), 6.12 (t, J=5.2 Hz, 1H, D₂O exchangeable), 6.55 (d, J=8.6 Hz, 2H), 7.15 (d, J=8.5 Hz, 2H), 7.47 (t, J=8.0 Hz, 1H), 7.58 (d, J=7.8 Hz, 1H), 7.77 (t, J=8.4 Hz, 1H), 8.1 (dd, J=1.2 & 8.0 Hz, 1H).

Mass (ES): m/z 440 (M⁺+1).

### EXAMPLE 13

### 2-Methyl-2-{4-[4-(2-methyl-4-oxo-4H-quinazolin-3-yl)-butylamino]-phenylsulfanyl}-propionic acid ethyl ester

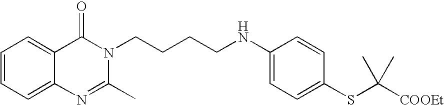

The title compound was prepared by following the same procedure as described in example 1 by heating ethyl-2-(4-aminophenylsulfanyl)-2-methylpropanoate (1.9 grams, 7.8 mmol) and 3-(4-chlorobutyl)-2-methyl-3,4-dihydro-4-quinazolinone (2.0 grams, 7.8 mmol), obtained in preparation 24, in the presence of potassium carbonate (3.31 grams, 23.4 mmol) and tetrabutylammonium bromide (513 mg, 1.56 mmol) in toluene (75 mL.) at 135-140° C. for 48 hours. Yield: 1.3 grams, 36.1%.

¹H NMR (CDCl₃, 300 MHz): δ 1.15 (t, J=7.1 Hz, 3H), 1.36 (s, 6H), 1.58-1.82 (m, 4H), 2.57 (s, 3H), 3.15 (t, J=6.6 Hz, 2H), 4.0-4.10 (m, 4H), 6.45 (d, J=8.5 Hz, 2H), 7.18 (d, J=8.9 Hz, 2H), 7.37 (t, J=7.5 Hz, 1H), 7.53 (d, J=8.0 Hz, 1H), 7.65 (t, J=8.3 Hz, 1H), 8.18 (d, J=7.9 Hz, 1H).

### EXAMPLE 14

### 2-Methyl-2-{4-[4-(2-methyl-4-oxo-4H-quinazolin-3-yl)-butylamino]-phenylsulfanyl}-propionic acid

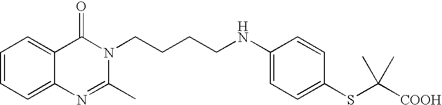

The title compound was prepared by following the same procedure as described in example 2 by using 2-methyl-2-{4-[4-(2-methyl-4-oxo-4H-quinazolin-3-yl)-butylamino]-phenylsulfanyl}-propionic acid ethyl ester (1.2 grams, 2.6 mmol), obtained in example 13, and lithium hydroxide (3.17 grams, 129.8 mmol) in ethanol-water (18 mL, 2:1) at 20 to 40° C. for 12 to 17 hours. Yield: 750 mg, 66%. Melting Point: 100-102° C.

¹H NMR (DMSO-d₆, 300 MHz): δ 1.3 (s, 6H), 1.59-1.80 (m, 4H), 2.62 (s, 3H), 3.03-3.09 (m, 2H), 4.08 (t, J=7.5 Hz, 2H), 6.02 (t, J=5.2 Hz, 1H), 6.51 (d, J=8.6 Hz, 2H), 7.13 (d, J=8.6 Hz, 2H), 7.47 (t, J=8.0 Hz, 1H), 7.58 (d, J=8.0 Hz, 1H), 7.78 (t, J=8.4 Hz, 1H), 8.1 (dd, J=1.3 & 7.9 Hz, 1H). Mass (ES): m/z 426 (M⁺+1).

### EXAMPLE 15

### 2-{4-[4-(2-Ethyl-4-oxo-4H-quinazolin-3-yl)-butylamino]-phenylsulfanyl}-2-methyl-propionic acid ethyl ester

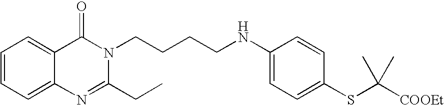

The title compound was prepared by following the same procedure as described in example 1 by heating ethyl 2-(4-aminophenylsulfanyl)-2-methylpropanoate (1.62 grams, 6.65 mmol) and 3-(4-chlorobutyl)-2-ethyl-3,4-dihydro-4-quinazolinone (1.8 grams, 6.65 mmol), obtained in preparation 27, in the presence of potassium carbonate (2.83 grams, 20 mmol) and tetrabutylammonium bromide (437 mg) in toluene (50 mL) at 135° C. for 72 hours. Yield: 487 mg 15.3%.

¹H NMR (CDCl₃, 300 MHz): δ 1.23 (t, J=7.1 Hz, 3H), 1.41 (t, J=7.4 Hz, 3H), 1.44 (s, 6H), 1.72-1.89 (m, 4H), 2.86 (q, J=7.4 Hz, 2H), 3.22 (t, J=6.6 Hz, 2H), 4.08-4.17 (m, 4H), 6.52 (d, J=7.5 Hz, 2H), 7.25 (d, J=7.6 Hz, 2H), 7.44 (t, J=8.1 Hz, 1H), 7.64 (d, J=7.5 Hz, 1H), 7.70-7.79 (m, 1H), 8.25 (dd, J=1.1 & 7.9 Hz, 1H).

### EXAMPLE 16

### 2-{4-[4-(2-Ethyl-4-oxo-4H-quinazolin-3-yl)-butylamino]-phenylsulfanyl}-2-methyl-propionic acid

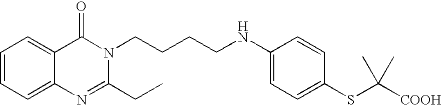

The title compound was prepared by following the same procedure as described in example 2 by using 2-{4-[4-(2-ethyl-4-oxo-4H-quinazolin-3-yl)-butylamino]-phenylsulfanyl}-2-methyl-propionic acid ethyl ester (0.487 grams, 1.04 mmol), obtained in example 15, and lithium hydroxide (1.24 grams, 51.8 mmol) in ethanol-water (12 mL, 5:1) at 20 to 40° C. for 12 to 17 hours. Yield: 750 mg, 66%. Melting Point: 58-62° C.

¹H NMR (DMSO-d₆, 300 MHz): δ 1.24-1.31 (m, 9H), 1.58-1.79 (m, 4H), 2.91 (q, J=7.2 Hz, 2H), 3.04-3.09 (m, 2H), 4.08 (t, J=7.4 Hz, 2H), 6.51 (d, J=8.5 Hz, 2H), 7.13 (d, J=8.5 Hz, 2H), 7.47 (t, J=7.3 Hz, 1H), 7.6 (d, J=8.0 Hz, 1H), 7.78 (t, J=8.3 Hz, 1H), 8.1 (d, J=7.0 Hz, 1H). Mass (ES): m/z 440 (M⁺+1).

### EXAMPLE 17

### 2-Methyl-2-{4-[4-(4-oxo-2-propyl-4H-quinazolin-3-yl)-butylamino]-phenylsulfanyl}-propionic acid ethyl ester

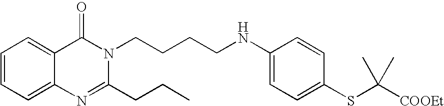

The title compound was prepared following the same procedure as described in the example 1 by heating ethyl-2-(4-aminophenylsulfanyl)-2-methylpropanoate (1.46 grams, 6.0 mmol) and 3-(4-chlorobutyl)-2-propyl-3,4-dihydro-4-quinazolinone (1.7 grams, 6 mmol), obtained in preparation 30, in the presence of potassium carbonate (2.83 grams, 20 mmol) and tetrabutylammonium bromide (394 mg) in toluene (50 mL) at 135° C. for 8 hours. Yield: 530 mg, 18%.

¹H NMR (CDCl₃, 300 MHz): δ 1.08 (t, J=7.3 Hz, 3H), 1.24 (t, J=7.1 Hz, 3H), 1.44 (s, 6H), 1.71-1.94 (m, 6H), 2.79 (t, J=7.7 Hz, 2H), 3.23 (t, J=6.6 Hz, 2H), 4.08-4.17 (m, 4H), 6.53 (d, J=8.6 Hz, 2H), 7.25 (d, J=7.6 Hz, 2H), 7.44 (t, J=7.5 Hz, 1H), 7.63 (d, J=7.8 Hz, 1H), 7.72 (t, J=8.3 Hz, 1H), 8.25 (d, J=6.8 Hz, 1H).

### EXAMPLE 18

### 2-Methyl-2-{4-[4-(4-oxo-2-propyl-4H-quinazolin-3-yl)-butylamino]-phenylsulfanyl}-propionic acid

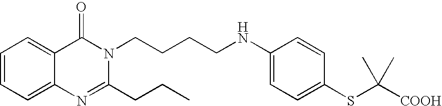

The title compound was prepared following the same procedure as described in example 2 by using 2-methyl-2-{4-[4-(4-oxo-2-propyl-4H-quinazolin-3-yl)-butylamino]-phenylsulfanyl}-propionic acid ethyl ester (0.5 grams, 1.0 mmol), obtained in example 17, and lithium hydroxide (1.31 grams, 54.7 mmol) in ethanol-water (12 mL, 5:1) at 20 to 40° C. for 12 to 17 hours. Yield: 203 mg, 42%. Melting Point 100-104° C.

¹H NMR (DMSO-d₆, 300 MHz): δ 0.99 (t, J=7.3 Hz, 3H), 1.29 (s, 6H), 1.60-1.86 (m, 6H), 2.83 (t, J=7.4 Hz, 2H), 3.06 (q, J=5.2 Hz, 2H), 4.08 (t, J=7.4 Hz, 2H), 6.51 (d, J=8.5 Hz, 2H), 7.13 (d, J=8.4 Hz, 2H), 7.47 (t, J=7.9 Hz, 1H), 7.59 (d, J=8.1 Hz, 1H), 7.78 (t, J=8.3 Hz, 1H), 8.1 (dd, J=1.3 & 7.9 Hz, 1H). Mass (ES): m/z 454 (M⁺+1).

### EXAMPLE 19

### 2-{4-[2-(5-Ethyl-1-methyl-7-oxo-3-propyl-1,7-dihydro-pyrazolo[4,3-d]pyrimidin-6-yl)-ethoxy]-phenylamino}-propionic acid ethyl ester

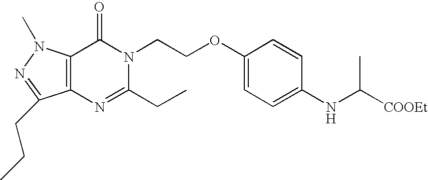

A mixture of 6-[2-(4-aminophenoxy)ethyl]-5-ethyl-1-methyl-3-propyl-6,7-dihydro-1H-pyrazolo[4,3-d]pyrimidin-7-one (0.2 grams, 0.563 mmol), obtained in preparation 49, and potassium carbonate (194 mg, 1.41 mmol) in dry dimethylformamide (4 mL) was stirred at 20 to 40° C. for 30 minutes. Ethyl-2-bromo propionate (88 μL) was added slowly drop wise and was stirred 12 to 17 hours at 20 to 40° C. Water (50 mL) was added and was extracted with ethyl acetate thrice. Combined organic extracts were washed with water, brine, dried over anhydrous sodium sulfate and evaporated under reduced pressure. Crude product was purified on a 100-200 mesh silica gel column using 15% ethyl acetate in pet ether to afford the desired compound as off white solid.

Yield: 180 mg, 70%. Melting Point: 104-108° C.

¹H NMR (CDCl₃, 200 MHz): δ 0.99 (t, J=7.3 Hz, 3H), 1.23 (t, J=7.1 Hz, 3H), 1.30-1.42 (m, 6H), 1.71-1.90 (m, 2H), 2.85 (t, J=7.6 Hz, 2H), 3.03 (q, J=7.3 Hz, 2H), 4.10-4.24 (m, 8H), 4.46 (t, J=5.1 Hz, 2H), 6.54 (d, J=8.8 Hz, 2H), 6.7 (d, J=8.8 Hz, 2H).

Mass (EI): m/z 456 (M⁺+1).

### EXAMPLE 20

### 2-{4-[2-(5-Ethyl-1-methyl-7-oxo-3-propyl-1,7-dihydro-pyrazolo[4,3-d]pyrimidin-6-yl)-ethoxy]-phenylamino}-propionic acid methyl ester (20A) and

### 2-{4-[2-(5-Ethyl-1-methyl-7-oxo-3-propyl-1,7-dihydro-pyrazolo[4,3-d]pyrimidin-6-yl)-ethoxy]-phenylamino}-propionic acid (20B)

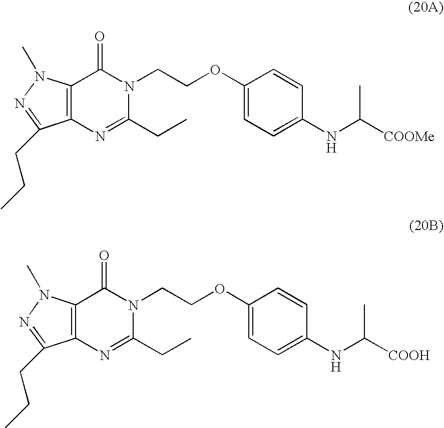

To a solution of 2-{4-[2-(5-ethyl-1-methyl-7-oxo-3-propyl-1,7-dihydro-pyrazolo[4,3-d]pyrimidin-6-yl)-ethoxy]-phenylamino}-propionic acid ethyl ester (150 mg, 0.329 mmol), obtained in example 19, in methanol (10 mL) sodium carbonate (175 mg, 1.65 mmol) in water (5 mL) was added and stirred at 20 to 40° C. for 24 hours. Methanol was evaporated under reduced pressure, water (25 mL) was added, extracted with ethyl acetate to remove non-polar impurities. Combined organic extracts were washed with brine, dried over anhydrous sodium sulfate and evaporated under reduced pressure. Crude product was triturated with 10% ethyl acetate in pet ether to afford title compound (20A).

Yield: White solid, 0.2 grams.

Melting Point: 100-104° C.

¹H NMR (CDCl₃, 200 MHz): δ 1.00 (t, J=7.3 Hz, 3H), 1.37 (t, J=7.6 Hz, 3H), 1.44 (d, J=7.3 Hz, 3H), 1.72-1.90 (m, 2H), 2.85 (t, J=7.6 Hz, 2H), 3.03 (q, J=7.3 Hz, 2H), 3.70 (s, 3H), 4.05 (q, J=7.1 Hz, 1H), 4.15-4.20 (m, 2H), 4.22 (s, 3H), 4.46 (t, J=5.4 Hz, 2H), 6.54 (d, J=8.8 Hz, 2H), 6.71 (d, J=8.8 Hz, 2H).

Mass (CI): m/z 442 (M⁺+1).

The aqueous layer was neutralized with 2N hydrochloric acid (pH 7.0), extracted with ethyl acetate twice. Combined organic extracts were washed with brine, dried over anhydrous sodium sulfate and evaporated under reduced pressure. Crude product was triturated with 50% ethyl acetate in pet ether, filtered off and dried to afford title compound as white solid (20B).

Yield: 70 mg, 50%.

Melting Point: 170-174° C.

¹H NMR (CDCl₃, 200 MHz): δ 0.98 (t, J=7.3 Hz, 3H), 1.36 (t, J=7.3 Hz, 3H), 1.49 (d, J=6.8 Hz, 3H), 1.72-1.90 (m, 2H), 2.84 (t, J=7.6 Hz, 2H), 3.02 (q, J=7.2 Hz, 2H), 3.94-4.08 (m, 1H), 4.12-4.28 (m, 5H), 4.46 (t, J=5.1 Hz, 2H), 6.59 (d, J=8.8 Hz, 2H), 6.72 (d, J=8.8 Hz, 2H).

Mass (CI): m/z 382 (M⁺-CO₂).

### EXAMPLE 21

### 2-Methyl-2-{4-[2-(2-methyl-4-oxo-4H-quinazolin-3-yl)-ethoxy]-phenylsulfanyl}-propionic acid ethyl ester

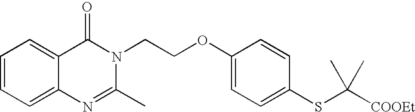

To a solution of ethyl 2-(4-hydroxyphenylsulfanyl)-2-methylpropanoate (0.4 grams, 1.67 mmol) in dimethylformamide (6 mL) potassium carbonate (690 mg, 5.0 mmol) was added and stirred for 30 minutes at 20 to 40° C. 3-(2-chloroethyl)-2-methyl-3,4-dihydro-4-quinazolinone (482 mg, 2.17 mmol), obtained in preparation 4, was added and the reaction mixture was stirred at 20 to 40° C. for 20 hours followed by heating at 60° C. for 20 hours. The reaction mixture was then diluted with ethyl acetate and washed with water, dried (sodium sulphate) and concentrated. The crude compound was then purified by column chromatography on 100-200 mesh silica gel using 95% ethyl acetate in hexane to yield pure title compound. Yield: 0.29 grams, 41%. Melting Point: 114-116° C.

¹H NMR (CDCl₃, 300 MHz): δ 1.20 (t, J=7.2 Hz, 3H), 1.42 (s, 6H), 2.81 (s, 3H), 4.08 (q, J=7.1 Hz, 2H), 4.34 (t, J=4.9 Hz, 2H), 4.53 (t, J=4.9 Hz, 2H), 6.79 (d, J=8.4 Hz, 2H), 7.35 (d, J=8.7 Hz, 2H), 7.43 (t, J=7.8 Hz, 1H), 7.62 (d, J=7.8 Hz, 1H), 7.73 (t, J=7.8 Hz, 1H), 8.24 (d, J=7.8 Hz, 1H). Mass (CI): m/z 427 (M⁺+1).

### EXAMPLE 22

### 2-Methyl-2-{4-[2-(2-methyl-4-oxo-4H-quinazolin-3-yl)-ethoxy]-phenylsulfanyl}-propionic acid

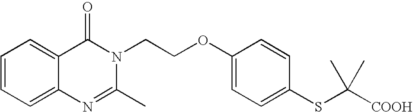

To a solution of 2-methyl-2-{4-[2-(2-methyl-4-oxo-4H-quinazolin-3-yl)-ethoxy]-phenylsulfanyl}-propionic acid ethyl ester (270 mg, 0.63 mmol), obtained in example 21, in tetrahydrofuran (5 mL) lithium hydroxide (53 mg, 1.27 mmol) in water (1 mL) was added. The reaction mixture was stirred at 20 to 40° C. for 48 hours followed by heating at 60° C. for 40 hours, the reaction mixture was then concentrated; water was added and extracted with ethyl acetate. Separated organic layer was discarded and the aqueous layer was acidified (pH ˜2) by using 2N hydrochloric acid solution at 0-5° C. The precipitated product was filtered, washed with cold water and dried to afford title compound as off white solid. Yield: 150 mg, 59.5%. Melting Point: 188-190° C.

¹H NMR (CDCl₃, 300 MHz): δ 1.45 (s, 6H), 2.79 (s, 3H), 4.34 (t, J=5.0 Hz, 2H), 4.52 (t, J=5.0 Hz, 2H), 6.79 (d, J=8.9 Hz, 2H), 7.39 (d, J=8.9 Hz, 2H), 7.44 (t, J=8.2 Hz, 1H), 7.65 (d, J=7.8 Hz, 1H), 7.73 (t, J=8.5 Hz, 1H), 8.23 (dd, J=1.2 & 8.1 Hz, 1H).

Mass (CI): m/z 399 (M⁺+1).

### EXAMPLE 23

### 2-{4-[3-(2-Ethyl-4-oxo-4H-quinazolin-3-yl)-propoxy]-phenylsulfanyl}-2-methyl-propionic acid ethyl ester

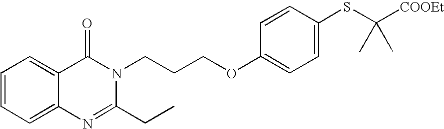

The title compound was prepared by following the same procedure as described in example 21 by using ethyl 2-(4-hydroxyphenylsulfanyl)-2-methylpropanoate (0.4 grams, 1.67 mmol) and 3-(3-chloropropyl)-2-ethyl-3,4-dihydro-4-quinazolinone (672 mg, 2.17 mmol), obtained in preparation 17, in the presence of potassium carbonate (0.69 grams, 5.0 mmol) in dimethylformamide (6 mL) for 72 hours. Yield: 0.53 grams, 70%.

¹H NMR (CDCl₃, 300 MHz): δ 1.24 (t, J=7.2 Hz, 3H), 1.41 (t, J=7.3 Hz, 3H), 1.46 (s, 6H), 2.20-2-35 (m, 2H), 2.91 (q, J=7.4 Hz, 2H), 4.02-4.19 (m, 4H), 4.32 (t, J=7.4 Hz, 2H), 6.82 (d, J=8.7 Hz, 2H), 7.38 (d, J=8.7 Hz, 2H), 7.43 (t, J=6.7 Hz, 1H), 7.60-7.78 (m, 1H), 7.6 (m, 1H), 8.24 (d, J=7.8 Hz, 1H). Mass (CI): m/z 455 (M⁺+1).

### EXAMPLE 24

### 2-{4-[3-(2-Ethyl-4-oxo-4H-quinazolin-3-yl)-propoxy]-phenylsulfanyl}-2-methyl-propionic acid

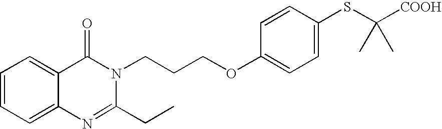

The title compound was prepared by following the same procedure as described in example 22 by using 2-{4-[3-(2-ethyl-4-oxo-4H-quinazolin-3-yl)-propoxy]-phenylsulfanyl}-2-methyl-propionic acid ethyl ester (0.52 grams, 1.15 mmol), obtained in example 23, in tetrahydrofuran-water (6 mL, 5:1) using lithium hydroxide (96 mg, 2.29 mmol) at 20 to 40° C. for 12 to 17 hours. Yield: 260 mg, 53.3% Melting Point 156-158° C.

¹H NMR (CDCl₃, 400 MHz): δ 1.40 (t, J=7.4 Hz, 3H), 1.49 (s, 6H), 2.22-2.28 (m, 2H), 2.90 (q, J=7.4 Hz, 2H), 4.09 (t, J=5.6 Hz, 2H), 4.32 (t, J=7.4 Hz, 2H), 6.82 (dd, J=2.1 & 6.7 Hz, 2H), 7.40-7.46 (m, 3H), 7.65 (dd, J=0.8 and 7.6 Hz, 1H), 7.69-7.75 (m, 1H), 8.23 (dd, J=1.6 & 8.0 Hz, 1H). Mass (CI): m/z 427 (M⁺+1).

### EXAMPLE 25

### 2-Methyl-2-{4-[3-(2-methyl-4-oxo-4H-quinazolin-3-yl)-propoxy]-phenylsulfanyl}-propionic acid ethyl ester

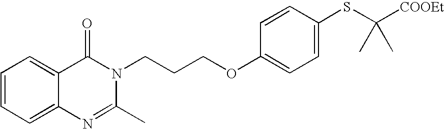

The title compound was prepared by following the same procedure as described in example 21 by using ethyl 2-(4-hydroxy-phenylsulfanyl)-2-methylpropanoate (0.4 grams, 1.67 mmol) and 3-(2-methyl-4-oxo-3,4-dihydro-3-quinazolinyl)propyl methane-sulfonate (493 mg, 2.17 mmol), obtained in preparation 43, in the presence of potassium carbonate (0.69 grams, 5.0 mmol) in dimethylformamide (6 mL) for 10 days. Yield: 0.64 grams, 87.3%. Melting Point: 162-164° C.

¹H NMR (CDCl₃, 300 MHz): δ 1.23 (t, J=7.0 Hz, 3H), 1.45 (s, 6H), 2.20-2.34 (m, 2H), 2.68 (s, 3H), 4.02-4.18 (m, 4H), 4.32 (t, J=7.5 Hz, 2H), 6.82 (d, J=8.7 Hz, 2H), 7.38 (d, J=8.4 Hz, 2H), 7.46 (t, J=7.3 Hz, 1H), 7.61 (d, J=8.1 Hz, 1H), 7.73 (t, J=7.6 Hz, 1H), 8.24 (d, J=7.8 Hz, 1H).

Mass (CI): m/z 441 (M⁺+1).

### EXAMPLE 26

### 2-Methyl-2-{4-[3-(2-methyl-4-oxo-4H-quinazolin-3-yl)-propoxy]-phenylsulfanyl}-propionic acid

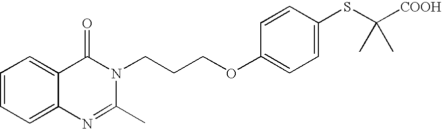

The title compound was prepared by following the same procedure as described in example 20 by treating 2-methyl-2-{4-[3-(2-methyl-4-oxo-4H-quinazolin-3-yl)-propoxy]-phenylsulfanyl}-propionic acid ethyl ester (600 mg, 1.36 mmol), obtained in example 25, in methanol-water (8 mL, 3:1) with sodium carbonate (723 mg, 6.82 mmol) for 12 days. Yield: 270 mg, 48%. Melting Point 144-146° C.

¹H NMR (CDCl₃, 400 MHz): δ 1.49 (s, 6H), 2.22-2-29 (m, 2H), 2.67 (s, 3H), 4.08 (t, J=5.6 Hz, 2H), 4.31 (t, J=7.4 Hz, 2H), 6.82 (dd, J=2.1 & 6.7 Hz, 2H), 7.41-7.47 (m, 3H), 7.64 (d, J=8.1 Hz, 1H), 7.69-7.75 (m, 1H), 8.23 (dd, J=1.3 & 8.1 Hz, 1H).

Mass (CI): m/z 413 (M⁺+1).

### EXAMPLE 27

### 2-{4-[3-(1-Ethyl-5-methyl-7-oxo-3-propyl-1,7-dihydro-pyrazolo[4,3-d]pyrimidin-6-yl)-propoxy]-phenylsulfanyl}-2-methyl-propionic acid ethylester

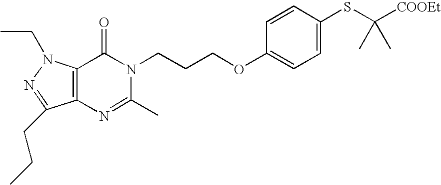

The title compound was prepared by following the same procedure as described in example 21 by using ethyl-2-(4-hydroxy-phenylsulfanyl)-2-methylpropanoate (235 mg, 0.98 mmol) and 6-(3-Bromopropyl)-1-ethyl-5-methyl-3-propyl-6,7-dihydro-1H-pyrazolo[4,3-d]-pyrimidin-7-one (0.4 grams, 1.18 mmol), obtained in preparation 38, in the presence of potassium carbonate (405 mg, 2.94 mmol) in dimethylformamide (5 mL) at 20 to 40° C. for 36 hours. Yield: 0.433 grams, 89%.

¹H NMR (CDCl₃, 200 MHz): δ 1.00 (t, J=7.3 Hz, 3H), 1.24 (t, J=7.3 Hz, 3H), 1.38-1.50 (m, 9H), 1.70-1.91 (m, 2H), 2.10-2.31 (m, 2H), 2.64 (s, 3H), 2.85 (t, J=7.6 Hz, 2H), 4.05-4.17 (m, 4H), 4.29 (t, J=7.3 Hz, 2H), 4.59 (q, J=7.3 Hz, 2H), 6.81 (d, J=9.0 Hz, 2H), 7.38 (d, J=8.4 Hz, 2H).

Mass (CI): m/z 501 (M⁺+1).

### EXAMPLE 28

### 2-{4-[3-(1-Ethyl-5-methyl-7-oxo-3-propyl-1,7-dihydro-pyrazolo[4,3-d]pyrimidin-6-yl)-propoxy]-phenylsulfanyl}-2-methyl-propionic acid

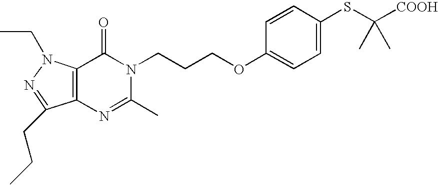

To a solution of 2-{4-[3-(1-ethyl-5-methyl-7-oxo-3-propyl-1,7-dihydro-pyrazolo[4,3-d]pyrimidin-6-yl)-propoxy]-phenylsulfanyl}-2-methyl-propionic acid ethylester (0.425 grams, 0.85 mmol), obtained in example 27, in ethanol (5 mL) powdered potassium hydroxide (0.286 grams, 5.1 mmol) was added and the reaction mixture was refluxed for 1 hour under nitrogen. The reaction mixture was cooled to 20 to 40° C. and the pH was adjusted to 2 using acetic acid. Acetic acid and ethanol were removed under reduced pressure and the solids were triturated with chloroform and the chloroform layer was concentrated. The crude mass was purified on silica gel (100-200 mesh) using 2% methanol in chloroform to afford the title compound as white solid.

Yield: 0.39 grams, 97%. Melting Point: 118-120° C.

¹H NMR (CDCl₃, 200 MHz): δ 0.99 (t, J=7.4 Hz, 3H), 1.44 (t, J=7.3 Hz, 3H), 1.48 (s, 6H), 1.76-1.86 (m, 2H), 2.20-2.28 (m, 2H), 2.64 (s, 3H), 2.85 (t, J=7.6 Hz, 2H), 4.09 (t, J=5.6 Hz, 2H), 4.29 (t, J=7.4 Hz, 2H), 4.58 (q, J=7.3 Hz, 2H), 6.81 (d, J=8.9 Hz, 2H), 7.43 (d, J=8.9 Hz, 2H). Mass (CI): m/z 473 (M⁺+1).

### EXAMPLE 29

### 2-{4-[3-(1,5-Diethyl-7-oxo-3-propyl-1,7-dihydro-pyrazolo[4,3-d]pyrimidin-6-yl)-propoxy]-phenylsulfanyl}-2-methyl-propionic acid ethyl ester

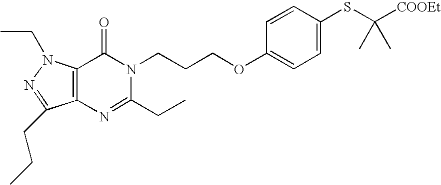

The title compound was prepared by following the same procedure as described in example 21 by using ethyl 2-(4-hydroxy-phenylsulfanyl)-2-methylpropanoate (185 mg, 0.77 mmol) and 6-(3-bromopropyl)-1,5-diethyl-3-propyl-6,7-dihydro-1H-pyrazolo[4,3-d]pyrimidin-7-one (0.328 grams, 0.92 mmol), obtained in preparation 37, in the presence of potassium carbonate (319 mg, 2.31 mmol) in dimethylformamide (5 mL) at 20 to 40° C. for 36 hours. Yield: 0.328 grams, 83%.

¹H NMR (CDCl₃, 200 MHz): δ 1.00 (t, J=7.3 Hz, 3H), 1.24 (t, J=7.0 Hz, 3H), 1.33 (t, J=7.3 Hz, 3H), 1.40-1.50 (m, 9H), 1.72, 1.92 (m, 2H), 2.14-2.29 (m, 2H), 2.80-2.96 (m, 4H), 4.05-4.18 (m, 4H), 4.30 (t, J=7.3 Hz, 2H), 4.60 (q, J=7.3 Hz, 2H), 6.79 (d, J=9.0 Hz, 2H), 7.38 (d, J=8.4 Hz, 2H).

Mass (CI): m/z 515 (M⁺+1).

### EXAMPLE 30

### 2-{4-[3-(1,5-Diethyl-7-oxo-3-propyl-1,7-dihydro-pyrazolo[4,3-d]pyrimidin-6-yl)-propoxy]-phenylsulfanyl}-2-methyl-propionic acid

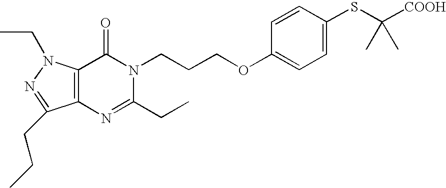

The title compound was prepared by following the same procedure as described in example 27, by refluxing 2-{4-[3-(1,5-diethyl-7-oxo-3-propyl-1,7-dihydro-pyrazolo[4,3-d]pyrimidin-6-yl)-propoxy]-phenylsulfanyl}-2-methyl-propionic acid ethyl ester (318 mg, 0.619 mmol), obtained in example 29, and powdered potassium hydroxide (208 mg, 3.7 mmol) in ethanol (5 mL) for 1 hour. Yield: 150 mg, 50%. Melting Point: 96-98° C.

¹H NMR (CDCl₃, 200 MHz): δ 1.00 (t, J=7.3 Hz, 3H), 1.36 (t, J=7.3 Hz, 3H), 1.44 (t, J=7.1 Hz, 3H), 1.49 (s, 6H), 1.78-1.87 (m, 2H), 2.18-2.28 (m, 2H), 2.81-2.91 (m, 4H), 4.09 (t, J=5.6 Hz, 2H), 4.29 (t, J=7.4 Hz, 2H), 4.57 (q, J=7.2 Hz, 2H), 6.82 (d, J=8.6 Hz, 2H), 7.43 (d, J=8.6 Hz, 2H). Mass (CI): m/z 487 (M⁺+1).

### EXAMPLE 31

### 2-{4-[2-(1,5-Dimethyl-7-oxo-3-propyl-1,7-dihydro-pyrazolo[4,3-d]pyrimidin-6-yl)-ethoxy]-phenylsulfanyl}-2-methyl-propionic acid ethyl ester

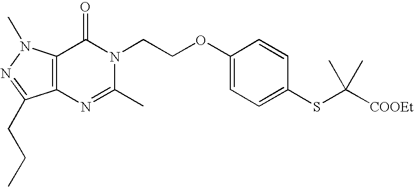

The title compound was obtained as solid following the same procedure as described in example 1 by heating ethyl 2-(4-hydroxyphenylsulfanyl)-2-methylpropanoate (658 mg, 2.74 mmol) and 6-(2-chloroethyl)-1,5-dimethyl-3-propyl-6,7-dihydro-1H-pyrazolo[4,3-d]pyrimidin-7-one (700 mg, 2.61 mmol) in the presence of potassium carbonate (1.08 grams, 7.83 mmol) and tetrabutylammonium bromide (42 mg, 0.13 mmol) in toluene (7 mL) at 90° C. for 48 hours. Yield: 881 mg, 71%. Melting Point: 106-108° C.

¹H NMR (CDCl₃, 200 MHz): δ 0.99 (t, J=7.4 Hz, 3H), 1.22 (t, J=7.1 Hz, 3H), 1.42 (s, 6H), 1.70-1.89 (m, 2H), 2.76 (s, 3H), 2.83 (t, J=7.6 Hz, 2H), 4.09 (q, J=7.2 Hz, 2H), 4.22 (s, 3H), 4.30 (t, J=5.0 Hz, 2H), 4.49 (t, J=5.0 Hz, 2H), 6.8 (d, J=8.6 Hz, 2H), 7.32 (d, J=8.6 Hz, 2H). Mass (CI): m/z 473 (M⁺+1).

### EXAMPLE 32

### 2-{4-[2-(1,5-Dimethyl-7-oxo-3-propyl-1,7-dihydro-pyrazolo[4,3-d]pyrimidin-6-yl)-ethoxy]-phenylsulfanyl}-2-methyl-propionic acid

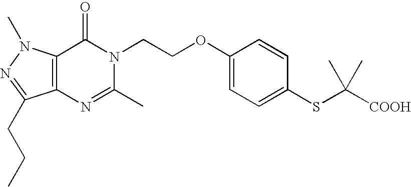

To a solution of 2-{4-[2-(1,5-dimethyl-7-oxo-3-propyl-1,7-dihydro-pyrazolo[4,3-d]pyrimidin-6-yl)-ethoxy]-phenylsulfanyl}-2-methyl-propionic acid ethyl ester (200 mg, 0.39 mmol), obtained in example 31, in methanol (6 mL) a saturated solution of sodium carbonate (208 mg, 1.95 mmol) in water was added and the reaction mixture was stirred at 20 to 40° C. for 10 days. The reaction mixture was then concentrated, diluted with water and extracted with ethyl acetate. The organic layer was discarded and the aqueous layer was acidified (pH ˜2) by 2N hydrochloric acid at 0-5° C. The separated solid was filtered and washed with cold water to afford the title compound. Yield: 165 mg, 85%.

Melting Point: 182-184° C.

¹H NMR (CDCl₃, 200 MHz): δ 0.99 (t, J=7.4 Hz, 3H), 1.45 (s, 6H), 1.70-188 (m, 2H), 2.76 (s, 3H), 2.83 (t, J=7.6 Hz, 2H), 4.22 (s, 3H), 4.24-4.56 (m, 4H), 6.8 (d, J=8.6 Hz, 2H), 7.4 (d, J=8.6 Hz, 2H). Mass (CI): m/z 445 (M⁺+1).

### EXAMPLE 33

### 2-{4-[2-(2-Ethyl-4-oxo-4H-quinazolin-3-yl)-ethoxy]-phenylsulfanyl}-2-methyl-propionic acid ethyl ester

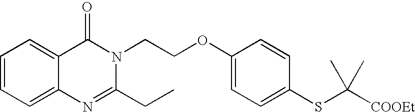

The title compound was obtained following the same procedure as described in example 21 by using ethyl 2-(4-hydroxy-phenylsulfanyl)-2-methylpropanoate (400 mg, 1.67 mmol and 3-(2-chloroethyl)-2-ethyl-3,4-dihydro-4-quinazolinone (512 mg, 2.17 mmol), obtained in preparation 7, in the presence of potassium carbonate (690 mg, 5.0 mmol) in dimethylformamide (8 mL) at 60° C. for 48 hours. Yield: 130 mg, 17.7%. Melting Point: 120-122° C.

¹H NMR (CDCl₃, 300 MHz): δ 1.20 (t, J=7.1 Hz, 3H), 1.40-1.50 (m, 3H), 1.42 (s, 6H), 3.09 (q, J=7.4 Hz, 2H), 4.08 (q, J=7.0 Hz, 2H), 4.33 (t, J=5.0 Hz, 2H), 4.54 (t, J=5.0 Hz, 2H), 6.79 (d, J=8.7 Hz, 2H), 7.34 (, J=8.7 Hz, 2H), 7.43 (t, J=8.0 Hz, 1H), 7.62-7.80 (m, 2H), 8.24 (d, J=7.8 Hz, 1H). Mass (CI): m/z 441 (M⁺+1).

### EXAMPLE 34

### 2-{4-[2-(2-Ethyl-4-oxo-4H-quinazolin-3-yl)-ethoxy]-phenylsulfanyl}-2-methyl-propionic acid

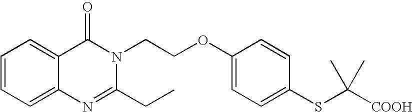

To a solution of Ethyl 2-{4-[2-(2-ethyl-4-oxo-4H-quinazolin-3-yl)-ethoxy]-phenylsulfanyl}-2-methyl-propionic acid ethyl ester (115 mg, 0.26 mmol), obtained in example 33, in dimethylsulphoxide, (3 mL) Esteraze (10 mg, 410 unit) was added followed by the addition of 0.01M potasiumphosphate (pH=8.0, 18 mL). The reaction mixture was stirred at 35° C. for 72 hours. The reaction mixture was cooled to 0° C. and the pH was adjusted to 6. The aqueous layer was extracted with ethyl acetate and the organic layer was washed successively with water and brine, dried over sodium sulphate and concentrated. Column purification on 100-200 mesh silica gel using 70% ethyl acetate in pet ether afforded pure title compound. Yield: 35 mg, 32.5%. Melting Point: 148-150° C.

¹H NMR (CDCl₃, 400 MHz): δ 1.42 (t, J=7.4 Hz, 3H), 1.45 (s, 6H), 3.09 (q, J=7.4 Hz, 2H), 4.33 (t, J=5.2 Hz, 2H), 4.53 (t, J=5.2 Hz, 2H), 6.79 (dd, J=2.0 & 6.8 Hz, 2H), 7.39 (dd, J=2.1 & 6.7 Hz, 2H), 7.41-7.46 (m, 1H), 7.66 (dd, J=0.8 & 8.1 Hz, 1H), 7.70-7.74 (m, 1H), 8.23 (d, J=1.1 & 8.1 Hz, 1H). Mass (CI): m/z 413 (M⁺+1).

### EXAMPLE 35

### 2-{4-[3-(1,5-Dimethyl-7-oxo-3-propyl-1,7-dihydro-pyrazolo[4,3-d]pyrimidin-6-yl)-propoxy]-phenylsulfanyl}-2-methyl-propionic acid ethyl ester

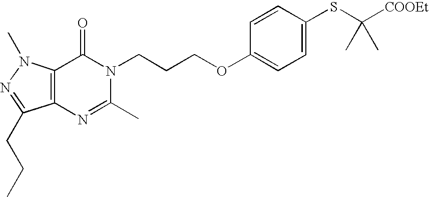

The title compound was prepared as white solid following the same procedure as described in example 1, by heating ethyl 2-(4-hydroxyphenylsulfanyl)-2-methylpropanoate (283 mg, 1.18 mmol) and 6-(3-bromopropyl)-1,5-dimethyl-3-propyl-6,7-dihydro-1H-pyrazolo-[4,3-d]pyrimidin-7-one (350 mg, 1.07 mmol) in the presence of potassium carbonate (490 mg, 3.53 mmol) and tetrabutylammonium bromide (18 mg, 0.056 mmol) in toluene (9 mL) at 90° C. for 40 hours. Yield: 470 mg, 90%. Melting Point: 52-54° C.

¹H NMR (CDCl₃, 200 MHz): δ 1.0 (t, J=7.3 Hz, 3H), 1.23 (t, J=7.1 Hz, 3H), 1.45 (s, 6H), 1.71-1.89 (m, 2H), 2.17-2.30 (m, 2H), 2.63 (s, 3H), 2.84 (t, J=7.6 Hz, 2H), 4.04-4.17 (m, 4H), 4.21 (s, 3H), 4.29 (t, J=7.5 Hz, 2H), 6.81 (d, J=8.8 Hz, 2H), 7.38 (d, J=8.8 Hz, 2H). Mass (CI): m/z 487 (M⁺+1).

### EXAMPLE 36

### 2-{4-[3-(1,5-Dimethyl-7-oxo-3-propyl-1,7-dihydro-pyrazolo[4,3-d]pyrimidin-6-yl)-propoxy]-phenylsulfanyl}-2-methyl-propionic acid

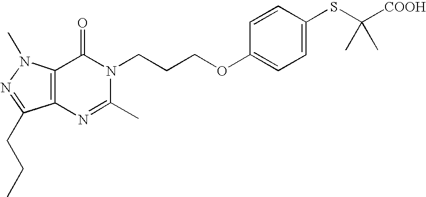

The title compound was prepared as a white solid following the same procedure as described in example 32, by treating 2-{4-[3-(1,5-dimethyl-7-oxo-3-propyl-1,7-dihydro-pyrazolo[4,3-d]pyrimidin-6-yl)-propoxy]-phenylsulfanyl}-2-methyl-propionic acid ethyl ester (200 mg, 0.39 mmol), obtained in example 35, in a mixture of methanol (6 mL) and a saturated solution of sodium carbonate (208 mg 1.95 mmol) for 12 days at 20 to 40° C. Yield: 165 mg, 85%. Melting Point: 140-142° C.

¹H NMR (CDCl₃, 200 MHz): δ 1.0 (t, J=7.3 Hz, 3H), 1.48 (s, 6H), 1.70-1.88 (m, 2H), 2.16-2.30 (m, 2H), 2.63 (s, 3H), 2.84 (t, J=7.8 Hz, 2H), 4.08 (t, J=5.8 Hz, 2H), 4.20 (s, 3H), 4.29 (t, J=7.3 Hz, 2H), 6.80 (d, J=8.3 Hz, 2H), 7.43 (d, J=8.3 Hz, 2H).

Mass (CI): m/z 415 (M⁺-CO₂+1).

### EXAMPLE 37

### 2-{4-[3-(5-Ethyl-1-methyl-7-oxo-3-propyl-1,7-dihydro-pyrazolo[4,3-d]pyrimidin-6-yl)-propylamino]-phenylsulfanyl}-2-methyl-propionic acid ethyl ester

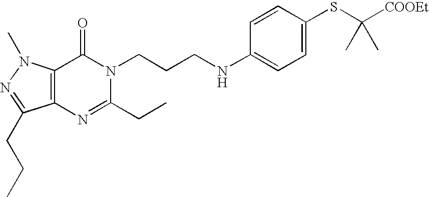

The title compound was prepared by following the same procedure as described in example 1, by heating ethyl-2-(4-aminophenylsulfanyl)-2-methylpropanoate (248 mg, 1.03 mmol) and 6-(3-bromopropyl)-5-ethyl-1-methyl-3-propyl-6,7-dihydro-1H-pyrazolo[4,3-d]pyrimidin-7-one (355 mg, 1.03 mmol) in the presence of potassium carbonate (430 mg, 3.1 mmol) and tetrabutylammonium bromide (17 mg, 0.053 mmol) in toluene (9 mL) at 90° C. for 72 hours. Yield: 170 mg, 32%.

¹H NMR (CDCl₃, 200 MHz): δ 0.99 (t, J=7.3 Hz, 3H), 1.24 (t, J=7.1 Hz, 3H), 1.34 (t, J=7.3 Hz, 3H), 1.44 (s, 6H), 1.73-1.91 (m, 2H), 1.96-2.08 (m, 2H), 2.74-2.88 (m, 4H), 3.23 (t, J=6.3 Hz, 2H), 4.06-4.19 (m, 4H), 4.24 (s, 3H), 6.56 (d, J=8.3 Hz, 2H), 7.27 (d, J=8.3 Hz, 2H). Mass (CI): m/z 500 (M⁺+1).

### EXAMPLE 38

### 2-{4-[3-(5-Ethyl-1-methyl-7-oxo-3-propyl-1,7-dihydro-pyrazolo[4,3-d]pyrimidin-6-yl)-propylamino]-phenylsulfanyl}-2-methyl-propionic acid

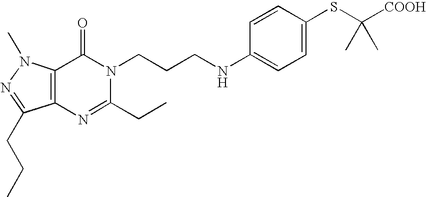

The title was prepared as a white solid following the same procedure as described in example 32 by treating the compound from 2-{4-[3-(5-ethyl-1-methyl-7-oxo-3-propyl-1,7-dihydro-pyrazolo[4,3-d]pyrimidin-6-yl)-propylamino]-phenylsulfanyl}-2-methyl-propionic acid ethyl ester (125 mg, 0.25 mmol), obtained in example 37, in a mixture of methanol (5 mL) and a saturated solution of sodium carbonate (133 mg, 1.25 mmol) for 9 days at 20 to 40° C. Yield: 85 mg 72%.

### EXAMPLE 39

### 2-{4-[3-(5-Ethyl-1-methyl-7-oxo-3-propyl-1,7-dihydro-pyrazolo[4,3-d]pyrimidin-6-yl)-propoxy]-phenylsulfanyl}-2-methyl-propionic acid ethyl ester

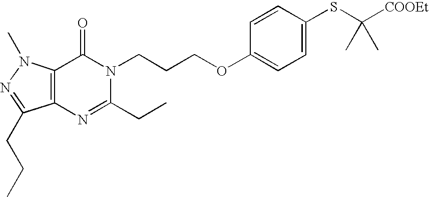

The title was prepared as white solid following the same procedure as described in example 1, by heating ethyl 2-(4-hydroxyphenylsulfanyl)-2-methylpropanoate (245 mg, 1.02 mmol) and 6-(3-bromopropyl)-1,5-dimethyl-3-propyl-6,7-dihydro-1H-pyrazolo-[4,3-d]pyrimidin-7-one (350 mg, 1.02 mmol) in the presence of potassium carbonate (430 mg, 3.06 mmol) and tetrabutylammonium bromide (17 mg, 0.05 mmol) in toluene (10 mL) at 90° C. for 48 hours. Yield: 400 mg, 78%. Melting Point: 52-54° C.

¹H NMR (CDCl₃, 200 MHz): δ 1.0 (t, J=7.4 Hz, 3H), 1.24 (t, J=7.1 Hz, 3H), 1.36 (t, J=7.4 Hz, 3H), 1.58 (s, 6H), 1.74-1.92 (m, 2H), 2.16-2.26 (m, 2H), 2.81-2.94 (m, 4H), 4.04-4.18 (m, 4H), 4.21 (s, 3H), 4.29 (t, J=7.5 Hz, 2H), 6.81 (d, J=8.6 Hz, 2H), 7.38 (d, J=8.6 Hz, 2H). Mass (CI): m/z 501 (M⁺+1).

### EXAMPLE 40

### 2-{4-[3-(5-Ethyl-1-methyl-7-oxo-3-propyl-1,7-dihydro-pyrazolo[4,3-d]pyrimidin-6-yl)-propoxy]-phenylsulfanyl}-2-methyl-propionic acid

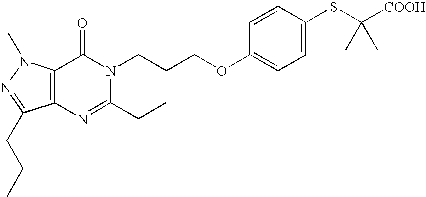

The title compound was prepared as colorless solid following the same procedure as described in example 32, by treating 2-{4-[3-(5-ethyl-1-methyl-7-oxo-3-propyl-1,7-dihydro-pyrazolo[4,3-d]pyrimidin-6-yl)-propoxy]-phenylsulfanyl}-2-methyl-propionic acid ethyl ester (270 mg, 0.54 mmol), obtained in example 39, in a mixture of methanol (5 mL) and saturated solution of sodium carbonate (287 mg, 2.7 mmol) for 10 days at 20 to 40° C. Yield: 200 mg, 78%. Melting Point: 115-117° C.

¹H NMR (CDCl₃, 200 MHz): δ 1.0 (t, J=7.2 Hz, 3H), 1.36 (t, J=7.4 Hz, 3H), 1.49 (s, 6H), 1.74-1.92 (m, 2H), 1.94-2.08 (m, 2H), 2.82-3.12 (m, 4H), 4.09 (t, J=5.8 Hz, 2H), 4.20 (s, 3H), 4.23 (t, J=7.3 Hz, 2H), 6.81 (d, J=8.6 Hz, 2H), 7.44 (d, J=8.6 Hz, 2H).

Mass (CI): m/z 473 (M⁺+1).

### EXAMPLE 41

### 2-{4-[2-(5-Ethyl-1-methyl-7-oxo-3-propyl-1,7-dihydro-pyrazolo[4,3-d]pyrimidin-6-yl)-ethylamino]-phenylsulfanyl}-2-methyl-propionic acid ethyl ester

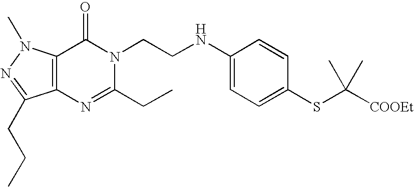

The title compound was obtained as yellow solid following the same procedure as described in the example 1, by heating ethyl 2-(4-aminophenylsulfanyl)-2-methylpropanoate (423 mg, 1.77 mmol) and 6-(2-chloroethyl)-5-ethyl-1-methyl-3-propyl-6,7-dihydro-1H-pyrazolo[4,3-d]pyrimidin-7-one (500 mg, 1.77 mmol) in the presence of potassium carbonate (736 mg, 5.32 mmol) and tetrabutylammonium bromide (29 mg, 0.089 mmol) in toluene (10 mL) at 90° C. for 72 hours. Yield: 165 mg, 19%. Melting Point: 105-107° C.

¹H NMR (CDCl₃, 200 MHz): δ 0.99 (t, J=7.2 Hz, 3H), 1.23 (t, J=7.1 Hz, 3H), 1.34 (t, J=7.2 Hz, 3H), 1.43 (s, 6H), 1.74-1.91 (m, 2H), 2.73-2.88 (m, 4H), 3.46-3.57 (m, 2H), 4.11 (q, J=7.1 Hz, 2H), 4.25 (s, 3H), 4.4 (t, J=6.1 Hz, 2H), 6.56 (d, J=8.6 Hz, 2H), 7.27 (d, J=8.6 Hz, 2H). Mass (CI): m/z 486 (M⁺+1).

### EXAMPLE 42

### 2-{4-[2-(5-Ethyl-1-methyl-7-oxo-3-propyl-1,7-dihydro-pyrazolo[4,3-d]pyrimidin-6-yl)-ethylamino]-phenylsulfanyl}-2-methyl-propionic acid

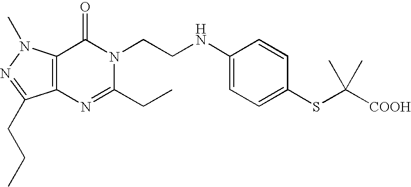

The title compound was prepared as off white solid following the same procedure as described in example 20, by treating 2-{4-[2-(5-ethyl-1-methyl-7-oxo-3-propyl-1,7-dihydro-pyrazolo[4,3-d]pyrimidin-6-yl)-ethylamino]-phenylsulfanyl}-2-methyl-propionic acid ethyl ester (60 mg, 0.128 mmol), obtained in example 41, with sodium carbonate (68 mg, 0.64 mmol) in methanol-water (4 mL, 1:1) at 20 to 40° C. for 13 days.

Yield: 20 mg, 36%.

Melting Point 187-189° C.

¹H NMR (CDCl₃, 400 MHz): δ 0.99 (t, J=7.4 Hz, 3H), 1.33 (t, J=7.4 Hz, 3H), 1.42 (s, 6H), 1.75-1.85 (m, 2H), 2.79-2.87 (m, 4H), 3.51 (t, J=6.6 Hz, 2H), 4.24 (s, 3H), 4.32 (t, J=6.6 Hz, 2H), 6.61 (d, J=8.6 Hz, 2H), 7.44 (d, J=8.9 Hz, 2H).

Mass (CI): m/z 458 (M⁺+1).

### EXAMPLE 43

### 2-{4-[2-(5-Ethyl-1-methyl-7-oxo-3-propyl-1,7-dihydro-pyrazolo[4,3-d]pyrimidin-6-yl)-ethoxy]-phenylsulfanyl}-2-methyl-propionic acid ethyl ester

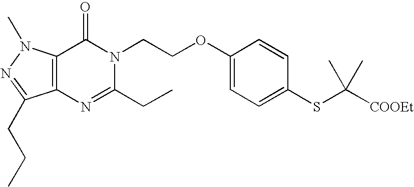

The title compound was prepared by following the same procedure as described in example 1 by heating ethyl-2-(4-hydroxyphenylsulfanyl)-2-methylpropanoate (298 mg, 1.24 mmol) and 6-(2-chloroethyl)-5-ethyl-1-methyl-3-propyl-6,7-dihydro-1H-pyrazolo-[4,3-d]pyrimidin-7-one (350 mg, 1.24 mmol) in the presence of potassium carbonate (515 mg, 3.72 mmol) and tetrabutylammonium bromide (20 mg, 0.062 mmol) in toluene (10 mL) at 90° C. for 60 hours

Yield: 480 mg, 80%.

¹H NMR (CDCl₃, 200 MHz): δ 0.99 (t, J=7.4 Hz, 3H), 1.22 (t, J=7.1 Hz, 3H), 1.38 (t, J=7.2 Hz, 3H), 1.42 (s, 6H), 1.72-1.92 (m, 2H), 2.85 (t, J=7.5 Hz, 2H), 3.03 (q, J=7.4 Hz, 2H), 4.09 (q, J=7.2 Hz, 2H), 4.22 (s, 3H), 4.28 (t, J=5.2 Hz, 2H), 4.50 (t, J=5.2 Hz, 2H), 6.78 (d, J=8.6 Hz, 2H), 7.35 (d, J=8.6 Hz, 2H).

Mass (CI): m/z 487 (M⁺+1).

### EXAMPLE 44

### 2-{4-[2-(5-Ethyl-1-methyl-7-oxo-3-propyl-1,7-dihydro-pyrazolo[4,3-d]pyrimidin-6-yl)-ethoxy]-phenylsulfanyl}-2-methyl-propionic acid

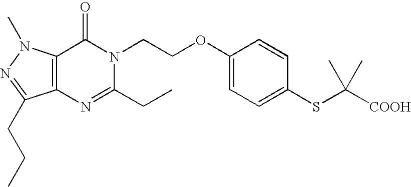

The title compound was prepared as white solid following the same procedure as described in example 32, by treating 2-{4-[2-(5-ethyl-1-methyl-7-oxo-3-propyl-1,7-dihydro-pyrazolo[4,3-d]pyrimidin-6-yl)-ethoxy]-phenylsulfanyl}-2-methyl-propionic acid ethyl ester (480 mg, 0.99 mmol), obtained in example 43, in a mixture of methanol (5 mL) and a saturated solution of sodium carbonate (524 mg, 4.94 mmol) for 9 days at 20 to 40° C.

Yield: 220 mg, 48%.

Melting Point: 104-106° C.

¹H NMR (CDCl₃, 200 MHz): δ 0.99 (t, J=7.2 Hz, 3H), 1.38 (t, J=7.4 Hz, 3H), 1.45 (s, 6H), 1.74-1.91 (m, 2H), 2.85 (t, J=7.3 Hz, 2H), 3.04 (q, J=7.2 Hz, 2H), 4.22 (s, 3H), 4.29 (t, J=7.3 Hz, 2H), 4.50 (t, J=4.9 Hz, 2H), 6.80 (d, J=8.6 Hz, 2H), 7.40 (d, J=8.6 Hz, 2H).

Mass (CI): m/z 459 (M⁺+1).

### EXAMPLE 45

### 2-{4-[3-(1,5-Dimethyl-7-oxo-3-propyl-1,7-dihydro-pyrazolo[4,3-d]pyrimidin-6-yl)-propylamino]-phenylsulfanyl}-2-methyl-propionic acid ethyl ester

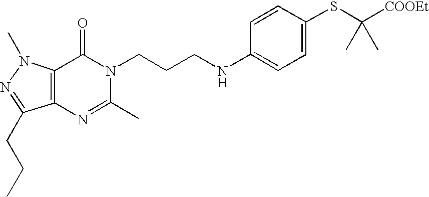

The title compound was prepared by following the same procedure as described in example 1, by heating ethyl 2-(4-aminophenylsulfanyl)-2-methylpropanoate (282 mg, 1.17 mmol) and 6-(3-bromopropyl)-1,5-dimethyl-3-propyl-6,7-dihydro-1H-pyrazolo[4,3-d]pyrimidin-7-one (350 mg, 1.07 mmol) in the presence of potassium carbonate (490 mg, 3.52 mmol) and tetrabutylammonium bromide (18 mg, 0.054 mmol) in toluene (10 mL) at 90° C. for 72 hours.

Yield: 230 mg, 40%

¹H NMR (CDCl₃, 200 MHz): δ 0.99 (t, J=7.3 Hz, 3H), 1.23 (t, J=7.0 Hz, 3H), 1.44 (s, 6H), 1.71-1.88 (m, 2H), 1.96-2.10 (m, 2H), 2.60 (s, 3H), 2.83 (t, J=7.5 Hz, 2H), 3.23 (t, J=6.4 Hz, 2H), 4.06-4.22 (m, 4H), 4.23 (s, 3H), 6.56 (d, J=8.8 Hz, 2H), 7.26 (d, J=8.3 Hz, 2H).

Mass (CI): m/z 486 (M⁺+1).

### EXAMPLE 46

### 2-{4-[3-(1,5-Dimethyl-7-oxo-3-propyl-1,7-dihydro-pyrazolo[4,3-d]pyrimidin-6-yl)-propylamino]-phenylsulfanyl}-2-methyl-propionic acid

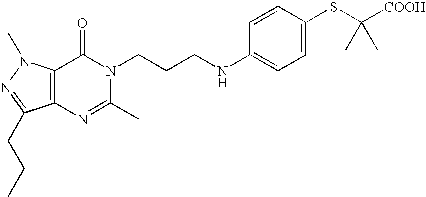

The title compound was prepared as a white solid following the same procedure as described in example 32, by treating 2-{4-[3-(1,5-dimethyl-7-oxo-3-propyl-1,7-dihydro-pyrazolo[4,3-d]pyrimidin-6-yl)-propylamino]-phenylsulfanyl}-2-methyl-propionic acid ethyl ester (190 mg, 0.39 mmol), obtained in example 45, in a mixture of methanol (5 mL) and saturated solution of sodium carbonate (208 mg, 1.96 mmol) for 10 days at 20 to 40° C.

Yield: 130 mg, 72%.

Melting Point: 134-136° C.

¹H NMR (CDCl₃, 200 MHz): δ 0.99 (t, J=7.3 Hz, 3H), 1.47 (s, 6H), 1.72-1.90 (m, 2H), 1.94-2.12 (m, 2H), 2.58 (s, 3H), 2.84 (t, J=7.8 Hz, 2H), 3.24 (t, J=6.3 Hz, 2H), 4.19 (t, J=7.3 Hz, 2H), 4.23 (s, 3H), 6.56 (d, J=8.8 Hz, 2H), 7.31 (d, J=8.3 Hz, 2H).

### EXAMPLE 47

### 2-{4-[2-(1,5-Diethyl-7-oxo-3-propyl-1,7-dihydro-pyrazolo[4,3-d]pyrimidin-6-yl)-ethylamino]-phenylsulfanyl}-2-methyl-propionic acid ethyl ester

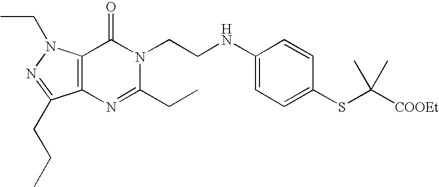

The title compound was prepared by following the same procedure as described in the example 1, by heating ethyl 2-(4-aminophenylsulfanyl)-2-methylpropanoate (1.0 gram, 4.37 mmol) and 6-(2-chloroethyl)-1,5-diethyl-3-propyl-6,7-dihydro-1H-pyrazolo[4,3-d]pyrimidin-7-one (700 mg, 2.36 mmol), obtained in preparation 47, in the presence of potassium carbonate (1.21 grams 8.75 mmol) and tetrabutylammonium bromide (470 mg, 1.46 mmol) in toluene (10 mL) at 120° C. for 24 hours.

Yield: 820 mg, 70%.

Melting Point: 85-87° C.

¹H NMR (CDCl₃, 400 MHz): δ 0.99 (t, J=7.4 Hz, 3H), 1.23 (t, J=7.2 Hz, 3H), 1.34 (t, J=7.3 Hz, 3H), 1.43 (s, 6H), 1.48 (t, J=7.3 Hz, 3H), 1.76-1.88 (m, 2H), 2.79 (q, J=7.3 Hz, 2H), 2.85 (t, J=7.6 Hz, 2H), 3.51 (q, J=5.9 Hz, 2H), 4.11 (q, J=7.1 Hz, 2H), 4.35 (t, J=6.3 Hz, 2H), 4.62 (q, J=7.3 Hz, 2H), 6.56 (d, J=8.9 Hz, 2H), 7.27 (d, J=8.6 Hz, 2H).

Mass (CI): m/z 499 (M⁺+1).

### EXAMPLE 48

### 2-{4-[2-(1,5-Diethyl-7-oxo-3-propyl-1,7-dihydro-pyrazolo[4,3-d]pyrimidin-6-yl)-ethylamino]-phenylsulfanyl}-2-methyl-propionic acid

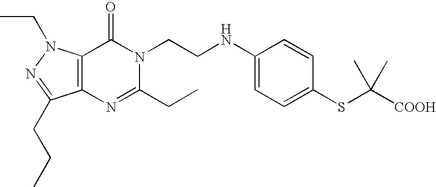

The title compound was prepared as off white solid following the same procedure as described in example 20 by treating 2-{4-[2-(1,5-Diethyl-7-oxo-3-propyl-1,7-dihydro-pyrazolo[4,3-d]pyrimidin-6-yl)-ethylamino]-phenylsulfanyl}-2-methyl-propionic acid ethyl ester (325 mg, 0.65 mmol), obtained in example 47, with sodium carbonate (345 mg, 3.25 mmol) in methanol-water (6 mL, 1:1) at 20 to 40° C. for 10 days.

Yield: 175 mg, 57%.

Melting Point: 143-145° C.

¹H NMR (CDCl₃, 400 MHz): δ 0.98 (t, J=7.4 Hz, 3H), 1.33 (t, J=7.4 Hz, 3H), 1.45 (s, 6H), 1.47 (t, J=7.3 Hz, 3H), 1.77-1.85 (m, 2H), 2.78 (q, J=7.3 Hz, 2H), 2.85 (t, J=7.5 Hz, 2H), 3.51 (t, J=6.2 Hz, 2H), 4.34 (t, J=6.9 Hz, 2H), 4.61 (q, J=7.2 Hz, 2H), 6.56 (dd, J=1.9 & 6.7 Hz, 2H), 7.31 (dd, J=2.1 & 6.7 Hz, 2H).

Mass (CI): m/z 472 (M⁺+1).

### EXAMPLE 49

### 2-{4-[2-(1,5-Diethyl-7-oxo-3-propyl-1,7-dihydro-pyrazolo[4,3-d]pyrimidin-6-yl)-ethoxy]-phenylsulfanyl}-2-methyl-propionic acid ethyl ester

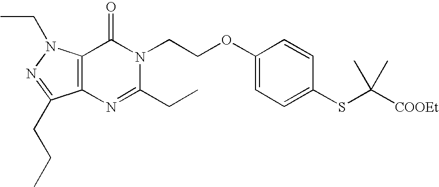

The title compound was obtained as pale yellow liquid following the same procedure as described in example 21 by using ethyl 2-(4-hydroxyphenylsulfanyl)-2-methylpropanoate (200 mg, 0.83 mmol) and 6-(2-chloroethyl)-1,5-diethyl-3-propyl-6,7-dihydro-1H-pyrazolo[4,3-d]pyrimidin-7-one (296 mg, 1.0 mmol), obtained in preparation 47, in the presence of potassium carbonate (345 mg, 2.49 mmol) in dimethylformamide (5 mL) for 36 hours.

Yield: 0.2 grams, 48%.

¹H NMR (CDCl₃, 400 MHz): δ 0.99 (t, J=7.4 Hz, 3H), 1.22 (t, J=7.1 Hz, 3H), 1.39 (t, J=7.4 Hz, 3H), 1.43 (s, 6H), 1.47 (t, J=7.3 Hz, 3H), 1.77-1.88 (m, 2H), 2.86 (t, J=7.7 Hz, 2H), 3.02 (q, J=7.3 Hz, 2H), 4.09 (q, J=7.2 Hz, 2H), 4.29 (t, J=5.4 Hz, 2H), 4.51 (t, J=5.4 Hz, 2H), 4.60 (q, J=7.2 Hz, 2H), 6.79 (dd, J=2.1 & 6.7 Hz, 2H), 7.36 (dd, J=2.1 &6.7 Hz, 2H).

Mass (CI): m/z 501 (M⁺+1).

### EXAMPLE 50

### 2-{4-[2-(1,5-Diethyl-7-oxo-3-propyl-1,7-dihydro-pyrazolo[4,3-d]pyrimidin-6-yl)-ethoxy]-phenylsulfanyl}-2-methyl-propionic acid

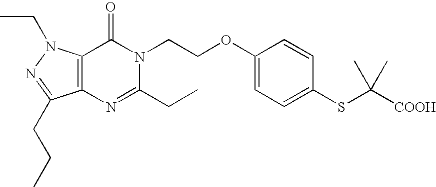

The title compound was prepared by following the same procedure as described in example 27, by refluxing 2-{4-[2-(1,5-diethyl-7-oxo-3-propyl-1,7-dihydro-pyrazolo[4,3-d]pyrimidin-6-yl)-ethoxy]-phenylsulfanyl}-2-methyl-propionic acid ethyl ester (190 mg, 0.38 mmol), obtained in example 49, and powdered potassium hydroxide (128 mg, 2.28 mmol) in ethanol (5 mL) for 24 hours.

Yield: 138 mg, 77%.

Melting Point: 123-125° C.

¹H NMR (CDCl₃, 400 MHz): δ 0.99 (t, J=7.4 Hz, 3H), 1.38 (t, J=7.2 Hz, 3H), 1.45 (s, 6H), 1.46 (t, J=7.0 Hz, 3H), 1.78-1.87 (m, 2H), 2.86 (t, J=7.7 Hz, 2H), 3.02 (q, J=7.3 Hz, 2H), 4.30 (t, J=5.2 Hz, 2H), 4.50 (t, J=5.2 Hz, 2H), 4.59 (q, J=7.3 Hz, 2H), 6.80 (d, J=8.9 Hz, 2H), 7.40 (d, J=8.6 Hz, 2H).

Mass (CI): m/z 473 (M⁺+1).

### EXAMPLE 51

### 2-{4-[2-(1-Ethyl-5-methyl-7-oxo-3-propyl-1,7-dihydro-pyrazolo[4,3-d]pyrimidin-6-yl)-ethoxy]-phenylsulfanyl}-2-methyl-propionic acid ethyl ester

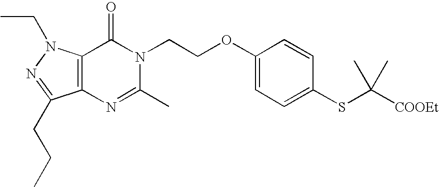

The title compound was obtained as solid following the same procedure as described in example 21, by heating ethyl 2-(4-hydroxyphenylsulfanyl)-2-methylpropanoate (200 mg, 0.83 mmol) and 6-(2-chloroethyl)-1-ethyl-5-methyl-3-propyl-6,7-dihydro-1H-pyrazolo[4,3-d]-pyrimidin-7-one (259 mg, 0.92 mmol), obtained in preparation 45, in the presence of potassium carbonate (380 mg, 2.75 mmol) in dimethylformamide (5 mL) at 60° C. for 24 hours.

Yield: 118 mg, 29%

Melting Point: 85-87° C.

¹H NMR (CDCl₃, 400 MHz): δ 0.99 (t, J=7.4 Hz, 3H), 1.22 (t, J=7.2 Hz, 3H), 1.43 (s, 6H), 1.46 (t, J=7.1 Hz, 3H), 1.77-1.86 (m, 2H), 2.76 (s, 3H), 2.85 (t, J=7.7 Hz, 2H), 4.10 (q, J=7.2 Hz, 2H), 4.31 (t, J=5.2 Hz, 2H), 4.50 (t, J=5.1 Hz, 2H), 4.60 (q, J=7.2 Hz, 2H), 6.80 (dd, J=2.1, 6.7 Hz, 2H), 7.35 (dd, J=2.2, 6.8 Hz, 2H).

Mass (CI): m/z 487 (M⁺+1).

### EXAMPLE 52

### 2-{4-[2-(1-Ethyl-5-methyl-7-oxo-3-propyl-1,7-dihydro-pyrazolo[4,3-d]pyrimidin-6-yl)-ethoxy]-phenylsulfanyl}-2-methyl-propionic acid

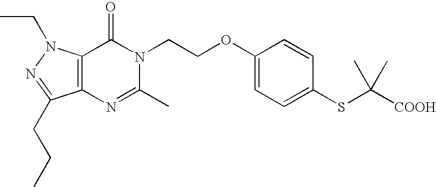

The title compound was obtained as off white solid following the same procedure as described in example 27 by refluxing 2-{4-[2-(1-ethyl-5-methyl-7-oxo-3-propyl-1,7-dihydro-pyrazolo[4,3-d]pyrimidin-6-yl)-ethoxy]-phenylsulfanyl}-2-methyl-propionic acid ethyl ester (105 mg, 0.216 mmol), obtained in example 51, and powdered potassium hydroxide (73 mg, 1.3 mmol) in ethanol (2 mL) for 24 hours.

Yield: 22 mg, 22%.

Melting Point: 178-180° C.

¹H NMR (CDCl₃, 400 MHz): δ 0.99 (t, J=7.4 Hz, 3H), 1.45 (s, 6H), 1.46 (t, J=7.2 Hz, 3H), 1.76-1.84 (m, 2H), 2.75 (s, 3H), 2.84 (t, J=7.7 Hz, 2H), 4.32 (t, J=5.1 Hz, 2H), 4.50 (t, J=5.1 Hz, 2H), 4.59 (q, J=7.3 Hz, 2H), 6.81 (dd, J=2.0 & 6.9 Hz, 2H), 7.40 (dd, J=2.1 & 6.7 Hz, 2H).

Mass (CI): m/z 459 (M⁺+1).

### EXAMPLE 53

### 2-{4-[2-(1-Ethyl-5-methyl-7-oxo-3-propyl-1,7-dihydro-pyrazolo[4,3-d]pyrimidin-6-yl)-ethoxy]-phenylsulfanyl}-2-methyl-butyric acid ethyl ester

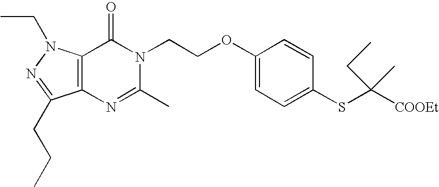

The title compound was obtained as pale yellow liquid following the same procedure as described in example 21 by heating ethyl 2-(4-hydroxyphenylsulfanyl)-2-methylbutanoate (200 mg, 0.79 mmol) and 6-(2-chloroethyl)-1-ethyl-5-methyl-3-propyl-6,7-dihydro-1H-pyrazolo[4,3-d]-pyrimidin-7-one (245 mg, 0.87 mmol), obtained in preparation 45, in the presence of potassium carbonate (326 mg, 2.36 mmol) in dimethylformamide (5 mL) at 60° C. for 48 hours.

Yield: 160 mg, 41%.

¹H NMR (CDCl₃, 400 MHz): δ 0.93 (t, J=7.4 Hz, 3H), 1.0 (t, J=7.4 Hz, 3H), 1.22 (t, J=7.1 Hz, 3H), 1.34 (s, 3H), 1.47 (t, J=7.3 Hz, 3H), 1.59-1.70 (m, 1H), 1.75-1.86 (m, 2H), 1.88-1.94 (m, 1H), 2.76 (s, 3H), 2.85 (t, J=7.7 Hz, 2H), 4.06-4.15 (m, 2H), 4.31 (t, J=5.1 Hz, 2H), 4.50 (t, J=5.1 Hz, 2H), 4.60 (q, J=7.2 Hz, 2H), 6.80 (d, J=8.9 Hz, 2H), 7.34 (d, J=8.9 Hz, 2H).

Mass (CI): m/z 501 (M⁺+1).

### EXAMPLE 54

### 2-{4-[2-(1-Ethyl-5-methyl-7-oxo-3-propyl-1,7-dihydro-pyrazolo[4,3-d]pyrimidin-6-yl)-ethoxy]-phenylsulfanyl}-2-methyl-butyric acid

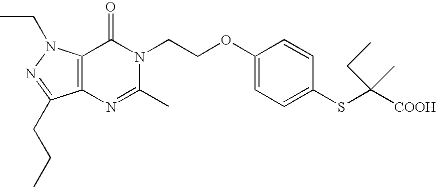

The title compound was obtained as off white solid following the same procedure as described in example 27 by refluxing 2-{4-[2-(1-ethyl-5-methyl-7-oxo-3-propyl-1,7-dihydro-pyrazolo[4,3-d]pyrimidin-6-yl)-ethoxy]-phenylsulfanyl}-2-methyl-butyric acid ethyl ester (150 mg, 0.3 mmol), obtained in example 53, and powdered potassium hydroxide (101 mg, 1.8 mmol) in ethanol (2 mL) for 48 hours.

Yield: 40 mg, 28%.

Melting Point: 155-157° C.

¹H NMR (CDCl₃, 400 MHz): δ 0.99 (t, J=7.4 Hz, 6H), 1.34 (s, 3H), 1.46 (t, J=7.3 Hz, 3H), 1.59-1.70 (m, 1H), 1.73-1.86 (m, 2H), 1.88-1.98 (m, 1H), 2.75 (s, 3H), 2.85 (t, J=7.7 Hz, 2H), 4.30 (t, J=5.1 Hz, 2H), 4.49 (t, J=5.1 Hz, 2H), 4.59 (q, J=7.2 Hz, 2H), 6.80 (dd, J=2.1 & 6.7 Hz, 2H), 7.40 (dd, J=2.1 & 6.7 Hz, 2H).

Mass (ES): m/z 473 (M⁺+1).

### EXAMPLE 55

### 2-Methyl-2-{4-[3-(2-methyl-4-oxo-4H-quinazolin-3-yl)-propoxy]-phenylsulfanyl}-butyric acid ethyl ester

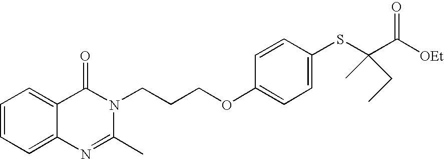

The title compound was obtained as off white solid following the same procedure as described in the example 1 by heating ethyl-2-(4-hydroxyphenylsulfanyl)-2-methylbutanoate (350 mg, 1.38 mmol) and 3-(2-methyl-4-oxo-3,4-dihydro-3-quinazolinyl)propyl methanesulfonate (530 mg, 1.79 mmol), obtained in preparation 43, in the presence of potassium carbonate (570 mg, 4.13 mmol) and tetrabutylammonium bromide (89 mg, 0.275 mmol) in toluene (10 mL) at 130-140° C. for 20 hours.

Yield: 0.45 grams, 71.9%.

Melting Point: 164-166° C.

¹H NMR (CDCl₃, 200 MHz): δ 0.95 (t, J=7.1 Hz, 3H), 1.23 (t, J=7.1 Hz, 3H), 1.37 (s, 3H), 1.58-1.78 (m, 1H), 1.82-2.01 (m, 1H), 2.18-2.37 (m, 2H), 2.68 (s, 3H), 4.00-4.21 (m, 4H), 4.32 (t, J=7.2 Hz, 2H), 6.82 (d, J=7.8 Hz, 2H), 7.37 (d, J=8.1 Hz, 2H), 7.44 (t, J=7.6 Hz, 1H), 7.61 (d, J=8.1 Hz, 1H), 7.73 (t, J=7.5 Hz, 1H), 8.24 (d, J=8.0 Hz, 1H).

Mass (CI): m/z 455 (M⁺+1).

### EXAMPLE 56

### 2-Methyl-2-{4-[3-(2-methyl-4-oxo-4H-quinazolin-3-yl)-propoxy]-phenylsulfanyl}-butyric acid

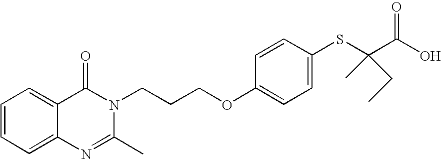

The title compound was obtained as off white solid following the same procedure as described in example 27 by refluxing 2-methyl-2-{4-[3-(2-methyl-4-oxo-4H-quinazolin-3-yl)-propoxy]-phenylsulfanyl}-butyric acid ethyl ester (230 mg, 0.51 mmol), obtained in example 55, and powdered potassium hydroxide (170 mg, 3.04 mmol) in ethanol (5 mL) for 24 hours.

Yield: 160 mg, 74%.

Mp: 130-132° C.

¹H NMR (CDCl₃, 400 MHz): δ 1.01 (t, J=7.4 Hz, 3H), 1.39 (s, 3H), 1.68-1.76 (m, 1H), 1.91-2.00 (m, 1H), 2.22-2.31 (m, 2H), 2.67 (s, 3H), 4.08 (t, J=5.6 Hz, 2H), 4.32 (t, J=7.4 Hz, 2H), 6.81 (dd, J=1.9 & 6.7 Hz, 2H), 7.42 (dd, J=2.1 & 6.7 Hz, 2H), 7.44 (t, J=7.0 Hz, 1H), 7.63 (d, J=7.5 Hz, 1H), 7.70-7.75 (m, 1H), 8.23 (dd, J=1.3 & 8.1 Hz, 1H).

Mass (CI): m/z 427 (M⁺+1).

### EXAMPLE 57

### 2-Methyl-2-{4-[2-(1-methyl-7-oxo-3-propyl-1,7-dihydro-pyrazolo[4,3-d]pyrimidin-6-yl)-ethoxy]-phenoxy}-propionic acid ethyl ester

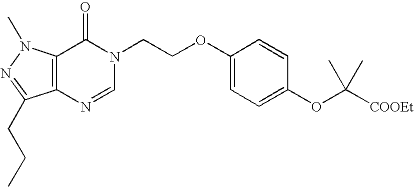

To a solution of 2-(4-hydroxy-phenoxy)-2-methyl-propionic acid ethyl ester (200 mg, 0.89 mmol), obtained in preparation 50, in dimethylformamide (10 mL) potassium carbonate (370 mg, 2.67 mmol) was added and stirred for 30 minutes. 6-(2-bromoethyl)-1-methyl-3-propyl-6,7-dihydro-1H-pyrazolo[4,3-d]pyrimidin-7-one (1.33 grams, 4.46 mmol), obtained in preparation 41, in DMF (2 mL) was then added dropwise at 0° C. The reaction mixture was heated at 60° C. for 48 hours, the reaction mixture was diluted with ethyl acetate and the organic layer was washed with water and brine, dried (sodium sulphate) and evaporated to dryness. Column purification with 40% ethyl acetate in pet ether afforded the title compound.

Yield: 141 mg, 36%).

¹H NMR (CDCl₃, 200 MHz): δ 7.93 (s, 1H), 6.80 & 6.71 (2d, J=9.1 Hz, 4H), 4.36 (t, J=4.5 Hz, 2H), 4.26 (s, 3H), 4.29-4.11 (m, 2H), 2.84 (t, J=7.6 Hz, 2H), 1.89-1.68 (m, 2H), 1.50 (s, 6H), 1.25 (t, J=7.1 Hz, 3H), 0.98 (J=7.4 Hz, 3H).

Mass (CI): m/z 443 (M⁺+1).

IR (cm⁻¹) (KBr): 2960, 1734, 1690, 1505.

### EXAMPLE 58

### 2-Methyl-2-{4-[2-(1-methyl-7-oxo-3-propyl-1,7-dihydro-pyrazolo[4,3-d]pyrimidin-6-yl)-ethoxy]-phenoxy}-propionic acid

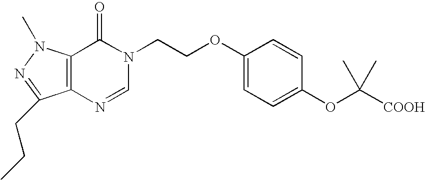

The title compound was prepared by following the same procedure as described in example 57 by treating 2-methyl-2-{4-[2-(1-methyl-7-oxo-3-propyl-1,7-dihydro-pyrazolo[4,3-d]pyrimidin-6-yl)-ethoxy]-phenoxy}-propionic acid ethyl ester (138 mg, 0.31 mmol), obtained in example 57, with sodium carbonate (165 mg, 1.56 mmol) in methanol-water (6 mL, 1:1) at 20 to 40° C. for 12 hours.

Yield: 104 mg, 80%.

Melting Point: 144-146° C.

¹H NMR (CDCl₃, 200 MHz): δ 8.10 (s, 1H), 6.90 & 6.75 (2d, J=9.1 Hz, 4H), 4.36 (t, J=4.5 Hz, 2H), 4.44-4.38 (m, 2H), 4.30-4.18 (m, 5H), 2.84 (t, J=7.6 Hz, 2H), 1.85-1.65 (m, 2H), 1.53 (s, 6H), 0.98 (J=7.3 Hz, 3H).

Mass (CI): m/z 415 (M⁺+1).

IR (cm⁻¹) (KBr): 3432, 2933, 1723, 1688, 1584, 1513.

### EXAMPLE 59

### 2-{4-[2-(1,5-Dimethyl-7-oxo-3-propyl-1,7-dihydro-pyrazolo[4,3-d]pyrimidin-6-yl)-ethoxy]-phenoxy}-2-methyl-propionic acid ethyl ester

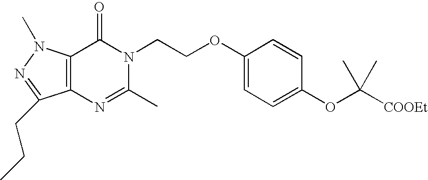

To a solution of 6-(2-chloroethyl)-1,5-dimethyl-3-propyl-6,7-dihydro-1H-pyrazolo[4,3-d]pyrimidin-7-one (500 mg, 1.87 mmol), 2-(4-hydroxy-phenoxy)-2-methyl-propionic acid ethyl ester (460 mg, 2.02 mmol)), obtained in preparation 50, and potassium carbonate (850 mg, 6.15 mmol) in toluene (9 mL) tetrabutylammonium bromide (33 mg, 0.11 mmol) was added and heated at 90° C. for 48 hours. The reaction mixture was diluted with ethyl acetate and the organic layer was washed with water and brine, dried (sodium sulphate) and evaporated to dryness. Column purification with 20% ethyl acetate in pet ether afforded the pure title compound.

Yield: 660 mg, 70%.

Melting Point: 98-100° C.

¹HNMR (CDCl₃, 200 MHz): δ 6.81 (d, J=9.1 Hz, 2H), 6.72 (d, J=9.1 Hz, 2H), 4.47 (t, J=5.1 Hz, 2H), 4.27-4.16 (m, 7H), 2.83 (t, J=7.8 Hz, 2H), 2.75 (s, 3H), 1.89-1.71 (m, 2H), 1.51 (s, 6H), 1.26 (t, J=7.1 Hz, 3H), 0.99 (t, J=7.4 Hz, 3H).

Mass (CI): m/z 457 (M⁺+1).

IR (cm⁻¹) (Neat): 2960, 1735, 1694, 1510.

### EXAMPLE 60

### 2-{4-[2-(1,5-Dimethyl-7-oxo-3-propyl-1,7-dihydro-pyrazolo[4,3-d]pyrimidin-6-yl)-ethoxy]-phenoxy}-2-methyl-propionic acid

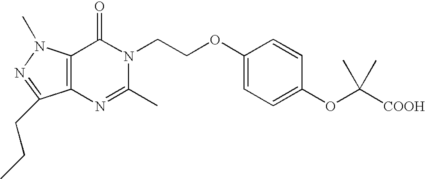

The title compound was prepared by following the same procedure as described in example 59 by treating 2-{4-[2-(1,5-dimethyl-7-oxo-3-propyl-1,7-dihydro-pyrazolo[4,3-d]pyrimidin-6-yl)-ethoxy]-phenoxy}-2-methyl-propionic acid ethyl ester (200 mg, 0.44 mmol), obtained in example 59, in a mixture of methanol (5 mL) and saturated solution of sodium carbonate (233 mg, 2.19 mmol) for 24 hours at 20 to 40° C.

Yield: (160 mg, 85%).

Melting Point: 146-148° C.

¹H NMR (CDCl₃, 200 MHz): δ 6.90 (d, J=8.8 Hz, 2H), 6.75 (d, J=9.1 Hz, 2H), 4.48 (t, J=4.8 Hz, 2H), 4.26 (t, J=4.8H, 2H), 4.22 (s, 3H), 2.83 (t, J=7.5 Hz, 2H), 2.77 (s, 3H), 1.89-1.71 (m, 2H), 1.52 (s, 6H), 0.98 (t, J=7.2 Hz, 3H).

Mass (CI): m/z 429 (M⁺+1).

IR (cm⁻¹) (KBr): 3436, 2935, 1730, 1701, 1506.

### EXAMPLE 61

### 2-{4-[2-(1,5-Dimethyl-7-oxo-3-propyl-1,7-dihydro-pyrazolo[4,3-d]pyrimidin-6-yl)-ethylamino]-phenoxy}-2-methyl-propionic acid ethyl ester

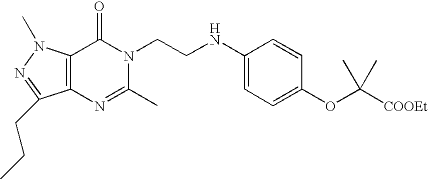

To a solution of 6-(2-chloroethyl)-1,5-dimethyl-3-propyl-6,7-dihydro-1H-pyrazolo[4,3-d]pyrimidin-7-one (500 mg, 1.87 mmol), 2-(4-amino-phenoxy)-2-methyl-propionic acid ethyl ester (461 mg, 2.05 mmol)), obtained in preparation 51, and potassium carbonate (850 mg, 6.15 mmol) in toluene (9 mL) tetrabutylammonium bromide (33 mg, 0.11 mmol) was added and heated at 100° C. for 72 hours. The reaction mixture was diluted with ethyl acetate and the organic layer was washed with water and brine, dried over sodium sulphate and evaporated to dryness. Column purification with 20% ethyl acetate in petroleum ether to give title compound.

Yield: 160 mg.

¹H NMR (CDCl₃, 200 MHz): δ 6.79 (d, J=9.3 Hz, 2H), 6.54 (d, J=8.8 Hz, 2H), 4.30-4.20 (m, 7H), 3.57-3.44 (m, 2H), 2.84-2.74 (m, 2H), 2.56 (s, 3H), 1.82-1.64 (m, 2H), 1.50 (s, 6H), 1.29 (t, J=7.1 Hz, 3H), 0.99 (t, J=7.3 Hz, 3H).

Mass (CI): m/z 456 (M⁺+1).

IR (cm⁻¹) (KBr): 2961, 2873, 1739, 1689, 1574, 1512.

### EXAMPLE 62

### 2-{4-[2-(1,5-Dimethyl-7-oxo-3-propyl-1,7-dihydro-pyrazolo[4,3-d]pyrimidin-6-yl)-ethylamino]-phenoxy}-2-methyl-propionic acid

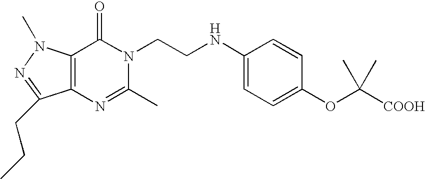

The title compound was prepared by following the same procedure as described in example 61 by treating 2-{4-[2-(1,5-dimethyl-7-oxo-3-propyl-1,7-dihydro-pyrazolo[4,3-d]pyrimidin-6-yl)-ethylamino]-phenoxy}-2-methyl-propionic acid ethyl ester (150 mg, 0.33 mmol), obtained in example 61, in methanol-water (1:1 ratio, 10 mL) using sodium carbonate (175 mg, 1.65 mmol) for 18 hours.

Yield: 120 mg, 85%.

Melting Point: 88-90° C.

¹H NMR (CDCl₃, 200 MHz): δ 6.82 (d, J=8.6 Hz, 2H), 6.58 (d, J=8.6 Hz, 2H), 4.31 (t, J=6.2 Hz, 2H), 4.23 (s, 3H), 3.50 (t, J=6.0 Hz, 2H), 2.82 (t, J=7.5 Hz, 2H), 2.57 (s, 3H), 1.84-1.68 (m, 2H), 1.48 (s, 6H), 0.97 (t, J=7.2 Hz, 3H).

Mass (CI): m/z 428 (M⁺+1).

IR (cm⁻¹) (KBr): 3390, 2927, 1700, 1690, 1577, 1516.

### EXAMPLE 63

### 2-Methyl-2-{4-[3-(1-methyl-7-oxo-3-propyl-1,7-dihydro-pyrazolo[4,3-d]pyrimidin-6-yl)-propoxy]-phenoxy}-propionic acid ethyl ester

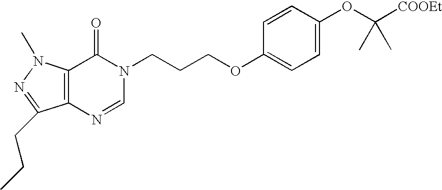

To a solution of 6-(3-bromopropyl)-1-methyl-3-propyl-6,7-dihydro-1H-pyrazolo[4,3-d]-pyrimidin-7-one (250 mg, 0.8 mmol), obtained in preparation 42, 2-(4-hydroxy-phenoxy)-2-methyl-propionic acid ethyl ester (196 mg, 0.88 mmol), obtained in preparation 50, and potassium carbonate (362 mg, 2.63 mmol) in toluene (10 mL) tetrabutylammonium bromide (13 mg, 0.04 mmol) was added and heated at 90° C. for 48 hours. The reaction mixture was diluted with ethyl acetate and the organic layer was washed with water and brine, dried over sodium sulphate and evaporated to dryness. Column purification with 60% ethyl acetate in petroleum ether afforded title compound.

Yield: 328 mg, 90%.

¹H NMR (CDCl₃, 200 MHz): δ 7.8 (s, 1H), 6.83 (d, J=9.3 Hz, 2H), 6.73 (d, J=8.8 Hz, 2H), 4.30-4.19 (m, 2H), 4.25 (s, 3H), 3.96 (t, J=5.9 Hz, 2H), 2.85 (t, J=7.8 Hz, 2H), 2.35-2.17 (m, 2H), 1.85-1.73 (m, 2H), 1.54 (s, 3H), 1.29 (t, J=7.1 Hz, 3H), 0.99 (t, J=7.3 Hz, 3H).

Mass (CI): m/z 457 (M⁺+1).

IR (cm⁻¹) (KBr): 2930, 1734, 1689, 1582, 1505.

### EXAMPLE 64

### 2-Methyl-2-{4-[3-(1-methyl-7-oxo-3-propyl-1,7-dihydro-pyrazolo[4,3-d]pyrimidin-6-yl)-propoxy]-phenoxy}-propionic acid

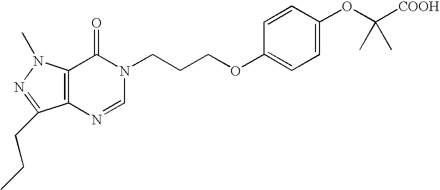

The title compound was prepared by following the same procedure as described in example 63 by treating 2-methyl-2-{4-[3-(1-methyl-7-oxo-3-propyl-1,7-dihydro-pyrazolo[4,3-d]pyrimidin-6-yl)-propoxy]-phenoxy}-propionic acid ethyl ester (328 mg, 0.72 mmol), obtained in example 63, in methanol-water (1:1 ratio, 10 mL) using sodium carbonate (381 mg, 3.59 mmol) for 12 hours. Methanol was evaporated and the residue was diluted with water. Aqueous layer was cooled to 0° C. and acidified with 2N hydrochloric acid upto pH 2. The aqueous layer was extracted with ethyl acetate. The organic layer was washed with water, dried over sodiumsulphate and evaporated to dryness to give the pure compound

Yield: 256 mg, 83%.

Melting Point: 136-138° C.

¹H NMR (CDCl₃, 200 MHz): δ 7.88 (s, 1H), 6.88 (d, J=9.3 Hz, 2H), 6.75 (d, J=8.9 Hz, 2H), 4.23-4.19 (m, 5H), 3.97 (t, J=5.5 Hz, 2H), 2.83 (t, J=7.5 Hz, 2H), 2.35-2.17 (m, 2H), 1.82-1.70 (m, 2H), 1.55 (s, 3H), 0.97 (t, J=7.2 Hz, 3H).

Mass (CI): m/z 429 (M⁺+1).

IR (cm⁻¹) (KBr): 3427, 2956, 1710, 1694, 1592, 1505.

### EXAMPLE 65

### 2-{4-[3-(1,5-Dimethyl-7-oxo-3-propyl-1,7-dihydro-pyrazolo[4,3-d]pyrimidin-6-yl)-propoxy]-phenoxy}-2-methyl-propionic acid ethyl ester

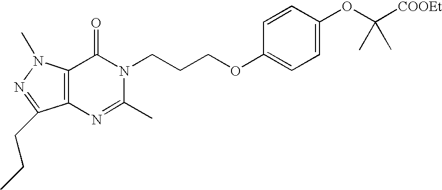

The title compound was prepared by following the same procedure as described in example 63, by heating 2-(4-hydroxy-phenoxy)-2-methyl-propionic acid ethyl ester (378 mg, 1.68 mmol)), obtained in preparation 50, and 6-(3-bromopropyl)-1,5-dimethyl-3-propyl-6,7-dihydro-1H-pyrazolo[4,3-d]pyrimidin-7-one (500 mg, 1.53 mmol) in the presence of potassium carbonate (700 mg, 5.06 mmol) and tetrabutylammonium bromide (27 mg, 0.08 mmol) in toluene (9 mL) at 90° C. for 48 hours.

¹HNMR (CDCl₃, 200 MHz): δ 6.84 (d, J=9.1 Hz, 2H), 6.74 (d, J=9.1 Hz, 2H), 4.31-4.21 (m, 7H), 4.01 (t, J=5.5 Hz, 2H), 2.84 (t, J=7.6 Hz, 2H), 2.63 (s, 3H), 2.24-2.14 (m, 2H), 1.84-1.70 (m, 2H), 1.54 (s, 6H), 1.28 (t, J=7.1 Hz, 3H), 1.00 (t, J=7.4 Hz, 3H).

MS (CI): m/z 471 (M⁺+1)

IR (cm⁻¹) (Neat): 2932, 1735, 1691, 1575, 1506.

### EXAMPLE 66

### 2-{4-[3-(1,5-Dimethyl-7-oxo-3-propyl-1,7-dihydro-pyrazolo[4,3-d]pyrimidin-6-yl)-propoxy]-phenoxy}-2-methyl-propionic acid

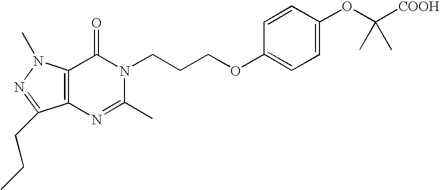

The title compound was prepared by following the same procedure as described in example 64 by treating 2-{4-[3-(1,5-dimethyl-7-oxo-3-propyl-1,7-dihydro-pyrazolo[4,3-d]pyrimidin-6-yl)-propoxy]-phenoxy}-2-methyl-propionic acid ethyl ester (328 mg, 0.72 mmol), obtained in example 65, in methanol (4 mL) using sodium carbonate (243 mg, 2.29 mmol) at 20 to 40° C. for 48 hours.

Yield: 180 mg, 90%.

Melting Point: 158-160° C.

¹H NMR (CDCl₃, 200 MHz): δ 6.91 (d, J=9.1 Hz, 2H), 6.77 (d, J=9.1 Hz, 2H), 4.29 (t, J=7.4 Hz, 2H), 4.20 (s, 3H), 4.04 (t, J=5.5 Hz, 2H), 2.84 (t, J=7.6 Hz, 2H), 2.64 (s, 3H), 2.28-2.14 (m, 2H), 1.90-1.70 (m, 2H), 1.54 (s, 6H), 1.00 (t, J=7.4 Hz, 3H).

Mass (CI): m/z 443 (M⁺+1).

IR (cm⁻¹) (KBr): 3433, 2960, 1695, 1577, 1506.

### EXAMPLE 67

### 2-{4-[3-(1,5-Dimethyl-7-oxo-3-propyl-1,7-dihydro-pyrazolo[4,3-d]pyrimidin-6-yl)-propylamino]-phenoxy}-2-methyl-propionic acid ethyl ester

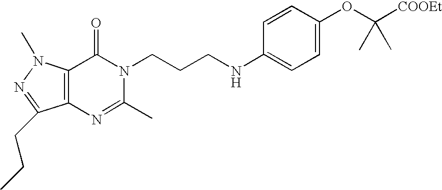

The title compound was prepared by following the same procedure as described in example 63, by heating 2-(4-amino-phenoxy)-2-methyl-propionic acid ethyl ester (375 mg, 1.68 mmol), obtained in preparation 51, and 6-(3-bromopropyl)-1,5-dimethyl-3-propyl-6,7-dihydro-1H-pyrazolo[4,3-d]pyrimidin-7-one (500 mg, 1.53 mmol) in the presence of potassium carbonate (698 mg, 5.04 mmol) and tetrabutylammonium bromide (25 mg, 0.08 mmol) in toluene (9 mL) at 90° C. for 48 hours.

¹H NMR (CDCl₃, 200 MHz): δ 6.75 (d, J=8.6 Hz, 2H), 6.50 (d, J=8.8 Hz, 2H), 4.26-4.04 (m, 7H), 3.15 (t, J=6.3 Hz, 2H), 2.80 (t, J=7.6 Hz, 2H), 2.54 (s, 3H), 2.05-1.90 (m, 2H), 1.84-1.68 (m, 2H), 1.47 (s, 6H), 1.25 (t, J=7.0 Hz, 3H), 0.95 (t, J=7.2 Hz, 3H).

Mass (CI): m/z 470 (M⁺+1).

IR (cm⁻¹) (KBr): 2961, 2873, 1733, 1681, 1573, 1512.

### EXAMPLE 68

### 2-{4-[3-(1,5-Dimethyl-7-oxo-3-propyl-1,7-dihydro-pyrazolo[4,3-d]pyrimidin-6-yl)-propylamino]-phenoxy}-2-methyl-propionic acid

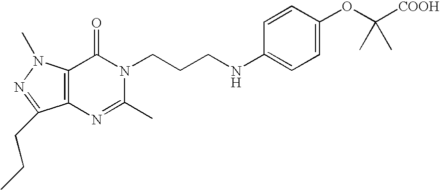

The title compound was prepared by following the same procedure as described in example 64 by treating 2-{4-[3-(1,5-dimethyl-7-oxo-3-propyl-1,7-dihydro-pyrazolo[4,3-d]pyrimidin-6-yl)-propylamino]-phenoxy}-2-methyl-propionic acid ethyl ester (400 mg, 0.85 mmol), obtained in example 67, in methanol (6 mL) using sodium carbonate (452 mg, 4.26 mmol) at 20 to 40° C. for 96 hours.

Yield: 180 mg, 90%.

Melting Point: 104-106° C.

¹H NMR (CDCl₃, 200 MHz): δ 6.83 (d, J=8.6 Hz, 2H), 6.58 (d, J=8.8 Hz, 2H), 4.23-4.16 (m, 5H), 3.21 (t, J=6.3 Hz, 2H), 2.83 (t, J=7.5 Hz, 2H), 2.59 (s, 3H), 2.10-1.96 (m, 2H), 1.76-1.68 (m, 2H), 1.51 (s, 6H), 0.99 (t, J=7.2 Hz, 3H).

Mass (CI): m/z 424 (M⁺+1).

IR (cm⁻¹) (KBr): 3400, 2930, 1710, 1690, 1574, 1512.

### EXAMPLE 69

### 2-{4-[2-(5-Ethyl-1-methyl-7-oxo-3-propyl-1,7-dihydro-pyrazolo[4,3-d]pyrimidin-6-yl)-ethoxy]-phenoxy}-2-methyl-propionic acid ethyl ester

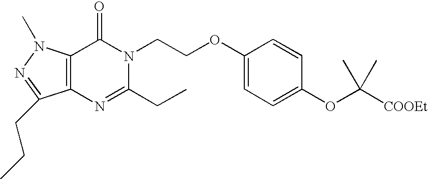

The title compound was obtained as solid following the same procedure as described in the example 63, by heating 6-(2-chloroethyl)-5-ethyl-1-methyl-3-propyl-6,7-dihydro-1H-pyrazolo[4,3-d]pyrimidin-7-one (350 mg, 1.3 mmol) and 2-(4-hydroxy-phenoxy)-2-methyl-propionic acid ethyl ester (320 mg, 1.43 mmol)), obtained in preparation 50, in the presence of potassium carbonate (590 mg, 4.28 mmol) and tetrabutylammonium bromide (23 mg, 0.065 mmol) in toluene (8 mL) at 90° C. for 48 hours.

Yield: 250 mg, 38%.

Melting Point: 66-68° C.

¹H NMR (CDCl₃, 200 MHz): δ 6.80 (d, J=9.2 Hz, 2H), 6.71 (d, J=8.8 Hz, 2H), 4.48 (t, J=5.4 Hz, 2H), 4.27-4.17 (m, 7H), 3.06 (q, J=7.3 Hz, 2H), 2.85 (t, J=7.5 Hz, 2H), 1.92-1.74 (m, 2H), 1.50 (s, 6H), 1.37 (t, J=7.3 Hz, 3H), 1.26 (t, J=7.0 Hz, 3H), 0.99 (t, J=7.6 Hz, 3H).

Mass (CI): m/z 471 (M⁺+1).

IR (cm⁻¹) (KBr): 2961, 1731, 1692, 1506.

### EXAMPLE 70

### 2-{4-[2-(5-Ethyl-1-methyl-7-oxo-3-propyl-1,7-dihydro-pyrazolo[4,3-d]pyrimidin-6-yl)-ethoxy]-phenoxy}-2-methyl-propionic acid

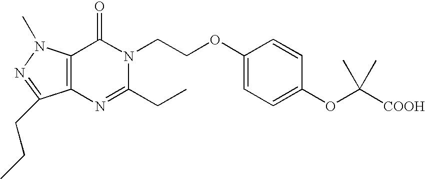

The title compound was prepared by following the same procedure as described in example 64 by treating 2-{4-[2-(5-ethyl-1-methyl-7-oxo-3-propyl-1,7-dihydro-pyrazolo[4,3-d]pyrimidin-6-yl)-ethoxy]-phenoxy}-2-methyl-propionic acid ethyl ester (195 mg, 0.41 mmol), obtained in example 69, in methanol (5 mL) using sodium carbonate (220 mg, 2.07 mmol) at 20 to 40° C. for 60 hours.

Yield: 160 mg, 88%.

Melting Point: 108-110° C.

¹H NMR (CDCl₃, 200 MHz): δ 6.88 (d, J=9.1 Hz, 2H), 6.75 (d, J=9.1 Hz, 2H), 4.49 (t, J=5.1 Hz, 2H), 4.47-4.24 (m, 5H), 3.04 (q, J=7.2 Hz, 2H), 2.85 (t, J=6.5 Hz, 2H), 1.90-1.71 (m, 2H), 1.52 (s, 6H), 1.37 (t, J=7.4 Hz, 3H), 0.98 (t, J=7.4 Hz, 3H).

Mass (CI): m/z 443 (M⁺+1).

IR (cm⁻¹) (KBr): 3432, 2929, 1702, 1688, 1570, 1504.

### EXAMPLE 71

### 2-{4-[3-(5-Ethyl-1-methyl-7-oxo-3-propyl-1,7-dihydro-pyrazolo[4,3-d]pyrimidin-6-yl)-propylamino]-phenoxy}-2-methyl-propionic acid ethyl ester

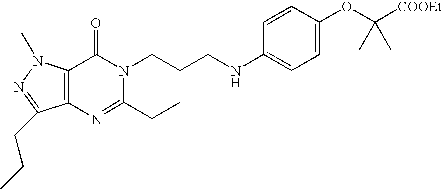

The title compound was prepared by following the same procedure as described in example 63, by heating 6-(3-bromopropyl)-1,5-dimethyl-3-propyl-6,7-dihydro-1H-pyrazolo[4,3-d]pyrimidin-7-one (400 mg, 1.17 mmol) and 2-(4-amino-phenoxy)-2-methyl-propionic acid ethyl ester (287 mg, 1.29 mmol), obtained in preparation 51, in the presence of potassium carbonate (535 mg, 3.87 mmol) and tetrabutylammonium bromide (20 mg, 0.062 mmol) in toluene (10 mL) at 90° C. for 48 hours.

Yield: 260 mg, 41%.

¹H NMR (CDCl₃, 200 MHz): δ 6.78 (d, J=8.6 Hz, 2H), 6.53 (d, J=8.6 Hz, 2H), 4.30-4.10 (m, 7H), 3.20 (t, J=5.9 Hz, 2H), 2.89-2.74 (m, 4H), 2.08-1.96 (m, 2H), 1.86-1.74 (m, 2H), 1.51 (s, 6H), 1.38-1.20 (m, 6H), 0.99 (t, J=7.2 Hz, 3H).

Mass (CI): m/z 484 (M⁺+1).

IR (cm⁻¹) (KBr): 2937, 2873, 1734, 1687, 1512.

### EXAMPLE 72

### 2-{4-[3-(5-Ethyl-1-methyl-7-oxo-3-propyl-1,7-dihydro-pyrazolo[4,3-d]pyrimidin-6-yl)-propylamino]-phenoxy}-2-methyl-propionic acid

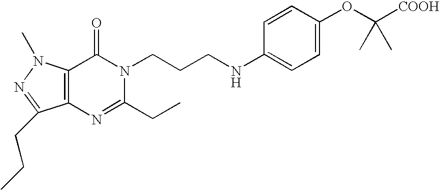

The title compound was prepared by following the same procedure as described in example 64 by treating 2-{4-[3-(5-ethyl-1-methyl-7-oxo-3-propyl-1,7-dihydro-pyrazolo[4,3-d]pyrimidin-6-yl)-propylamino]-phenoxy}-2-methyl-propionic acid ethyl ester (260 mg, 0.53 mmol), obtained in example 71, in methanol (5 mL) using sodium carbonate (285 mg, 2.68 mmol) at 20 to 40° C. for 72 hours.

Yield: 170 mg, 70%.

Melting Point: 124-126° C.

¹H NMR (CDCl₃, 200 MHz): δ 6.83 (d, J=8.8 Hz, 2H), 6.57 (d, J=8.6 Hz, 2H), 4.23 (s, 3H), 4.20 (t, J=7.2 Hz, 2H), 3.20 (t, J=6.1 Hz, 2H), 2.88-2.74 (m, 4H), 2.08-1.94 (m, 2H), 1.90-1.71 (m, 2H), 1.51 (s, 6H), 1.34 (t, J=7.2 Hz, 3H), 0.99 (t, J=7.3 Hz, 3H).

Mass (CI): m/z 456 (M⁺+1).

IR (cm⁻¹) (KBr): 3423, 2927, 1752, 1688, 1516.

### EXAMPLE 73

### 2-{4-[3-(5-Ethyl-1-methyl-7-oxo-3-propyl-1,7-dihydro-pyrazolo[4,3-d]pyrimidin-6-yl)-propoxy]-phenoxy}-2-methyl-propionic acid ethyl ester

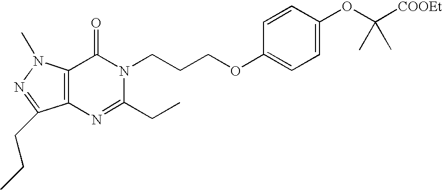

The title compound was prepared by following the same procedure as described in example 63, by heating 6-(3-bromopropyl)-1,5-dimethyl-3-propyl-6,7-dihydro-1H-pyrazolo[4,3-d]pyrimidin-7-one (400 mg, 1.17 mmol) and 2-(4-hydroxy-phenoxy)-2-methyl-propionic acid ethyl ester (290 mg, 1.29 mmol)), obtained in preparation 50, in the presence of potassium carbonate (535 mg, 3.87 mmol) and tetrabutylammonium bromide (180 mg, 0.59 mmol) in toluene (9 mL) at 90° C. for 48 hours.

Yield: 400 mg, 70%.

¹H NMR (CDCl₃, 200 MHz): δ 6.83 (d, J=9.1 Hz, 2H), 6.74 (d, J=9.1 Hz, 2H), 4.31-4.18 (m, 7H), 4.01 (t, J=5.5 Hz, 2H), 2.88-2.80 (m, 4H), 2.24-2.11 (m, 2H), 1.91-1.72 (m, 2H), 1.53 (s, 6H), 1.34 (t, J=6.4 Hz, 3H), 1.28 (t, J=7.0 Hz, 3H), 0.99 (t, J=7.4 Hz, 3H).

Mass (CI): m/z 485 (M⁺+1).

IR (cm⁻¹) (KBr): 2936, 1734, 1689, 1504.

### EXAMPLE 74

### 2-{4-[3-(5-Ethyl-1-methyl-7-oxo-3-propyl-1,7-dihydro-pyrazolo[4,3-d]pyrimidin-6-yl)-propoxy]-phenoxy}-2-methyl-propionic acid

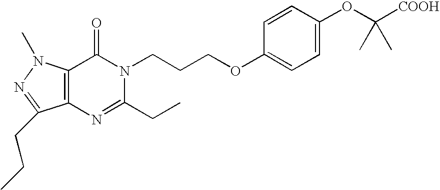

The title compound was prepared by following the same procedure as described in example 64 by treating 2-{4-[3-(5-ethyl-1-methyl-7-oxo-3-propyl-1,7-dihydro-pyrazolo[4,3-d]pyrimidin-6-yl)-propoxy]-phenoxy}-2-methyl-propionic acid ethyl ester (400 mg, 0.83 mmol), obtained in example 73, in methanol (7 mL) using sodium carbonate (440 mg, 4.13 mmol) at 20 to 40° C. for 72 hours.

Yield: 340 mg, 90%.

Melting Point: 124-126° C.

¹H NMR (CDCl₃, 200 MHz): δ 6.92 (d, J=9.1 Hz, 2H), 6.77 (d, J=9.1 Hz, 2H), 4.29 (t, J=7.4 Hz, 2H), 4.20 (s, 3H), 4.04 (t, J=5.4 Hz, 2H), 2.94-2.78 (m, 4H), 2.26-2.14 (m, 2H), 1.92-1.74 (m, 2H), 1.54 (s, 6H), 1.35 (t, J=7.4 Hz, 3H), 0.99 (t, J=7.4 Hz, 3H).

Mass (CI): m/z 457 (M⁺+1).

IR (cm⁻¹) (KBr): 3429, 2958, 1710, 1696, 1575, 1506.

### EXAMPLE 75

### 2-{4-[2-(5-Ethyl-1-methyl-7-oxo-3-propyl-1,7-dihydro-pyrazolo[4,3-d]pyrimidin-6-yl)-ethylamino]-phenoxy}-2-methyl-propionic acid ethyl ester

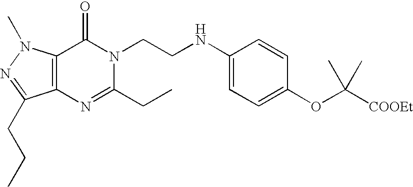

The title compound was prepared by following the same procedure as described in example 63, by heating 6-(2-chloroethyl)-5-ethyl-1-methyl-3-propyl-6,7-dihydro-1H-pyrazolo[4,3-d]pyrimidin-7-one (350 mg, 1.3 mmol) and 2-(4-amino-phenoxy)-2-methyl-propionic acid ethyl ester (318 mg, 1.43 mmol), obtained in preparation 51, in the presence of potassium carbonate (592 mg, 4.28 mmol) and tetrabutylammonium bromide (21 mg, 0.064 mmol) in toluene (8 mL) at 90° C. for 48 hours.

Yield: 380 mg, 52%.

¹H NMR (CDCl₃, 200 MHz): δ 6.78 (d, J=8.8 Hz, 2H), 6.53 (d, J=8.8 Hz, 2H), 4.33-4.17 (m, 7H), 3.46 (t, J=6.3 Hz, 2H), 2.87-2.72 (m, 4H), 1.84-1.76 (m, 2H), 1.50 (s, 6H), 1.35-1.24 (m, 6H), 0.98 (t, J=7.2 Hz, 3H).

Mass (CI): m/z 470 (M⁺+1).

IR (cm⁻¹) (KBr): 2937, 1734, 1688, 1513.

### EXAMPLE 76

### 2-{4-[2-(5-Ethyl-1-methyl-7-oxo-3-propyl-1,7-dihydro-pyrazolo[4,3-d]pyrimidin-6-yl)-ethylamino]-phenoxy}-2-methyl-propionic acid

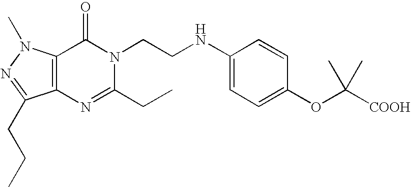

The title compound was prepared by following the same procedure as described in example 64 by treating 2-{4-[3-(5-ethyl-1-methyl-7-oxo-3-propyl-1,7-dihydro-pyrazolo[4,3-d]pyrimidin-6-yl)-propoxy]-phenoxy}-2-methyl-propionic acid (380 mg, 0.81 mmol), obtained in example 74, in methanol (6 mL) using sodium carbonate (430 mg, 4.05 mmol) at 20 to 40° C. for 48 hours.

Yield: 300 mg, 84%

Melting Point: 136-138° C.

¹H NMR (CDCl₃, 200 MHz): δ 6.82 (d, J=8.8 Hz, 2H), 6.57 (d, J=9.2 Hz, 2H), 4.32 (q, J=6.3 Hz, 2H), 4.24 (s, 3H), 3.48 (t, J=6.3 Hz, 2H), 2.89-2.76 (m, 4H), 1.92-1.72 (m, 2H), 1.49 (s, 6H), 1.33 (t, J=7.2 Hz, 3H), 1.00 (t, J=7.2 Hz, 3H).

Mass (CI): m/z 442 (M⁺+1).

IR (cm⁻¹) (KBr): 3529, 2931, 1710, 1697, 1572, 1515.

### EXAMPLE 77

### 2-{4-[3-(1,5-Dimethyl-7-oxo-3-propyl-1,7-dihydro-pyrazolo[4,3-d]pyrimidin-6-yl)-propyl]-phenoxy}-2-methyl-propionic acid ethyl ester

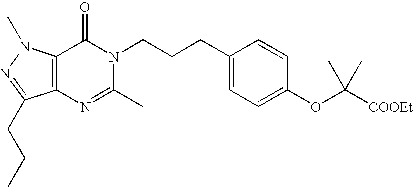

To a solution of 1,5-dimethyl-3-propyl-6,7-dihydro-1H-pyrazolo[4,3-d]pyrimidin-7-one (300 mg, 1.45 mmol) in dimethylformamide (4 mL) potassium carbonate (600 mg, 4.35 mmol) was added and stirred for 30 minutes. 2-[4-(3-methanesulfonyloxy-propyl)-phenoxy]-2-methyl-propionic acid ethyl ester (550 mg, 1.67 mmol) in dimethylformamide (2 mL) was added drop wise and heated the mixture at 50° C. for 24 hours. The reaction mixture was diluted with ethyl acetate and washed with water and brine, dried with sodium sulphate and evaporated to dryness. The crude mass was column purified using 20% ethyl acetate in pet ether to give pure title compound.

Yield: 336 mg, 51%.

¹H NMR (CDCl₃, 200 MHz): δ 7.09 (d, J=8.3 Hz, 2H), 6.80 (d, J=8.3 Hz, 2H), 4.24 (q, J=7.8 Hz, 2H), 4.23 (s, 3H), 4.02 (t, J=7.8 Hz, 2H), 2.82 (t, J=7.6 Hz, 2H), 2.70 (t, J=7.6 Hz, 2H), 2.44 (s, 3H), 2.04-1.95 (m, 2H), 1.84-1.73 (m, 2H), 1.57 (s, 6H), 1.26 (t, J=7.1 Hz, 3H), 0.98 (t, J=7.3 Hz, 3H).

Mass (CI): m/z 455 (M⁺+1).

IR (cm⁻¹) (KBr): 2961, 1734, 1690, 1509.

### EXAMPLE 781

### 2-{4-[3-(1,5-Dimethyl-7-oxo-3-propyl-1,7-dihydro-pyrazolo[4,3-d]pyrimidin-6-yl)-propyl]-phenoxy}-2-methyl-propionic acid

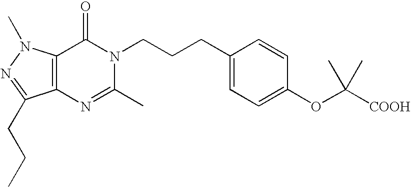

The title compound was prepared by treating 2-{4-[3-(1,5-dimethyl-7-oxo-3-propyl-1,7-dihydro-pyrazolo[4,3-d]pyrimidin-6-yl)-propyl]-phenoxy}-2-methyl-propionic acid ethyl ester (200 mg, 0.44 mmol), obtained in example 77, in ethanol (8 mL) using powdered potassium hydroxide (148 mg, 2.64 mmol) for 4.5 hours. The reaction mixture was acidified with acetic acid upto pH 5. Methanol was removed and scratched with chloroform. The solids were filtered off and the filtrate was evaporated to dryness. The residue was purified through a column using 10% methanol in chloroform to obtain title compound.

Yield: 110 mg, 59%.

¹H NMR (CDCl₃, 200 MHz): δ 7.13 (d, J=8.1 Hz, 2H), 6.89 (d, J=8.1 Hz, 2H), 4.22 (s, 3H), 4.05 (t, J=7.7 Hz, 2H), 2.85-2.68 (m, 6H), 2.46 (s, 3H), 2.04-1.83 (m, 2H), 1.79-1.72 (m, 2H), 1.58 (s, 6H), 0.98 (t, J=7.3 Hz, 3H).

Mass (CI): m/z 427 (M⁺+1).

IR (cm⁻¹) (KBr): 2934, 1710, 1692, 1574.

### EXAMPLE 79

### Argenine salt of 2-{4-[3-(1,5-dimethyl-7-oxo-3-propyl-1,7-dihydro-pyrazolo[4,3-d]pyrimidin-6-yl)-propyl]-phenoxy}-2-methyl-propionic acid

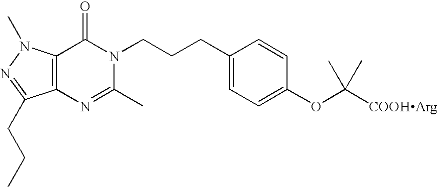

The title compound was prepared by treating 2-{4-[3-(1,5-dimethyl-7-oxo-3-propyl-1,7-dihydro-pyrazolo[4,3-d]pyrimidin-6-yl)-propyl]-phenoxy}-2-methyl-propionic acid (145 mg, 0.34 mmol), obtained in example 78, in methanol (3 mL) and argenine (59 mg, 0.34 mmol) at 20 to 40° C. for 20 hours.

Yield: 190 mg, 93%.

Melting Point: 104-106° C.

¹H NMR (CDCl₃, 200 MHz): δ 7.09 (d, J=8.9 Hz, 2H), 6.85 (d, J=8.4 Hz, 2H), 4.17 (s, 3H), 4.07 (t, J=8.0 Hz, 2H), 3.22-3.14 (m, 3H), 2.79 (t, J=7.5 Hz, 2H), 2.67 (t, J=7.6 Hz, 2H), 2.48 (s, 3H), 2.35 (t, J=6.5 Hz, 1H), 2.06-1.95 (m, 2H), 1.85-1.62 (m, 6H), 1.50 (s, 6H), 0.96 (t, J=7.4 Hz, 3H).

Mass (CI): m/z 601.5 (M⁺+1).

IR (cm⁻¹) (KBr): 2963, 1685, 1572, 1509.

### EXAMPLE 80

### 2-{3-[3-(1,5-Dimethyl-7-oxo-3-propyl-1,7-dihydro-pyrazolo[4,3-d]pyrimidin-6-yl)-propyl]-phenoxy}-2-methyl-propionic acid ethyl ester

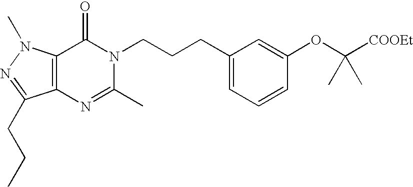

To a solution of 1,5-dimethyl-3-propyl-6,7-dihydro-1H-pyrazolo[4,3-d]pyrimidin-7-one (400 mg, 1.94 mmol) in dimethylformamide (5 mL) potassium carbonate (804 mg, 5.82 mmol) was added and stirred for 30 minutes. 2-[3-(3-methanesulfonyloxy-propyl)-phenoxy]-2-methyl-propionic acid ethyl ester (735 mg, 2.13 mmol) in dimethylformamide (2 mL) was added drop wise and heated the mixture at 50° C. for 24 hours. The reaction mixture was diluted with ethyl acetate and washed with water and brine, dried over sodium sulphate and evaporated to dryness. The crude mass was column purified using 22% ethyl acetate in pet ether to obtain title product

Yield: 460 mg, 52%.

¹H NMR (CDCl₃, 200 MHz): δ 7.16 (t, J=7.9 Hz, 1H), 6.85 (d, J=7.6 Hz, 1H), 6.67 (bs, 1H), 6.67 (d, J=8.4 Hz, 1H), 4.28-4.18 (m, 2H), 4.23 (s, 3H), 4.03 (t, J=8.0 Hz, 2H), 2.83 (t, J=7.7 Hz, 2H), 2.70 (t, J=7.4 Hz, 2H), 2.46 (s, 3H), 2.08-1.96 (m, 2H), 1.84-1.73 (m, 2H), 1.59 (s, 6H), 1.25 (t, J=7.0 Hz, 3H), 0.99 (t, J=7.4 Hz, 3H).

Mass (CI): m/z 455 (M⁺+1).

IR (cm⁻¹) (Neat): 2960, 1734, 1690, 1575, 1177.

### EXAMPLE 81

### 2-{3-[3-(1,5-Dimethyl-7-oxo-3-propyl-1,7-dihydro-pyrazolo[4,3-d]pyrimidin-6-yl)-propyl]-phenoxy}-2-methyl-propionic acid

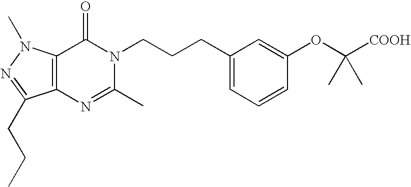

The title compound was prepared by following the same procedure as described in example 78 by treating 2-{3-[3-(1,5-dimethyl-7-oxo-3-propyl-1,7-dihydro-pyrazolo[4,3-d]pyrimidin-6-yl)-propyl]-phenoxy}-2-methyl-propionic acid ethyl ester (200 mg, 0.44 mmol), obtained in example 80, in ethanol (3 mL) using powdered potassium hydroxide (148 mg, 2.64 mmol) for 1.5 hours.

Yield: 130 mg, 70%.

¹H NMR (CDCl₃, 400 MHz): δ 7.10 (d, J=7.6 Hz, 2H), 6.80-6.72 (m, 3H), 4.15 (s, 3H), 3.93 (t, J=8.1 Hz, 2H), 2.80 (t, J=7.6 Hz, 2H), 2.66 (t, J=6.9 Hz, 2H), 2.48 (s, 3H), 1.97-1.92 (m, 2H), 1.82-1.70 (m, 2H), 1.53 (s, 6H), 0.97 (t, J=7.4 Hz, 3H).

Mass (CI): m/z 427 (M⁺+1).

IR (cm⁻¹) (Neat): 2934, 1710, 1690, 1575.

### EXAMPLE 82

### Argenine salt of 2-{3-[3-(1,5-dimethyl-7-oxo-3-propyl-1,7-dihydro-pyrazolo[4,3-d]pyrimidin-6-yl)-propyl]-phenoxy}-2-methyl-propionic acid

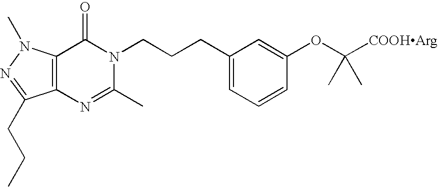

The title compound was prepared by treating 2-{3-[3-(1,5-dimethyl-7-oxo-3-propyl-1,7-dihydro-pyrazolo[4,3-d]pyrimidin-6-yl)-propyl]-phenoxy}-2-methyl-propionic acid (95 mg, 0.223 mmol), obtained in example 81, in methanol (2 mL) and argenine (38 mg, 0.223 mmol) at 20 to 40° C. for 20 hours.

Yield: 119 mg, 89%.

Melting Point: 78-80° C.

¹H NMR (CD₃OD, 400 MHz): δ 7.11 (d, J=7.8 Hz, 1H), 6.82-6.74 (m, 3H), 4.17 (s, 3H), 4.10-4.07 (m, 2H), 3.49 (t, J=6.0 Hz, 1H), 3.29-3.14 (m, 2H), 2.78 (t, J=7.5 Hz, 2H), 2.69 (t, J=7.4 Hz, 2H), 2.48 (s, 3H), 2.02-1.82 (m, 2H), 1.79-1.71 (m, 2H), 1.71-1.66 (m, 4H), 1.50 (s, 6H), 0.96 (t, J=7.4 Hz, 3H).

Mass (ES): m/z 601.7 (M⁺+1).

IR (cm⁻¹) (Neat): 2925, 1690, 1573.

### EXAMPLE 83

### 2-{4-[3-(5-Ethyl-1-methyl-7-oxo-3-propyl-1,7-dihydro-pyrazolo[4,3-d]pyrimidin-6-yl)-propyl]-phenoxy}-2-methyl-propionic acid ethyl ester

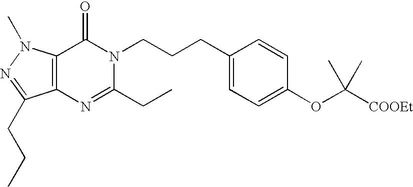

To a solution of 5-ethyl-1-methyl-3-propyl-6,7-dihydro-1H-pyrazolo[4,3-d]pyrimidin-7-one (400 mg, 1.82 mmol) in dimethylformamide (4 mL) potassium carbonate (955 mg, 6.91 mmol) was added and stirred for 30 minutes. 2-[4-(3-methanesulfonyloxy-propyl)-phenoxy]-2-methyl-propionic acid ethyl ester (876 mg, 2.54 mmol) in dimethylformamide (2 mL) was added drop wise and heated the mixture at 50° C. for 24 hours. The reaction mixture was diluted with ethyl acetate and washed with water and brine, dried over sodium sulphate and evaporated to dryness. The crude mass was column purified using 20% ethyl acetate in pet ether to obtain pure title compound.

Yield: 319 mg, 38%.

¹H NMR (CDCl₃, 200 MHz): δ 7.16 (t, J=7.9 Hz, 1H), 6.85 (d, J=7.6 Hz, 1H), 6.67 (bs, 1H), 6.67 (d, J=8.4 Hz, 1H), 4.28-4.18 (m, 2H), 4.23 (s, 3H), 4.03 (t, J=8.0 Hz, 2H), 2.83 (t, J=7.7 Hz, 2H), 2.70 (t, J=7.4 Hz, 2H), 2.46 (s, 3H), 2.08-1.96 (m, 2H), 1.84-1.73 (m, 2H), 1.59 (s, 6H), 1.25 (t, J=7.0 Hz, 3H), 0.99 (t, J=7.4 Hz, 3H).

Mass (CI): m/z 455 (M⁺+1).

IR (cm⁻¹) (KBr): 2960, 1734, 1690, 1575, 1177.

### EXAMPLE 84

### 2-{4-[3-(5-Ethyl-1-methyl-7-oxo-3-propyl-1,7-dihydro-pyrazolo[4,3-d]pyrimidin-6-yl)-propyl]-phenoxy}-2-methyl-propionic acid

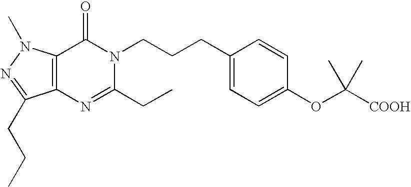

The title compound was prepared by following the same procedure as described in example 78 by treating 2-{4-[3-(5-ethyl-1-methyl-7-oxo-3-propyl-1,7-dihydro-pyrazolo[4,3-d]pyrimidin-6-yl)-propyl]-phenoxy}-2-methyl-propionic acid ethyl ester (315 mg, 0.67 mmol), obtained in example 83, in ethanol (4 mL) using powdered potassium hydroxide (226 mg, 4.04 mmol) for 1 hours.

Yield: 107 mg, 36%.

¹H NMR (CDCl₃, 400 MHz): δ 7.13 (d, J=8.1 Hz, 2H), 6.89 (d, J=8.1 Hz, 2H), 4.22 (s, 3H), 4.05 (t, J=7.7 Hz, 2H), 2.85-2.68 (m, 6H), 2.46 (s, 3H), 2.04-1.83 (m, 2H), 1.79-1.72 (m, 2H), 1.58 (s, 6H), 0.98 (t, J=7.3 Hz, 3H).

Mass (CI): m/z 427 (M⁺+1).

IR (cm⁻¹) (KBr): 2934, 1710, 1692, 1574.

### EXAMPLE 85

Argenine salt of 2-{4-[3-(5-ethyl-1-methyl-7-oxo-3-propyl-1,7-dihydro-pyrazolo[4,3-d]pyrimidin-6-yl)-propyl]-phenoxy}-2-methyl-propionic acid

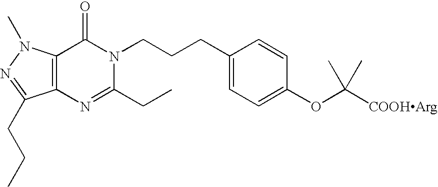

The title compound was prepared by treating 2-{4-[3-(5-ethyl-1-methyl-7-oxo-3-propyl-1,7-dihydro-pyrazolo[4,3-d]pyrimidin-6-yl)-propyl]-phenoxy}-2-methyl-propionic acid (36 mg, 0.08 mmol), obtained in example 84, in methanol (2 mL) argenine (14 mg, 0.08 mmol) was added and the reaction mixture was stirred at 20 to 40° C. for 20 hours.

Yield: 48 mg, 96%.

Melting Point: 100-102° C.

¹H NMR (CD₃OD, 400 MHz): δ 7.08 (d, J=8.6 Hz, 1H), 6.86 (d, J=8.6 Hz, 2H), 4.17 (s, 3H), 4.09-4.04 (m, 2H), 3.51 (t, J=6.2 Hz, 1H), 3.26-3.13 (m, 2H), 2.80 (t, J=7.4 Hz, 2H), 2.71-2.65 (m, 4H), 2.00-1.95 (m, 2H), 1.82 (t, J=6.9 Hz, 2H), 1.77 (t, J=7.5 Hz, 2H), 1.73-1.68 (m, 4H), 1.49 (s, 6H), 1.26-1.22 (m, 3H), 0.96 (t, J=7.4 Hz, 3H).

IR (cm⁻¹) (KBr): 2931, 1685, 1555.

### EXAMPLE 86

### 2-{3-[3-(1,5-Dimethyl-7-oxo-3-propyl-1,7-dihydro-pyrazolo[4,3-d]pyrimidin-6-yl)-propyl]-phenoxy}-2-methyl-butyric acid ethyl ester

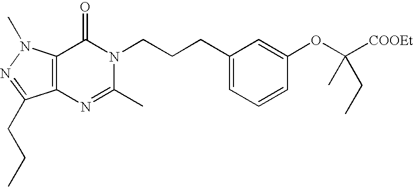

To a solution of 1,5-dimethyl-3-propyl-6,7-dihydro-1H-pyrazolo[4,3-d]pyrimidin-7-one (366 mg, 1.78 mmol) in dimethylformamide (6 mL) potassium carbonate (737 mg, 5.33 mmol) was added and stirred for 30 minutes. 2-[3-(3-methanesulfonyloxy-propyl)-phenoxy]-2-methyl-butyric acid ethyl ester (700 mg, 1.96 mmol) in dimethylformamide (2 mL) was added drop wise and heated the mixture at 50° C. for 24 hours. The reaction mixture was diluted with ethyl acetate and washed with water and brine, dried over sodiumsulphate and evaporated to dryness. The crude mass was column purified using 20% ethyl acetate in pet ether to give title product.

Yield: 330 mg, 40%.

¹H NMR (CDCl₃, 200 MHz): δ 7.15 (t, J=7.8 Hz, 1H), 6.84 (d, J=7.6 Hz, 1H), 6.74-6.65 (m, 2H), 4.24 (q, J=7.0 Hz, 2H), 4.23 (s, 3H), 4.03 (t, J=7.8 Hz, 2H), 2.82 (t, J=7.7 Hz, 2H), 2.70 (t, J=7.4 Hz, 2H), 2.45 (s, 3H), 2.07-1.80 (m, 4H), 1.76-1.69 (m, 2H), 1.49 (s, 3H), 1.25 (t, J=7.2 Hz, 3H), 0.98 (t, J=7.3 Hz, 6H).

Mass (CI): m/z 469 (M⁺+1).

IR (cm⁻¹) (Neat): 2964, 1733, 1690, 1575.

### EXAMPLE 87

### 2-{3-[3-(1,5-Dimethyl-7-oxo-3-propyl-1,7-dihydro-pyrazolo[4,3-d]pyrimidin-6-yl)-propyl]-phenoxy}-2-methyl-butyric acid

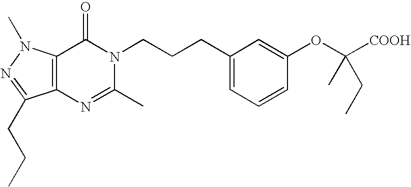

The title compound was prepared by following the same procedure as described in example 78 by treating 2-{3-[3-(1,5-dimethyl-7-oxo-3-propyl-1,7-dihydro-pyrazolo[4,3-d]pyrimidin-6-yl)-propyl]-phenoxy}-2-methyl-butyric acid ethyl ester (330 mg, 0.70 mmol), obtained in example 86, in ethanol (6 mL) using powdered potassium hydroxide (237 mg, 4.23 mmol) for 4 hours.

Yield: 90 mg, 29%.

Melting Point: 136-138° C.

¹H NMR (CDCl₃, 400 MHz): δ 7.21 (d, J=7.8 Hz, 1H), 6.92 (d, J=7.8 Hz, 1H), 6.85-6.80 (m, 2H), 4.22 (s, 3H), 4.03-3.98 (m, 2H), 2.82 (t, J=7.6 Hz, 2H), 2.74 (t, J=7.2 Hz, 2H), 2.49 (s, 3H), 2.07-1.94 (m, 4H), 1.93-1.75 (m, 2H), 1.51 (s, 3H), 1.05 (t, J=7.4 Hz, 3H), 0.98 (t, J=7.4 Hz, 3H).

Mass (CI): m/z 441 (M⁺+1).

IR (cm⁻¹) (Neat): 3421, 2873, 1710, 1692, 1577.

### EXAMPLE 88

### 2-{3-[2-(1,5-Dimethyl-7-oxo-3-propyl-1,7-dihydro-pyrazolo[4,3-d]pyrimidin-6-yl)-ethoxy]-phenoxy}-2-methyl-propionic acid ethyl ester

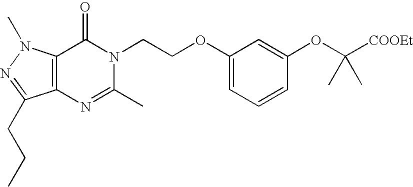

The title compound was prepared by following the same procedure as described in example 63, by heating 6-(2-chloroethyl)-1,5-dimethyl-3-propyl-6,7-dihydro-1H-pyrazolo[4,3-d]pyrimidin-7-one (500 mg, 1.87 mmol) and 2-(3-hydroxy-phenoxy)-2-methyl-propionic acid ethyl ester (348 mg, 1.55 mmol), obtained in preparation 52, in the presence of potassium carbonate (705 mg, 5.11 mmol) and tetrabutylammonium bromide (25 mg, 0.078 mmol) in toluene (9 mL) at 90° C. for 48 hours.

Yield: 585 mg, 69%.

Melting Point: 86-88° C.

¹H NMR (CDCl₃, 200 MHz): δ 7.08 (t, J=8.4 Hz, 1H), 6.51-6.41 (m, 1H), 6.40-6.37 (m, 2H), 4.47 (t, J=5.1 Hz, 2H), 4.26 (t, J=5.1 Hz, 2H), 4.24-4.18 (m, 2H), 4.22 (s, 3H), 2.84 (t, J=7.6 Hz, 2H), 2.74 (s, 3H), 1.85-1.75 (m, 2H), 1.57 (s, 6H), 1.23 (t, J=7.1 Hz, 3H), 1.00 (t, J=7.4 Hz, 3H).

Mass (CI): m/z 457 (M⁺+1).

IR (cm⁻¹) (KBr): 2961, 1734, 1690, 1576, 1486.

### EXAMPLE 89

### 2-{3-[2-(1,5-Dimethyl-7-oxo-3-propyl-1,7-dihydro-pyrazolo[4,3-d]pyrimidin-6-yl)-ethoxy]-phenoxy}-2-methyl-propionic acid

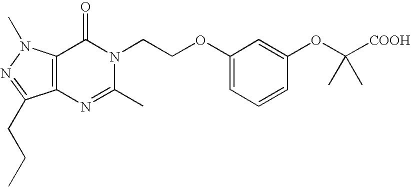

The title compound was prepared by following the same procedure as described in example 64 by treating 2-{3-[2-(1,5-dimethyl-7-oxo-3-propyl-1,7-dihydro-pyrazolo[4,3-d]pyrimidin-6-yl)-ethoxy]-phenoxy}-2-methyl-propionic acid ethyl ester (300 mg, 0.66 mmol), obtained in example 88, in methanol:water (10 mL, 1:1 ratio) using sodium carbonate (348 mg, 3.28 mmol) for 72 hours.

Yield: 190 mg, 68%.

Melting Point: 140-142° C.

¹H NMR (CDCl₃, 200 MHz): δ 7.12 (t, J=8.2 Hz, 1H), 6.57-6.50 (m, 2H), 6.48 (d, J=2.1 Hz, 1H), 4.46 (t, J=5.1 Hz, 2H), 4.27 (t, J=5.1 Hz, 2H), 4.21 (s, 3H), 2.82 (t, J=7.6 Hz, 2H), 2.73 (s, 3H), 1.81-1.73 (m, 2H), 1.55 (s, 6H), 0.98 (t, J=7.4 Hz, 3H).

Mass (CI): m/z 429 (M⁺+1).

IR (cm⁻¹) (KBr): 3430, 2932, 1729, 1689, 1579.

### EXAMPLE 90

### 1-{4-[2-(1,5-Dimethyl-7-oxo-3-propyl-1,7-dihydro-pyrazolo[4,3-d]pyrimidin-6-yl)-ethoxy]-phenoxy}-cyclobutanecarboxylic acid ethyl ester

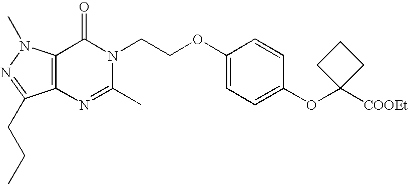

The title compound was prepared by following the same procedure as described in example 63, by heating 6-(2-chloroethyl)-1,5-dimethyl-3-propyl-6,7-dihydro-1H-pyrazolo[4,3-d]pyrimidin-7-one (500 mg, 1.86 mmol) and ethyl 1-(4-hydroxyphenoxy)-1-cyclobutanecarboxylate (462 mg, 1.95 mmol), obtained in preparation 53, in the presence of potassium carbonate (772 mg, 5.59 mmol) and tetrabutylammonium bromide (30 mg, 0.09 mmol) in toluene (9 mL) at 90° C. for 48 hours.

Yield: 459 mg, 53%.

Melting Point: 132-134° C.

¹H NMR (CDCl₃, 200 MHz): δ 6.72 (dd, J=6.8 and 2.3 Hz, 2H), 6.59 (dd, J=6.8 and 2.3 Hz, 2H), 4.46 (t, J=5.1 Hz, 2H), 4.23 (t, J=5.2 Hz, 2H), 4.21 (s, 3H), 4.17 (q, J=7.1 Hz, 2H), 2.83 (t, J=7.6 Hz, 2H), 2.74 (s, 3H), 2.72-2.65 (m, 2H), 2.43-2.35 (m, 2H), 2.04-1.92 (m, 2H), 1.83-1.75 (m, 2H), 1.16 (t, J=7.0 Hz, 3H), 1.00 (t, J=7.4 Hz, 3H).

Mass (CI): m/z 469 (M⁺+1).

IR (cm⁻¹) (KBr): 2958, 1728, 1696, 1573, 1506.

### EXAMPLE 91

### 1-{4-[2-(1,5-Dimethyl-7-oxo-3-propyl-1,7-dihydro-pyrazolo[4,3-d]pyrimidin-6-yl)-ethoxy]-phenoxy}-cyclobutanecarboxylic acid

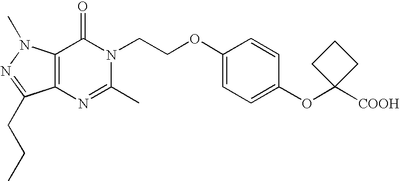

The title compound was prepared by following the same procedure as described in example 64 by treating 1-{4-[2-(1,5-dimethyl-7-oxo-3-propyl-1,7-dihydro-pyrazolo[4,3-d]pyrimidin-6-yl)-ethoxy]-phenoxy}-cyclobutanecarboxylic acid ethyl ester (275 mg, 0.58 mmol), obtained in example 90, in methanol:water (8 mL, 1:1 ratio) using sodium carbonate (311 mg, 2.93 mmol) for 11 days.

Yield: 163 mg, 64%.

Melting Point: 188-190° C.

¹H NMR (CDCl₃, 400 MHz): δ 6.74 (dd, J=6.8 and 2.2 Hz, 2H), 6.64 (dd, J=6.9 and 2.3 Hz, 2H), 4.46 (t, J=5.1 Hz, 2H), 4.23 (t, J=5.1 Hz, 2H), 4.20 (s, 3H), 2.82 (t, J=7.6 Hz, 2H), 2.75-2.68 (m, 2H), 2.73 (s, 3H), 2.56-2.38 (m, 2H), 2.07-1.92 (m, 2H), 1.83-1.73 (m, 2H), 0.98 (t, J=7.4 Hz, 3H).

Mass (CI): m/z 441 (M⁺+1).

IR (cm⁻¹) (KBr): 3429, 2926, 1705, 1691, 1577, 1507.

### EXAMPLE 92

### 2-{3-[2-(1-Ethyl-5-methyl-7-oxo-3-propyl-1,7-dihydro-pyrazolo[4,3-d]pyrimidin-6-yl)-ethoxy]-phenoxy}-2-methyl-propionic acid ethyl ester

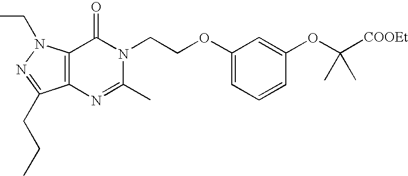

The title compound was prepared by following the same procedure as described in example 63, by heating 6-(2-bromoethyl)-1-ethyl-5-methyl-3-propyl-6,7-dihydro-1H-pyrazolo[4,3-d]pyrimidin-7-one (484 mg, 1.71 mmol) and 2-(3-hydroxy-phenoxy)-2-methyl-propionic acid ethyl ester (320 mg, 1.43 mmol), obtained in preparation 52, in the presence of potassium carbonate (591 mg, 4.29 mmol) and tetrabutylammonium bromide (46 mg, 0.14 mmol) in toluene (8 mL) at 90° C. for 48 hours.

Yield: 145 mg, 22%.

¹H NMR (CDCl₃, 400 MHz): δ 7.09 (t, J=8.5 Hz, 1H), 6.51-6.48 (m, 2H), 6.41-6.37 (m, 1H), 4.60 (q, J=7.2 Hz, 2H), 4.48 (t, J=5.1 Hz, 2H), 4.27 (t, J=5.1 Hz, 2H), 4.22 (q, J=7.2 KHz, 2H), 2.85 (t, J=7.7 Hz, 2H), 2.74 (s, 3H), 1.85-1.78 (m, 2H), 1.57 (s, 6H), 1.47 (t, J=7.2 Hz, 3H), 1.23 (t, J=7.1 Hz, 3H), 0.96 (t, J=7.4 Hz, 3H).

Mass (CI): m/z 471 (M⁺+1).

IR (cm⁻¹) (KBr): 2963, 1735, 1689, 1575.

### EXAMPLE 93

### 2-{3-[2-(1-Ethyl-5-methyl-7-oxo-3-propyl-1,7-dihydro-pyrazolo[4,3-d]pyrimidin-6-yl)-ethoxy]-phenoxy}-2-methyl-propionic acid

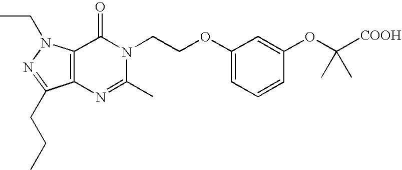

The title compound was prepared by following the same procedure as described in example 64 by treating 2-{3-[2-(1-ethyl-5-methyl-7-oxo-3-propyl-1,7-dihydro-pyrazolo[4,3-d]pyrimidin-6-yl)-ethoxy]-phenoxy}-2-methyl-propionic acid ethyl ester (175 mg, 0.37 mmol), obtained in example 92, in methanol:water (6 mL, 5:1 ratio) using sodium carbonate (197 mg, 1.86 mmol) for 6 days.

Yield: 65 mg, 39%).

Melting Point: 147-149° C.

¹H NMR (CDCl₃, 200 MHz): δ 7.12 (t, J=8.2 Hz, 1H), 6.58-6.47 (m, 3H), 4.59 (q, J=7.3 Hz, 2H), 4.47 (t, J=5.2 Hz, 2H), 4.28 (t, J=5.1 Hz, 2H), 2.83 (t, J=7.7 Hz, 2H), 2.74 (s, 3H), 1.82-1.75 (m, 2H), 1.58 (s, 6H), 1.46 (t, J=7.1 Hz, 3H), 0.98 (t, J=7.4 Hz, 3H).

Mass (CI): m/z 443 (M⁺+1).

IR (cm⁻¹) (KBr): 3430, 2935, 1710, 1689, 1550.

### EXAMPLE 94

### 2-{4-[3-(1,5-Dimethyl-7-oxo-3-propyl-1,7-dihydro-pyrazolo[4,3-d]pyrimidin-6-yl)-propyl]-phenoxy}-2-methyl-butyric acid ethyl ester

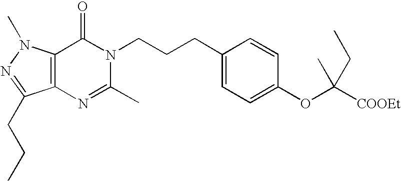

To a solution of 1,5-dimethyl-3-propyl-6,7-dihydro-1H-pyrazolo[4,3-d]pyrimidin-7-one (139 mg, 0.67 mmol) in dimethylformamide (4 mL) potassium carbonate (280 mg, 2.02 mmol) was added and stirred for 30 minutes. 2-[4-(3-methanesulfonyloxy-propyl)-phenoxy]-2-methyl-butyric acid ethyl ester (265 mg, 0.74 mmol) in dimethylformamide (1 mL) was added drop wise and heated the mixture at 50° C. for 24 hours. The reaction mixture was diluted with ethyl acetate and washed with water and brine, dried over sodiumsulphate and evaporated to dryness. The crude mass was column purified using 20% ethyl acetate in pet ether to give title compound

Yield: 90 mg, 29%.

¹H NMR (CDCl₃, 400 MHz): δ 7.07 (d, J=8.6 Hz, 2H), 6.80 (d, J=8.6 Hz, 2H), 4.23 (q, J=6.0 Hz, 2H), 4.22 (s, 3H), 4.02 (t, J=8.1 Hz, 2H), 2.82 (t, J=7.6 Hz, 2H), 2.69 (t, J=7.5 Hz, 2H), 2.43 (s, 3H), 2.05-1.94 (m, 4H), 1.81-1.76 (m, 2H), 1.46 (s, 3H), 1.29-1.24 (m, 6H), 1.00-0.96 (m, 3H).

Mass (CI): m/z 469 (M⁺+1).

IR (cm⁻¹) (KBr): 2931, 1734, 1690, 1573.

### EXAMPLE 95

### 2-{4-[3-(1,5-Dimethyl-7-oxo-3-propyl-1,7-dihydro-pyrazolo[4,3-d]pyrimidin-6-yl)-propyl]-phenoxy}-2-methyl-butyric acid

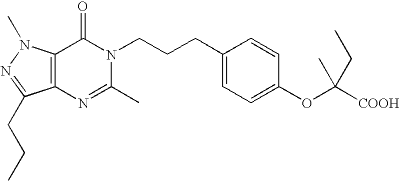

The title compound was prepared by following the same procedure as described in example 2 by hydrolyzing 2-{4-[3-(1,5-dimethyl-7-oxo-3-propyl-1,7-dihydro-pyrazolo[4,3-d]pyrimidin-6-yl)-propyl]-phenoxy}-2-methyl-butyric acid ethyl ester (90 mg, 0.19 mmol), obtained in example 94, in methanol (3 mL) using lithium hydroxide monohydrate (32 mg, 57.16 mmol) in water (1 mL) at 20 to 40° C. for 36 hours.

Yield: 57 mg, 67%.

¹H NMR (CDCl₃, 400 MHz): δ 7.14 (d, J=8.3 Hz, 2H), 6.90 (d, J=8.5 Hz, 2H), 4.22 (s, 3H), 4.05 (t, J=7.9 Hz, 2H), 2.82 (t, J=7.6 Hz, 2H), 2.71 (t, J=7.8 Hz, 2H), 2.47 (s, 3H), 2.03-1.99 (m, 2H), 1.95-1.75 (m, 4H), 1.45 (s, 3H), 1.04 (t, J=7.4 Hz, 3H), 0.98 (t, J=7.2 Hz, 3H).

Mass (CI): m/z 441 (M⁺+1).

IR (cm⁻¹) (Neat): 2931, 1710, 1692, 1575.

### EXAMPLE 96

### Magnesium salt of 2-{4-[3-(1,5-dimethyl-7-oxo-3-propyl-1,7-dihydro-pyrazolo[4,3-d]pyrimidin-6-yl)-propyl]-phenoxy}-2-methyl-butyric acid

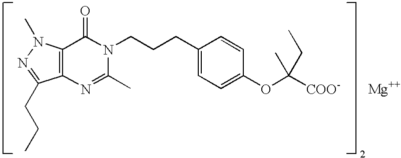

The title compound was prepared by treating 2-{4-[3-(1,5-dimethyl-7-oxo-3-propyl-1,7-dihydro-pyrazolo[4,3-d]pyrimidin-6-yl)-propyl]-phenoxy}-2-methyl-butyric acid (55 mg, 0.125 mmol), obtained in example 95, in dry methanol with magnesium hydroxide (4 mg, 0.061 mmol) for 18 hours at reflux temperature.

Yield: 52 mg, 92%

¹H NMR (CDCl₃, 400 MHz): δ 7.09 (d, J=8.5 Hz, 2H), 6.87 (d, J=8.5 Hz, 2H), 4.16 (s, 3H), 4.07 (t, J=7.9 Hz, 2H), 2.78 (t, J=7.5 Hz, 2H), 2.67 (t, J=7.4 Hz, 2H), 2.47 (s, 3H), 2.06-1.83 (m, 4H), 1.76-1.68 (m, 2H), 1.39 (s, 3H), 0.99-0.94 (m, 36).

Mass (CI): m/z 903.3 (M⁺+1).

IR (cm⁻¹) (Neat): 2934, 1690, 1615, 1573.

### EXAMPLE 97

### 2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid ethyl ester

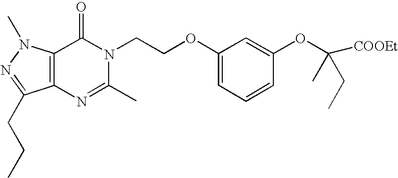

The title compound was prepared by following the same procedure as described in example 63, by heating 6-(2-chloroethyl)-1,5-dimethyl-3-propyl-6,7-dihydro-1H-pyrazolo[4,3-d]pyrimidin-7-one (500 mg, 1.87 mmol) and 2-(3-Hydroxy-phenoxy)-2-methyl-butyric acid ethyl ester (488 mg, 2.05 mmol), obtained in preparation 54, in the presence of potassium carbonate (772 mg, 5.59 mmol) and tetrabutylammonium bromide (30 mg, 0.093 mmol) in toluene (10 mL) at 90-100° C. for 48 hours.

Yield: 605 mg, 69%

¹H NMR (CDCl₃, 200 MHz): δ 7.08 (t, J=8.4 Hz, 1H), 6.50-6.41 (m, 3H), 4.47 (t, J=4.8 Hz, 2H), 4.28-4.17 (m, 4H), 4.22 (s, 3H), 2.84 (t, J=7.8 Hz, 2H), 2.75 (s, 3H), 2.02-1.81 (m, 2H), 1.78-1.68 (m, 2H), 1.48 (s, 3H), 1.23 (t, J=7.0 Hz, 3H), 1.00 (t, J=7.3 Hz, 3H), 0.96 (t, J=7.5 Hz, 3H).

Mass (CI): m/z 471 (M⁺+1).

IR (cm⁻¹) (KBr): 2964, 1733, 1690, 1577, 1487.

### EXAMPLE 98

### 2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid

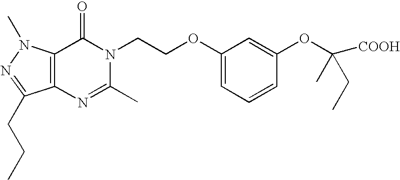

The title compound was prepared by following the same procedure as described in example 64 by treating 2-{3-[2-(1,5-dimethyl-7-oxo-3-propyl-1,7-dihydro-pyrazolo[4,3-d]pyrimidin-6-yl)-ethoxy]-phenoxy}-2-methyl-butyric acid ethyl ester (605 mg, 1.28 mmol), obtained in example 97, in methanol (6 mL) using sodium carbonate (682 mg, 6.43 mmol) at 20 to 40° C. for 15 days.

Yield: 233 mg, 41%.

Melting Point: 130-132° C.

¹H NMR (CDCl₃, 400 MHz): δ 7.13 (t, J=8.2 Hz, 1H), 6.58-6.48 (m, 3H), 4.47 (t, J=5.2 Hz, 2H), 4.23 (t, J=5.2 Hz, 2H), 4.21 (s, 3H), 2.82 (t, J=7.6 Hz, 2H), 2.74 (s, 3H), 2.05-1.90 (m, 2H), 1.83-1.74 (m, 2H), 1.48 (s, 3H), 1.02 (t, J=7.5 Hz, 3H), 0.98 (t, J=7.4 Hz, 3H).

Mass (CI): m/z 443 (M⁺+1).

IR (cm⁻¹) (KBr): 3433, 2928, 1710, 1698, 1601, 1488.

### EXAMPLE 99

### R-(+)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid methyl ester

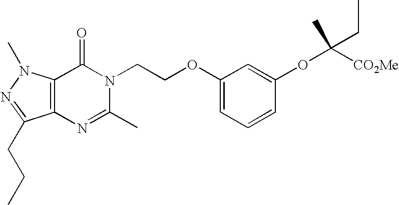

R-(+)-2-(3-hydroxyphenoxy)-2-methyl butyric acid methyl ester (140 mg, 0.62 mmol), obtained in preparation 59, dissolved in toluene (8 ml), was added with anhydrous potassium carbonate (0.25 grams, 1.81 mmol), and refluxed for about 1.0 hour using Dean-Stark water separator to remove water content of the reaction mixture. After cooling to 20 to 40° C., 6-(2-chloroethyl)-1,5-dimethyl-3-propyl-6,7-dihydro-1H-pyrazolo[4,3-d]pyrimidin-7-one (150 mg, 0.54 mmol) and tetra-n-butylammonium bromide (34 mg, 0.1 mmol) were subsequently added, and the reaction mixture was further refluxed for 20 hours. The reaction mixture was cooled to 20 to 40° C., poured over ice-water, stirred and extracted with ethyl acetate (3×10 mL). The combined organic layer was washed with water, dried over anhydrous sodium sulphate and evaporated to get a brown colored gummy mass which was purified by column chromatography using 230-400 mesh silica-gel and mixture of ethyl acetate and petroleum ether (50:50) to afford light brown syrup of the title compound.

Yield: 250 mg, 88%.

¹H NMR (CDCl₃, 400 MHz): δ 7.08 (t, J=7.6 Hz, 1H), 6.49 (dd, J=5.6 & 1.2 Hz, 1H), 6.93 (dd, J=2.4 & 1.6 Hz, 1H), 6.37 (dd, J=7.2 & 1.6 Hz, 1H), 4.46 (t, J=5.2 Hz, 2H), 4.26 (t, J=5.2 Hz, 2H), 4.21 (s, 3H), 3.74 (s, 3H), 2.83 (t, J=7.2 Hz, 2H), 2.74 (s, 3H), 2.04-1.90 (m, 2H), 1.85-1.75 (m, 2H), 1.48 (s, 3H), 1.05-0.96 (m, 6H).

Mass (CI): m/z 457 (M⁺+1).

IR (cm⁻¹) (KBr): 2960, 1736, 1690, 1575, 1487.

### EXAMPLE 100

### R-(+)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid

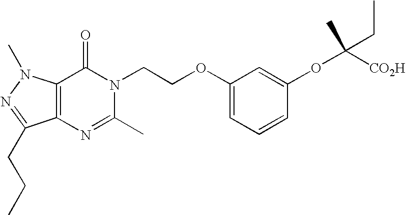

R-(+)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid methyl ester (245 mg, 0.53 mmol), obtained in example 99, dissolved in a mixture of acetone and water (1:1, 10 mL), was added with sodium hydroxide (42 mg, 1.00 mmol) at 20 to 40° C. The reaction mixture was refluxed for 1.5 hours, and then cooled to 20 to 40° C., poured over ice-water, stirred, acidified with 6 N hydrochloric acid, and extracted with ethyl acetate (3×10 mL). The combined organic layer was washed with water, dried over anhydrous sodium sulphate and evaporated to get a off white gummy mass which was purified by column chromatography using 230-400 mesh silica-gel and mixture of ethyl acetate and petroleum ether (60:40). A further trituration of the gummy mass with ethyl acetate and petroleum ether (50:50) yielded a colorless solid of the title compound.

Yield: 200 mg, 84%.

Melting Point: 118-120° C.

¹H NMR (CDCl₃, 200 MHz): δ 7.12 (t, J=8.4 Hz, 1H), 6.60-6.50 (m, 2H), 6.49-6.48 (m, 1H), 4.46 (t, J=5.2 Hz, 2H), 4.27 (t, J=5.2 Hz, 2H), 4.21 (s, 3H), 2.85 (t, J=7.6 Hz, 2H), 2.73 (s, 3H), 2.10-1.90 (m, 2H), 1.80-1.77 (m, 2H), 1.48 (s, 3H), 1.03-0.95 (m, 6H).

Mass (CI): m/z 443 (M⁺+1).

IR (cm⁻¹) (KBr): 3440, 2964, 1688, 1601, 1492, 1469.

[α]²⁵_{D}+5.6 (c=1.0%, MeOH).

### EXAMPLE 101

### R-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid methyl ester

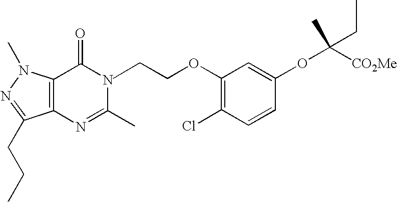

R-2-(4-Chloro-3-hydroxy-phenoxy)-2-methyl butyric acid methyl ester (1.20 grams, 4.65 mmol), obtained in preparation 65, dissolved in toluene (15 ml), was added with anhydrous potassium carbonate (1.24 grams, 8.98 mmol), and refluxed for about 1.0 hour using Dean-Stark water separator to remove water content of the reaction mixture. After cooling to 20 to 40° C., 6-(2-chloroethyl)-1,5-dimethyl-3-propyl-1,6-dihydropyrazolo[4,3-d]pyrimidin-7-one (1.18 g, 4.41 mmol) and tetra-n-butylammonium bromide (240 mg, 0.72 mmol) were subsequently added, and the reaction mixture was further refluxed for 20 hours. The reaction mixture was cooled to 20 to 40° C., poured over ice-water, stirred and extracted with ethyl acetate (3×20 mL). The combined organic layer was washed with water, dried (anhydrous Na₂SO₄) and evaporated to get a brown colored gummy mass which was purified by column chromatography using 230-400 mesh silica-gel and mixture of ethyl acetate and petroleum ether (50:50) to afford yellowish syrup of the title compound.

Yield: 1.85 g, 81%.

¹H NMR (CDCl₃, 400 MHz): δ 7.18 (d, J=8.8 Hz, 1H), 6.58 (d, J=2.8 Hz, 1H), 6.48 (dd, J=2.8 & 6.0 Hz, 1H), 4.58 (t, J=5.0 Hz, 2H), 4.38 (t, J=5.0 Hz, 2H), 4.22 (s, 3H), 3.75 (s, 3H), 2.84 (s, 3H), 2.83 (t, J=7.6 Hz, 2H), 2.05-1.90 (m, 2H), 1.82-1.75 (m, 2H), 1.45 (s, 3H), 1.05-0.96 (m, 6H).

Mass (ES): m/z 492 (M⁺+1).

IR (cm⁻¹) (KBr): 2962, 1738, 1687, 1588, 1484.

### EXAMPLE 102

### R-(+)-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid

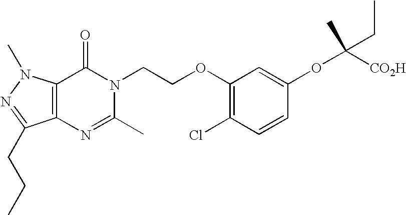

R-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid methyl ester (1.80 grams, 3.66 mmol), obtained in example 101, dissolved in a mixture of acetone and water (1:1, 25 mL), was added with sodium hydroxide (290 mg, 7.33 mmol) at 20 to 40° C. The reaction mixture was then refluxed for 1.5 hours. After cooling to 20 to 40° C., this mass was poured over ice-water, stirred, acidified with 6 N hydrochloric acid, and extracted with ethyl acetate (3×30 mL). The combined organic layer was washed with water, dried over anhydrous sodium sulphate and evaporated to get a off white gummy mass which was purified by column chromatography using 230-400 mesh silica-gel and mixture of ethyl acetate and petroleum ether (60:40). A further trituration of the gummy mass with ethyl acetate and petroleum ether (50:50) yielded a colorless solid of the title compound.

Yield: 1.50 g, 86%.

Melting Point: 98-100° C.

¹H NMR (CDCl₃, 400 MHz): δ 7.19 (d, J=8.4 Hz, 1H), 6.58 (d, J=2.8 Hz, 1H), 6.47 (dd, J=2.4 & 6.4 Hz, 1H), 4.53 (t, J=4.8 Hz, 2H), 4.31 (t, J=4.8 Hz, 2H), 4.20 (s, 3H), 2.84 (s, 3H), 2.83 (t, J=7.6 Hz, 2H), 2.10-1.90 (m, 2H), 1.80-1.75 (m, 2H), 1.46 (s, 3H), 1.03-0.97 (m, 6H).

Mass (CI): m/z 477 (M⁺+1).

IR (cm⁻¹) (KBr): 3500, 2960, 1721, 1689, 1579, 1491, 1465.

[α]²⁵_{D}+10.4 (c=0.25%, MeOH).

### EXAMPLE 103

### S-(−)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid methyl ester

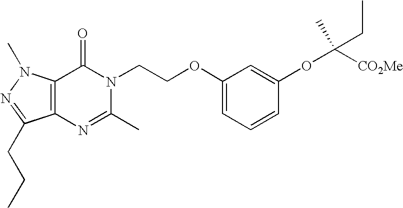

S-(−)-2-(3-Hydroxyphenoxy)-2-methyl butyric acid methyl ester (200 mg, 0.89 mmol), obtained in preparation 68, dissolved in toluene (10 ml), was added with anhydrous potassium carbonate (0.25 grams, 1.78 mmol), and refluxed for about 1.0 hour using Dean-Stark water separator to remove water content of the reaction mixture. After cooling to 20 to 40° C., 6-(2-chloroethyl)-1,5-dimethyl-3-propyl-1,6-dihydropyrazolo[4,3-d]pyrimidin-7-one (228 mg, 0.84 mmol) and tetra-n-butylammonium bromide (40 mg, 0.12 mmol) were subsequently added, and the reaction mixture was further refluxed for 20 hours. The reaction mixture was cooled to 20 to 40° C., poured over ice-water, stirred and extracted with ethyl acetate (3×10 mL). The combined organic layer was washed with water, dried over anhydrous sodium sulphate and evaporated to get a brown colored gummy mass which was purified by column chromatography using 230-400 mesh silica-gel and mixture of ethyl acetate and petroleum ether (50:50) to afford title compound.

Yield: 350 mg, 86%.

¹H NMR (CDCl₃, 400 MHz): δ 7.07 (t, J=7.6 Hz, 1H), 6.50 (dd, J=5.6 & 1.2 Hz, 1H), 6.39 (dd, J=2.4 & 1.6 Hz, 1H), 6.36 (dd, J=7.2 & 1.6 Hz, 1H), 4.46 (t, J=5.2 Hz, 2H), 4.26 (t, J=5.2 Hz, 2H), 4.21 (s, 3H), 3.74 (s, 3H), 2.83 (t, J=7.2 Hz, 2H), 2.74 (s, 3H), 2.04-1.90 (m, 2H), 1.85-1.75 (m, 2H), 1.48 (s, 3H), 1.05-0.96 (m, 6H).

Mass (CI): m/z 457 (M⁺+1).

IR (cm⁻¹) (Neat): 2961, 1735, 1690, 1577, 1487.

### EXAMPLE 104

### S-(−)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid

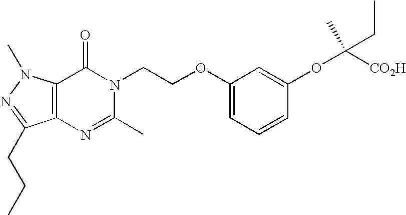

S-(−)-2-{3-[2-(1,5-Dimethyl-7-oxo-3-propyl-1,7-dihydro-pyrazolo[4,3-d]pyrimidin-6-yl)-ethoxy]-phenoxy}-2-methyl-butyric acid methyl ester (350 mg, 0.76 mmol), obtained in example 103, dissolved in a mixture of acetone and water (1:1, 10 mL), was added with sodium hydroxide (76 mg, 1.90 mmol) at 20 to 40° C. The reaction mixture was then refluxed for 1.5 hours. After cooling to 20 to 40° C., this mass was poured over ice-water, stirred, acidified with 6 N hydrochloric acid, and extracted with ethyl acetate (3×10 mL). The combined organic layer was washed with water, dried over anhydrous sodium sulphate and evaporated to get an off white gummy mass which was purified by column chromatography using 230-400 mesh silica-gel and mixture of ethyl acetate and petroleum ether (60:40). A further trituration of the gummy mass with ethyl acetate and petroleum ether (50:50) yielded a colorless title compound.

Yield: 300 mg, 88%.

Melting Point: 119-120° C.

¹H NMR (CDCl₃, 400 MHz): δ 7.11 (t, J=8.4 Hz, 1H), 6.61-6.50 (m, 2H), 6.48-6.47 (m, 1H), 4.46 (t, J=5.2 Hz, 2H), 4.27 (t, J=5.2 Hz, 2H), 4.21 (s, 3H), 2.85 (t, J=7.6 Hz, 2H), 2.73 (s, 3H), 2.10-1.90 (m, 2H), 1.80-1.77 (m, 2H), 1.48 (s, 3H), 1.03-0.95 (m, 6H).

Mass (CI): m/z 443 (M⁺+1).

IR (cm⁻¹) (KBr): 3442, 2965, 1688, 1601, 1492, 1469.

[α]²⁵_{D}−7.5 (c=1.0%, MeOH).

### EXAMPLE 105

### S-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid methyl ester

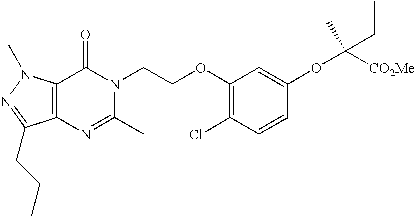

S-2-(4-Chloro-3-hydroxy-phenoxy)-2-methyl butyric acid methyl ester (1.15 grams, 4.44 mmol), obtained in preparation 74, dissolved in toluene (15 ml), was added with anhydrous potassium carbonate (1.23 grams, 8.88 mmol), and refluxed for about 1.0 hours using Dean-Stark water separator to remove water content of the reaction mixture. After cooling to 20 to 40° C., 6-(2-chloroethyl)-1,5-dimethyl-3-propyl-1,6-dihydropyrazolo[4,3-d]pyrimidin-7-one (1.13 grams, 4.21 mmol) and tetra-n-butylammonium bromide (230 mg, 0.71 mmol) were subsequently added, and the reaction mixture was further refluxed for 20 hours. The reaction mixture was cooled to 20 to 40° C., poured over ice-water, stirred and extracted with ethyl acetate (3×20 mL). The combined organic layer was washed with water, dried over anhydrous sodium sulphate and evaporated to get a brown colored gummy mass which was purified by column chromatography using 230-400 mesh silica-gel and mixture of ethyl acetate and petroleum ether (50:50) to afford a yellowish syrup of the titled compound.

Yield: 1.90 grams, 87%.

¹H NMR (CDCl₃, 400 MHz): δ 7.20 (d, J=8.8 Hz, 1H), 6.59 (d, J=2.8 Hz, 1H), 6.47 (dd, J=2.8 & 6.0 Hz, 1H), 4.55 (t, J=5.0 Hz, 2H), 4.39 (t, J=5.0 Hz, 2H), 4.21 (s, 3H), 3.76 (s, 3H), 2.85 (s, 3H), 2.84 (t, J=7.6 Hz, 2H), 2.06-1.90 (m, 2H), 1.81-1.75 (m, 2H), 1.44 (s, 3H), 1.05-0.95 (m, 6H).

Mass (CI): m/z 492 (M⁺+1).

IR (cm⁻¹) (KBr): 2961, 1738, 1688, 1587, 1484.

### EXAMPLE 106

### S-(−)-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid

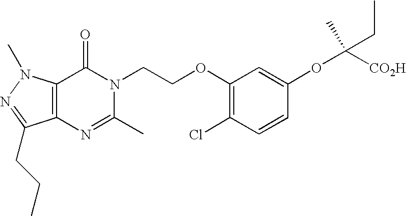

S-2-{4-Chloro-3-[2-(1,5-dimethyl-7-oxo-3-propyl-1,7-dihydro-pyrazolo[4,3-d]pyrimidin-6-yl)-ethoxy]-phenoxy}-2-methyl-butyric acid methyl ester (1.85 g, 3.76 mmol), obtained in example 105, dissolved in a mixture of acetone and water (1:1, 25 mL), was added with sodium hydroxide (376 mg, 9.41 mmol) at 20 to 40° C. The reaction mixture was then refluxed for 1.5 hours. After cooling to 20 to 40° C., this mass was poured over ice-water, stirred, acidified with 6 N hydrochloric acid, and extracted with ethyl acetate (3×30 mL). The combined organic layer was washed with water, dried over anhydrous sodiumsulphate and evaporated to get an off white gummy mass which was purified by column chromatography using 230-400 mesh silica-gel and mixture of ethyl acetate and petroleum ether (60:40). A further trituration of the gummy mass with ethyl acetate and petroleum ether (50:50) yielded a colorless solid of the title compound.

Yield: 1.55 grams, 86%.

Melting Point: 99-100° C.

¹H NMR (CDCl₃, 400 MHz): δ 7.18 (d, J=8.4 Hz, 1H), 6.59 (d, J=2.8 Hz, 1H), 6.48 (dd, J=2.4 & 6.4 Hz, 1H), 4.54 (t, J=4.8 Hz, 2H), 4.32 (t, J=4.8 Hz, 2H), 4.21 (s, 3H), 2.85 (s, 3H), 2.83 (t, J=7.6 Hz, 2H), 2.10-1.90 (m, 2H), 1.82-1.75 (m, 2H), 1.47 (s, 3H), 1.00-0.95 (m, 6H).

Mass (CI): m/z 477 (M⁺+1).

IR (cm⁻¹) (KBr): 3485, 2962, 1722, 1689, 1579, 1491, 1465.

[α]²⁵_{D}−12.2 (c=0.25%, MeOH)

We claim:

1. A derivative, which is a compound and/or a pharmaceutically acceptable salt of said compound, wherein said compound has the formula (I): 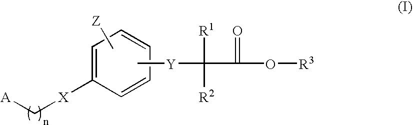
wherein
A has the structure 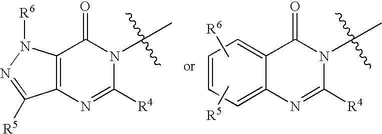
X is chosen from —CH₂—, —O—, —NH—, and —S—;
Y is chosen from —O—, —NH—, and —S—;
Z, which may be located in any position of substitution, is hydrogen or halogen;
R¹ and R², which may be the same or different, are independently chosen from hydrogen and C₁-C₈ alkyl, or R¹ and R² together form a carbocyclic ring having from 4 to 6 carbon atoms;
R³ is chosen from hydrogen and C₁-C₈ alkyl;
R⁴, R⁵, and R⁶, which may be the same or different, are independently chosen from hydrogen and C₁-C₈ alkyl; and
n is 1 to 6.

2. The derivative of claim 1, wherein A has the structure

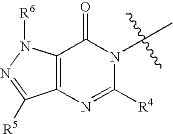

3. The derivative of claim 2, wherein Y is —O—.

4. The derivative of claim 3, wherein said compound has the formula (II): 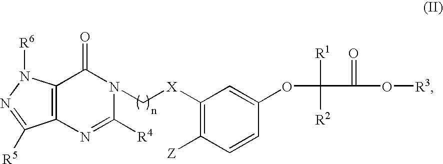
wherein
X is —O— or —CH₂—;
Z is hydrogen or chloro;
R¹ and R², which may be same or different, are independently chosen from methyl and ethyl;
R³ is chosen from hydrogen and C₁-C₅ alkyl;
R⁴, R⁵, and R⁶, which may be the same or different, are independently chosen from hydrogen and C₁-C₆ alkyl; and
n is 2, 3 or 4.

5. The derivative of claim 4, wherein X is —O—.

6. The derivative of claim 5, wherein R¹ is methyl, and R² is ethyl, R⁴, R⁵, and R⁶, which may be the same or different, are independently chosen from C₁-C₆ alkyl; and n is 2.

7. The derivative of claim 4, wherein X is —CH₂—.

8. The derivative of claim 7, wherein R¹ is methyl, R² is ethyl; R⁴, R⁵, and R⁶, which may be the same or different, are independently chosen from C₁-C₆ alkyl; and n is 2.

9. The derivative of claim 3, wherein said compound has the formula (III): 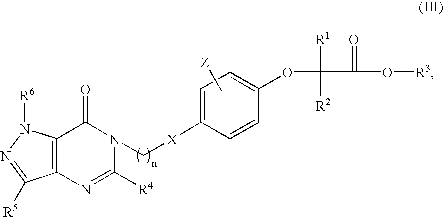
wherein
X is —O—, —NH— or —CH₂—;
Z is hydrogen or chloro;
R¹ and R², which may be same or different, are independently chosen from hydrogen, methyl and ethyl;
R³ is chosen from hydrogen and C₁-C₅ alkyl;
R⁴, R⁵, and R⁶, which may be the same or different, are independently chosen from hydrogen and C₁-C₆ alkyl; and
n is 2, 3 or 4.

10. The derivative of claim 9, wherein X is —CH₂—.

11. The derivative of claim 10, wherein R¹ is methyl, and R² is ethyl, R⁴, R⁵, and R⁶, which may be the same or different, are independently chosen from C₁-C₆alkyl; and n is 2.

12. The derivative of claim 2, wherein Y is —S— or —NH—.

13. The derivative of claim 12, wherein said compound has the formula (IV): 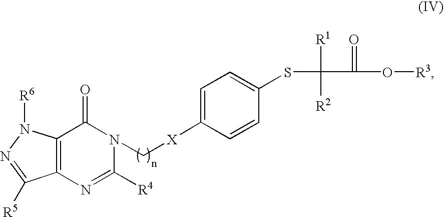
wherein
X is —O— or —NH—;
R¹ and R², which may be the same or different, are independently chosen from hydrogen and C₁-C₄ alkyl;
R³ is chosen from hydrogen and C₁-C₅ alkyl;
R⁴, R⁵, and R⁶, which may be the same or different, are independently chosen from C₁-C₆ alkyl; and
n is 2, 3 or 4.

14. The derivative of claim 13, wherein X is —O—.

15. The derivative of claim 14, wherein R¹, R², R⁴, and R⁶ are independently chosen from C₁-C₄ alkyl.

16. The derivative of claim 12, wherein said compound has the formula (V): 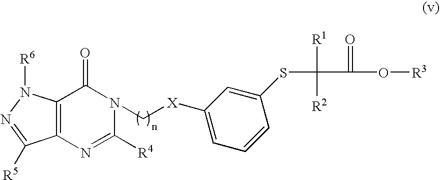
wherein
X is —O— or —NH—;
R¹ and R², which may be the same or different, are independently chosen from hydrogen and C₁-C₄ alkyl;
R³ is chosen from hydrogen and C₁-C₅ alkyl;
R⁴, R⁵, and R⁶, which may be the same or different, are independently chosen from C₁-C₆ alkyl; and
n is 2, 3 or 4.

17. The derivative of claim 16, wherein X is —O—.

18. The derivative of claim 2, wherein R⁵ is n-propyl.

19. The derivative of claim 2, wherein Z is hydrogen.

20. The derivative of claim 2, wherein R⁶ is methyl.

21. The derivative of claim 2, wherein R⁴ is C₁-C₃ alkyl.

22. The derivative of claim 2, wherein R⁴ is methyl.

23. The derivative of claim 2, wherein R³ is hydrogen.

24. The derivative of claim 1, wherein said compound has the structure

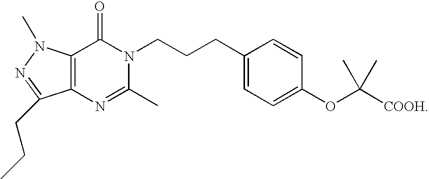

25. The derivative of claim 1, wherein said compound has the structure

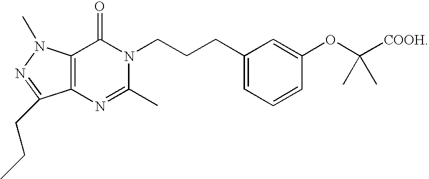

26. The derivative of claim 1, wherein said compound has the structure

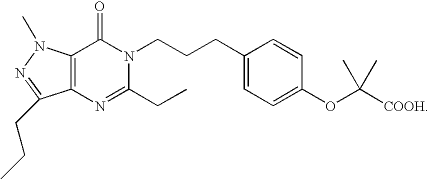

27. The derivative of claim 1, wherein said compound has the structure

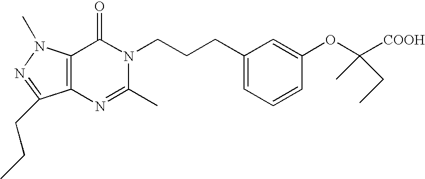

28. The derivative of claim 1, wherein said compound has the structure

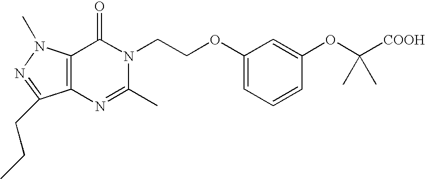

29. The derivative of claim 1, wherein said compound has the structure

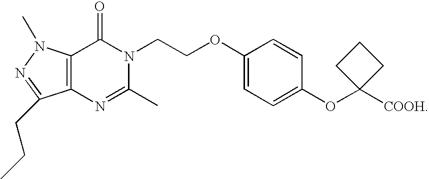

30. The derivative of claim 1, wherein said compound has the structure

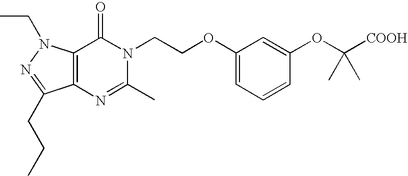

31. The derivative of claim 1, wherein said compound has the structure

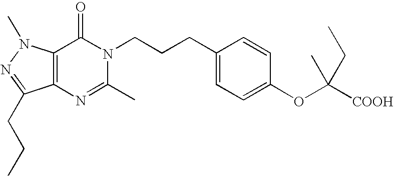

32. The derivative of claim 1, wherein said compound has the structure

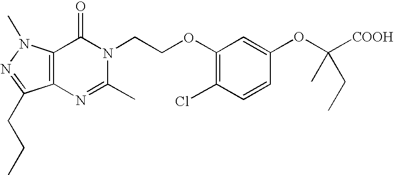

33. The derivative of claim 32, wherein said compound has the structure

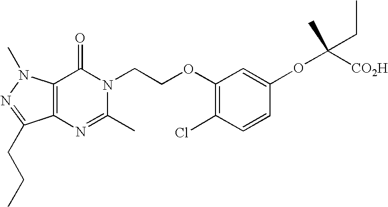

34. The derivative of claim 32, wherein said compound has the structure

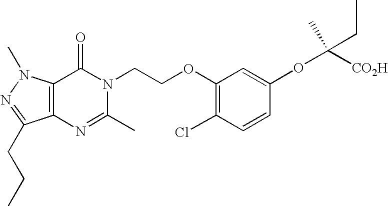

35. The derivative of claim 1, wherein said compound has the structure

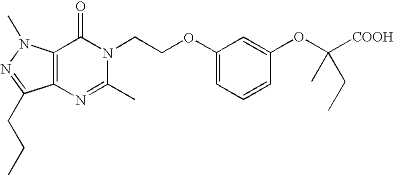

36. The derivative of claim 35, wherein said compound has the structure

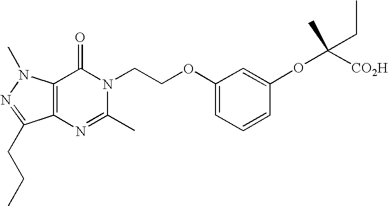

37. The derivative of claim 35, wherein said compound has the structure

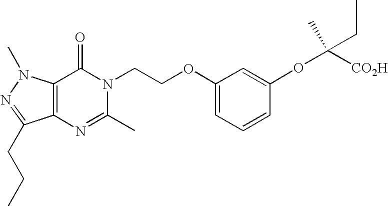

38. The derivative of claim 1, wherein said compound has the structure

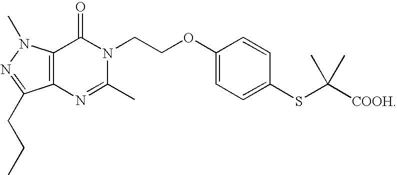

39. The derivative of claim 1, wherein said compound has the structure

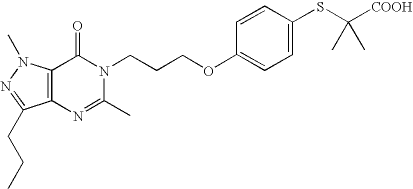

40. The derivative of claim 1, wherein said compound has the structure

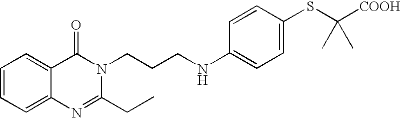

41. The derivative of claim 1, wherein said compound has the structure

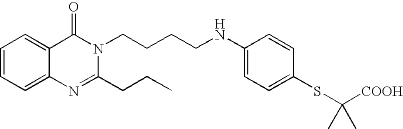

42. The derivative of claim 1, wherein said compound has the structure

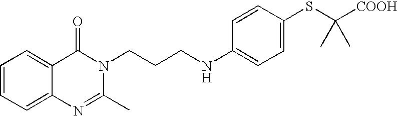

43. An ester prodrug of the derivative of claim 1 in accordance with the formula (I), in which R³ is not (C₁-C₈) alkyl.

44. The derivative of claim 1, wherein said compound is a stereoisomer.

45. The derivative of claim 44, wherein said compound has the structure 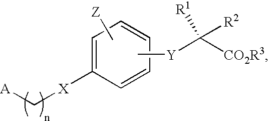
wherein R¹ and R² are different.

46. The derivative of claim 44, wherein said compound has the structure 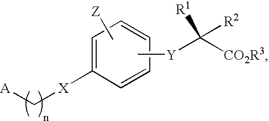
wherein R¹ and R² are different.

47. The derivative of claim 1, which is the pharmaceutically-acceptable salt of the compound in accordance with the formula (I).

48. The derivative of claim 1, which possesses PPARα activity of at least 1.5 at a 1 μM concentration and of at least 4 at a 10 μM concentration.

49. The derivative of claim 48, which possesses PPARα activity of at least 3.5 at a 1 μM concentration and of at least 6 at a 10 μM concentration.

50. The derivative of claim 48, which further possesses a PPARγ activity of at least 1.5 at a 1 μM concentration.

51. A method of producing a PPARα agonist activity in a mammal, said method comprising administering to said mammal an effective amount of the derivative of claim 1.

52. A method of producing a PPARα agonist activity in a mammal, said method comprising administering to said mammal an effective amount of the derivative of claim 48.

53. A method of producing a PPARα agonist activity and a PPARγ agonist activity in a mammal, said method comprising administering to said mammal an effective amount of the derivative of claim 50.

54. A pharmaceutical composition comprising the derivative of claim 1 and one or more pharmaceutically-acceptable excipients.
